(19) United States
(12) Patent Application Publication
(10) Pub. No.: US 2014/0220152 A1
(43) Pub. Date: Aug. 7, 2014

(54) Compositions and Methods for Detecting, Treating, or Preventing Reductive Stress

(71) Applicants: Ivor J. Benjamin, Salt Lake City, UT (US); Namakal S. Rajasekaran, Salt Lake City, UT (US); Thomas P. Kennedy, Charlotte, NC (US)

(72) Inventors: Ivor J. Benjamin, Salt Lake City, UT (US); Namakal S. Rajasekaran, Salt Lake City, UT (US); Thomas P. Kennedy, Charlotte, NC (US)

(73) Assignee: University of Utah Research Foundation, Salt Lake City, UT (US)

(21) Appl. No.: 14/025,750

(22) Filed: Sep. 12, 2013

Related U.S. Application Data
(63) Continuation of application No. 12/443,882, filed on Oct. 26, 2009, now abandoned.
(60) Provisional application No. 60/885,568, filed on Jan. 18, 2007.

(51) Int. Cl. A61K 31/325; A61K 31/4164; A61K 31/7008; A61K 31/5685; A61K 33/24; A61K 33/34; A61K 33/30; A61K 33/38; A61K 31/40; A61K 33/36

(52) U.S. Cl. CPC A61K 31/325; A61K 31/40; A61K 31/4164; A61K 31/7008; A61K 33/36; A61K 33/24; A61K 33/245; A61K 33/34; A61K 33/30; A61K 33/38; A61K 31/5685
USPC 424/618; 514/476; 514/423; 514/399; 514/62; 424/621; 424/646; 424/653; 424/637; 424/655; 424/641; 514/171

(57) ABSTRACT

Disclosed herein is a non-human animal model of protein aggregation cardiomyopathy. Also disclosed are compositions and methods of treating or preventing a condition in a subject caused or exacerbated by reductive stress. Also disclosed are compositions and methods of predicting, detecting, or monitoring reductive stress in a subject.

### CROSS-REFERENCE TO RELATED APPLICATIONS

This application is a continuation of U.S. patent application Ser. No. 12/443,882 filed Oct. 26, 2009, which is a national phase application of International Application No. PCT/US2008/051488 filed Jan. 18, 2008, which claims the benefit of U.S. Provisional Application No. 60/885,568 filed Jan. 18, 2007, each application of which is hereby incorporated herein by reference in its entirety.

### STATEMENT REGARDING FEDERALLY SPONSORED RESEARCH

This invention was made with government support under Grant 5RO1 HL63874 awarded by the National Heart, Lung, and Blood Institute (NHLBI). The government has certain rights in the invention.

### BACKGROUND

Heart failure encompasses both acquired (e.g., ischemia, myocarditis, valvular disease) and inheritable conditions (e.g., genetic cardiomyopathy) with disproportionate and increasing health and economic burdens for industrialized societies (Benjamin, I. J., et al. 2005; Morita, H., et al. 2005). Regardless of the etiology, mode of onset, and rate of progression, a common expression of this complex syndrome is the organism's inability to meet the peripheral metabolic demands. In general, current evidence-based therapeutic interventions for heart failure primarily target the end-stage manifestations (e.g., volume overload), without regard for the etiology and, often, with unpredictable consequences for the individual patient. If the goals of personalized medicine will soon be realized, then significant breakthroughs that improve early detection, guide targeted therapies and enhance disease monitoring are needed to combat heart failure in the genomic era (Bell, J. 2004; Seo, D., et al. 2006).

To date, eight mutations of the gene encoding the small heat shock protein, alpha B-crystallin (CryAB) have attributed to multisystem disorders of variable onset ranging from cataracts to respiratory failure to skeletal myopathy and cardiomyopathy (Vicart, P., et al. 1998; Goebel, H. H., et al. 2000; Wang, X., et al. 2001). Considerable heterogeneity in clinical characteristics among family members and the lack of diagnostic specificity on muscle biopsy, however, have raised questions about such classifications, especially pertaining to insights about the underlying cellular mechanisms (Dalakas, M. C., et al. 2000). On the basis of morphological, immunohistochemical and ultrastructural features in both humans and in mice, for example, the R120GCryAB mutation has multiple names including desmin-related myopathy (DRM), protein surplus myopathy, αB-crystallinopathy, myofibrillar disease with cardiomyopathy, and cardiac amyloidosis (Vicart, P., et al. 1998; Goebel, H. H., et al. 2000; Wang, X., et al. 2001; Sanbe, A., et al. 2004). As significant differences in the natural history exist among disease-causing CryAB mutations, need are genomic analyses to provide greater insights into molecular heterogeneity and biological subtypes for predicting outcomes.

Gene expression profiling has significantly improved the diagnostic classification of specific diseases (e.g., breast cancer, chronic myelogenous leukemia) by providing a ‘molecular signature’ and meaningful insights of the biological mechanisms underlying disease pathogenesis (Quackenbush, J. 2006). Much like the success seen for tumor classification and other improvements in cancer therapeutics (Bell, J. 2004; Quackenbush, J. 2006), and beyond the genetic tests for disease-causing mutations (Morita, H., et al. 2005), new genomic tools can provide novel approaches for molecular phenotyping of inheritable cardiomyopathy. Although transcriptional reprogramming of the human diseased hearts has been described (Seidman, J. G., et al. 2001), genetic models of single-gene disorders are robust platforms to combine precise phenotypic data to computational strategies that integrate cellular processes and biological networks.

αB-crystallin (CryAB), a small MW heat shock protein (Hsp) and molecular chaperone, is abundantly expressed in the heart and skeletal muscle and functions to prevent the aggregation of client proteins such as desmin, an intermediate filament cytoskeletal protein, thus maintaining muscle integrity and stress tolerance (Benjamin, I. J., et al. 1998; Taylor, R. P., et al. 2005). Protein aggregation skeletal myopathy and cardiomyopathy, which are caused by mutations in CryAB or desmin, are characterized by protein misfolding and large cytoplasmic aggregates (Goldfarb, L. G., et al. 1998; Vicart, P., et al. 1998; Dalakas, M. C., et al. 2000). Although in many instances only desmin and not CryAB is mutated, both CryAB and desmin accumulate ultrastructurally in dense granulomatous aggregates, hence the term desmin-related myopathy (DRM) (Goldfarb, L. G., et al. 1998; Vicart, P., et al. 1998; Dalakas, M. C., et al. 2000). Needed is an understanding of the pathogenesis of DRM in order to uncover new nodal pathways as potential targets of therapeutic interventions against heart failure (Benjamin, I.J.., et al. 2005).

### BRIEF SUMMARY

In accordance with the purpose of this invention, as embodied and broadly described herein, this invention relates to a non-human animal model of protein aggregation cardiomyopathy. The invention further relates to compositions and methods of treating or preventing a condition in a subject caused or exacerbated by reductive stress. The invention further relates to compositions and methods of predicting, detecting, or monitoring reductive stress in a subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A shows protein expression of total CryAB in transgenic mice. Western blot analysis was performed on either the soluble (supernatant) or insoluble (pellet) fractions isolated from hearts at 24 weeks old non-transgenic (NTg), human αB-crystallin (hCryAB Tg), hR120G Low and hR120G High expressors.

FIG. 1B shows densitomery performed on CryAB bands using supernatant of NTg animals as standard to compare the other groups. The fold changes expressed in arbitrary units in which representative groups consisted 3 or more animals (*P<0.001).

FIG. 2A shows gross morphology of hearts from age-matched NTg, hCryAB Tg, hR120G Low, and hR120G High at 6 months age. Hearts of hR120G High exhibit ventricular enlargement along with biatrial thrombosis consistent with heart failure.

FIG. 2B shows histological examination of myocardial sections. Hearts were perfusion-fixed and paraffin-embedded and stained with Toludine Blue (TB). Protein aggregates appear as white ‘patches’ in cardiomyocytes devoid of TB in hR120G High hearts. Immunohistochemical analysis of tissue sections stained with anti-CryAB shows large protein aggregates.

FIG. 2C shows immunogold localization of CryAB and desmin within the myocardium of 6 month-old hR120H mice. Boxes (×10,000) in the top show the area magnified in the bottom (×25,000). CryAB was found with dense granulomatous materials in the myocardium associated with myofibrillar aggregates (right panels). Desmin was similarly found over dense granules and protein aggregates (left panels).

FIG. 2D shows quantitative analysis of Northern dot blots of congestive heart failure markers at 3- and 6-months. Quantitation was carried out on Northern blot signals from Ntg (N=3) samples as 1.0 arbitrary unit (AU). The signals for atrial natriuretic factor (ANF) (panels (i) and (ii)), brain natriuretic factor (BNF) (panels (iii) and (iv)), and CryAB (panels (vii) and (viiii)) are all increased, while phospholamban (PLN) (panels (v) and (vi)) expression is decreased with the development of heart failure in hR120G myopathic hearts.

FIG. 2E shows Kaplan-Meier survival curve. The survival rates for non-transgenic (NTg), wild-type CryAB (hCryAB Tg, lines 3241 and 3244), and R120G hCryAB Tg (lines 7313 and 7302, designated hR120G Low and hR120G High, respectively) were analyzed over a period of 80 weeks. The majority of hR120G High mice developed congestive heart failure and died between 24 and 65 weeks. In contrast, the majority of hR120G Low mice (˜85%) were alive after 80 weeks. No differences in mortality were observed between hCryAB Tg and nontransgenic littermates.

FIG. 3A-FIG. 3E shows viability of left ventricular myocytes 1 hour after isolation (Mean±SEM, n=4 isolations for each group. Ntg: non-transgenic wild type; hR120G Low: mice with low level of expression of R120G mutation; hR120G High: mice with high level of R120G mutation expression. Isolated left ventricular myocytes were incubated in culture medium at 30° C. in a 5% CO₂ atmosphere for 1 hour. At least 100 myocytes were observed with phase contrast microscopy (Nikon TMS), and the % with a normal rod shape was taken as an index of viability (FIG. 3B and FIG. 3C). FIG. 3D shows myocardial external work and maximal rates of contraction before, during, and after exposure to 300 nM Dobutamine in the isolated perfused Langendorff heart. External work (RPP) is represented as the product of heart rate (HR) and left ventricular developed pressure (LVDP) (FIG. 3D), while maximal rate of contraction (+dP/dt) is the derivative of the measured LVDP (FIG. 3E). Values are mean±SE. NTg, non-transgenic control; hR120GCryAB Low. *(P<0.05) NTG vs. hR120G Low.

FIG. 4A-FIG. 4F show protein expression profiles of Hsps. Heart extracts of 24-week old NTg, hCryAB Tg, hR120G Low and hR120G High mice were analyzed by SDS-PAGE and supernatant or pellet fractions were immunoblotted with the respective antibodies (anti-Hsp25, 70, and 90) (FIG. 4A, FIG. 4C). Quantification of Hsp25, 70 and 90 expression was obtained from the Western blots of supernatant and pellet fractions, and the relative intensities of the densitometry values are represented as mean arbitrary units (FIG. 4B, FIG. 4D). The Hsp25 and 70 were higher (*P<0.10, **P<0.05) in the supernatants of R120G High expressors (FIG. 4C), whereas in the pellet fractions the Hsp25 expression (†P<0.001) was seen only in the R120G High group (FIG. 4D). No significant change in Hsp70 expression was seen and the Hsp90 is constitutively expressed in the pellets of all the groups.

FIG. 5A-FIG. 5D show markers of oxidative stress altered by R120G expression. Levels of lipid peroxidation produced were assessed as TBA-reacting substrances malondialdehyde (MDA, *P<0.05) in the respective groups at 6 months (FIG. 5A). Immunoblots of protein carbonylation (FIG. 5B), a biomarker for redox stress, were obtained from TCA supernatants of the DNPH-treated heart homogenates, which were separated by SDS-PAGE and then probed with rabbit anti-DNP antibody. Rate of protein carbonylation is relatively less in hR120G High hearts as they had elevated GSH levels (Table 6). Densitometry analysis of the DNPH blot (FIG. 5C) shows a 50% reduction of carbonylated proteins in the 6-month old hR120G High hearts, indicating an altered redox state in these mice.

FIG. 6A-FIG. 6F show assay of Glucose-6-phosphate dehydrogenase (G6PD) and gamma-glutamate cysteine ligase (γ-GCS) activity, protein and mRNA expression. G6PD enzyme activity (FIG. 6A) and protein expression (FIG. 6B) were increased in 6-month old hR120G High expressors. Glucose-6-phospate or glucose-6-phosphogluconate was added with NADP and activity was assessed spectrophotometrically. FIG. 6D shows densitometry analysis of the protein bands is expressed in arbitrary units, which show 12-fold increases of G6PD in the transgenic hearts with hR120GCryAB expression (*P<0.05). FIG. 6E & FIG. 6F show G6PD, mRNA and other antioxidative and stress response pathways (e.g., Hsp25) were induced by hR120G expression. Total RNA was harvested at the indicated time for Ntg, hCryAB Tg, and hR120G High, and mRNA transcripts were analyzed by Northern blot using radio-labelled probes against for G6PD (FIG. 6F, panel (i)) and Hsp25 (FIG. 6F, panel (ii)) and gamma-GCS (FIG. 6F, panel (iii)). Heart tissue γ-GCS levels were determined by Western blot and ELISA using anti-gamma-GCS/glutamate cysteine ligase-Ab and they were indistinguishable among all experimental groups (FIG. 6B, FIG. 6D).

FIG. 7A-FIG. 7E show enzyme activity and protein expression of glutathione reductase, catalase and glutathione peroxidase-1. Shown are enzymatic activities of glutathione reductase (GSH-R) and protein expression at 6 months. Glutathione reductase (GSH-R), which catalyses the recycling of GSSG to GSH, exhibits increased activity and expression in heart homogenates by hR120G expression. Densitometry (FIG. 7E) revealed about 1.5 fold increase in the GSH-R protein expression (panel (i)) by hR120G High compared with other groups (*P<0.05). Mutant hR120G High overexpression enhanced the activity of GPx-1 (panel (ii)) and catalase (panel (iii)) (*P≦0.05), two vital antioxidant enzymes involved in quenching hydrogen peroxides and lipid hydroperoxides in the hearts (FIG. 7B, FIG. 7C). Increased catalase activity correlated directly with its protein expression (**P<0.02), whereas the GPx-1 protein expression was unaltered (FIG. 7D, FIG. 7E (panel (ii))).

FIG. 8A-FIG. 8B show protein-protein interaction between G6PD and desmin with small Hsps (CryAB/Hsp25). Reciprocal co-immunoprecipitations (Co-IP) were performed with anti-desmin, anti-G6PD, CryAB and anti-Hsp25 antibodies followed by immunodetection with either anti-CryAB or anti-Hsp25 antibodies on heart hemogenule supernatant fractions Immunoprecipitated samples were probed with the following antibodies of interest; (desmin/CryAB (FIG. 8B(i)), desmin/Hsp25 (FIG. 8B(i)), G6PD/CryAB (FIG. 8B(iii)), CryAB/G6PD (FIG. 8B(iv)), G6PD/Hsp25 (FIG. 8B(ii)) and Hsp25/G6PD (FIG. 8B(v)). G6PD interacted with both CryAB and Hsp25 chaperone and such interactions were more pronounced in the hR120G H mice. Co-IP with the respective antibodies was also evident for potential interactions between the G6PD enzyme and the small sHsps. Densitometry analysis of immunoblots (as shown in FIG. 8A) revealed that the interactions were more prominent and significant in the hR120G High group (*P≦0.05).

FIG. 9A-FIG. 9E show G6PD deficiency reversed several biochemical and molecular features of hR120G High cardiomyopathy in vivo. Experimental groups of age-matched for transgenic mice for Ntg, hR120G High, hR120G/G6PD^{mut} and G6PD^{mut} were assessed for G6PD activity at 6 months. Protein abundance for G6PD, Hsp25, CryAB, and MnSOD were similar in hR120G High and hR12G/G6PD^{mut} hearts. In contrast, the development of cardiac hypertrophy, assessed by heart weight/body weight ratio, was completely prevented in hR12G/G6PD^{mut}. Decreased G6PDH activity and molecular signatures corresponded with increased resistance of hR12G/G6PD^{mut} to the pro-reducing effects of hR120G High expression cardiomyopathy.

FIG. 10 shows a schematic representation for the interactions leading to imbalances of redox state in the R120G mutant CryAB mediated cardiomyopathy. Mutant hR120G evoked the ‘classical’ heat shock response and upregulation of Hsp25, a redox-dependent chaperone that upholds GSH synthesis through interactions with G6PDH. Increased activity of G6PD generated more reducing equivalents in the form of NADPH, a substrate of glutathione reductase that catalyzes the conversion of oxidized GSSG to molecules of GSH. De novo synthesis was inhibited through feedback inhibition of γ-GCS by GSH. Evidence for early induction of both glutathione peroxidase (GPx) and catalase is consistent with increased reactive oxygen species in the ontogeny of cardiac hypertrophy and heart failure. The dysregulation of GSH biosynthesis sets up a vicious cycle for pro-reducing tilt towards reductive stress. The myopathic effects such as increased GSH and cardiac hypertrophy were abrogated in deficient G6PD^{mut} transgenic mice, demonstrating a direct causal mechanism and potential high quality therapeutic target for hR120G High cardiomyopathy.

FIG. 11 shows hierarchical cluster analysis of experimental arrays. Log intensity ratios of all spots on each array were used to group the experimental samples into clusters. Sample groups are color coded as indicated by the legend at left. The dendrogram above the diagram indicates the similarity between the individual arrays. Arrays joined by shorter distances are more similar than arrays joined by longer distances. NTG=nontransgenic controls, WT=hCryAB^{WT}, R120G=hR120GCryAB.

FIG. 12 shows major gene expression changes in transgenic hearts. All known genes identified with at least a two-fold change in expression (P<0.005) are shown. Green intensities denote decreased expression and red intensities denote increased expression according to the color bar (top right) normalized to the non-transgenic (NTG) control. Genes were identified as having significant expression changes at 3 months, 6 months, or both. Genes without significant change at either 3- or 6 months are not represented on the heatmap. Genes were assigned to arbitrarily selected functional groups, but could participate in multiple processes. NTG=non-transgenic controls, hCryAB^{WT}=human wild type CryAB transgene, hR120GCryAB=human R120G mutant CryAB transgene.

FIG. 13A-FIG. 13B show expression validation for specific genes by Northern blot analysis. Band intensities were measured in Northern blots (FIG. 13A) by densitometry and normalized to 18S rRNA levels as a loading control. For each gene, significant differences between conditions were determined by ANOVA and Fisher's PLSD post-hoc test (FIG. 13B, panels (i)-(vi)). *P<0.001 vs. NTG at corresponding time point. †P<0.002 vs. hCryAB^{WT} at corresponding time point. ‡P<0.002 vs. hR120GCryAB at 3 months. N=3 RNA samples per group.

FIG. 14 shows summary of pairwise comparisons at 3 and 6 months. In each Venn diagram, circles represent the individual pairwise comparisons: NTG vs. hCryAB^{WT} (red circle), hCryAB^{WT} vs. hR120GCryAB, and NTG vs. hR120GCryAB. Numbers in parentheses after each comparison represent the total number of sequences with significant change in expression for that comparison. Numbers inside each compartment represents the number of sequences unique for that effect or, if intersecting with another circle, the number of sequences identified for the intersecting set. Note the small number of identified sequences attributable to the hCryAB^{WT} compared with the hR120GCryAB trans gene.

FIG. 15A-FIG. 15B show G6PD expression by Northern blot analysis. Northern blot band intensities were assessed by densitometry and normalized to 18S rRNA levels. Significant differences between conditions were determined by ANOVA and Fisher's PLSD post-hoc test. FIG. 15A shows representative Northern blot and corresponding 18S rRNA from ethidium bromide stained gel. FIG. 15B shows analysis of densitometry data presented as mean G6PD density/18S rRNA density ±SEM of three replicates. NTG=nontransgenic controls, WT=human wild type CryAB transgene (hCryAB^{WT}), R120G=human R120G mutant CryAB transgene (hR120GCryAB). *P<0.0005 vs. all other groups. †P=0.005 vs. hCryAB WT at 6 months.

FIG. 16A-FIG. 16B show mutant hR120GCryAB induces the HSP stress response pathway. FIG. 16A shows by Northern blots that Hsp25 transcripts are significantly increased in hR120GCryAB High Tg hearts at 3 and 6 month old animals (*p<0.05 versus 3 month NTg). FIG. 16B shows quantification of Northern blot data. All results represent mean±SD of 3-6 animals/group.

FIG. 17A-FIG. 17B show enzyme activity and expression of glutathione peroxidase-1 (GPx-1) and catalase at 6 months. FIG. 17A shows mutant hR120GCryAB High Tg overexpression enhances the activities of GPx-1 and FIG. 17B shows mutant hR120GCryAB High Tg enhances overexpression of catalase (*p˜0.05) in 6 month old hearts.

FIG. 17C shows Northern blot analysis using radio-labeled cDNA probes against GPx-3 and catalase (Cat). Total RNA was harvested from NTg, hCryAB Tg and hR120GCryAB High Tg at either 3 or 6 months.

FIG. 17D and FIG. 17E show densitometry analysis of Northern blots of FIG. 2E expressed in arbitrary units shows 2-3 fold increases for GPx-3 and 5-fold increase for catalase, in both 3 and 6 month old hR120GCryAB High Tg hearts (*p<0.05 versus 3 month NTg). Each lane represents an individual animal (3 animals/group). All results represent mean±SD of 3-6 animals/group.

FIG. 18A-FIG. 18D show hR120GCryAB overexpression enhances antioxidative enzymatic and GSH recycling pathways. FIG. 18A shows glutathione reductase, which catalyzes the recycling of GSSG to GSH, exhibits increased activity and expression in heart homogenates with hR120GCryAB High Tg expression at 6 months (*p<0.05 versus NTg). FIG. 18B shows representative Western blot analysis of G6PD, GSH-R and g-GCS protein expression in 6 month old hR120GCryAB High Tg animals. FIG. 18C and FIG. 18D show densitometry analysis of the protein bands expressed in arbitrary units shows 4 fold increase of G6PD (n=6) and 40% increase of GSH-R in the transgenic hearts with hR120GCryAB High expression compared to NTg, respectively (*p<0.05). All results represent mean±SD of >6 animals/group.

FIG. 19A-FIG. 19B shows hR120GCryAB overexpression promotes colocalization and interactions between G6PD and Hsp25 in protein aggregates. FIG. 19A shows representative Westerns of supernatant fractions from heart homogenate after coimmunoprecipitation were performed and probed with antiG6PD, anti-CryAB and anti-Hsp25 antibodies. A vertical bar (j) indicates cropped lanes made in the original gel image to remove irrelevant spaces. FIG. 19B shows densitometry analysis of immunoblots indicates significant interactions among CryAB, Hsp25 and G6PD in the hR120GCryAB High Tg group. G6PD/CryAB (panels a and b); CryAB/G6PD (panels a and b); G6PD/Hsp25 (panels c and d); and Hsp25/G6PD (panels c and d). (*p<0.05, **p<0.01 compared with NTg control).

FIG. 20 shows schematic diagram illustrates the different etiologies and multiple compensatory and adaptive pathways implicated in the clinical syndrome of heart failure.

FIG. 21A-FIG. 21B show ventricular remodeling after infarction ( [symbol] FIG. 21A) and in diastolic heart failure (FIG. 21B). (Adapted from Jessup et al, N Engl J Med 348:2007-2018).

FIG. 22 shows at the cellular and molecular levels, crosstalk among pathways related to oxidative stress and calcium dysregulation, for example, may contribute to secondary apoptosis and necrosis. In spite of considerable insights about mechanisms, current therapies focus on reversing neurohumoral imbalances but rarely on underlying mechanisms.

FIG. 23 shows new diagnostic approaches, based on information that integrates genes and molecular pathways at the onset, progression and end stages are needed to improve heart failure classification. ‘Biosignatures’ for heart failure—developed from microarray analysis technologies, proteomics and genomic technologies—are disclosed here to integrate the biological processes and molecular mechanisms for rationale drug design and treatment in the post genomic era of personalized medicine.

FIG. 24 shows human gene mutations can cause cardiac hypertrophy, dilation, or both. In addition to these two patterns of remodeling, particular gene defects produce hypertrophic remodeling with glycogen accumulation or dilated remodeling with fibrofatty degeneration of the myocardium. Sarcomere proteins denote 3-myosin heavy chain, cardiac troponin T, cardiac troponin I,a-tropomyosin, cardiac actin, and titin. Metabolic/storage proteins denote AMPactivated protein kinase y subunit, LAMP2, lysosomal acid a 1,4-glucosidase, and lysosomal hydrolase agalactosidase A. Z-disc proteins denote MLP and telethonin. Dystrophin-complex proteins denote 6-sarcoglycan, 3-sarcoglycan, and dystrophin. Ca²⁺ cycling proteins denote PLN and RyR2. Desmosome proteins denote plakoglobin, desmoplakin, and plakophilin-2. (Adapted from Morita et al. J. Clin. Invest. 115: 518-526, 2005).

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a polypeptide is disclosed and discussed and a number of modifications that can be made to a number of molecules including the polypeptide are discussed, each and every combination and permutation of polypeptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. COMPOSITIONS

Among several disease-causing mutations identified for CryAB to date, the Arg120→Gly mutation causes an autosomal dominant, multisystem disorder characterized with variable onset of signs and symptoms including cardiomyopathy (Vicart, P., et al. 1998; Fardeau, M., et al. 1978). Earlier biochemical studies have described several consequences of hR120GCryAB on the integrity of protein structure (Kumar, L. V., et al. 1999), iin vitro chaperone-like activity (Bova, M. P., et al. 1999), propensity for aggregation with intermediate filaments and increased instability towards heat-induced protein denaturation (Perng, M. D., et al. 1999). In addition, misfolded proteins such as R120GCryAB are important stress signals for triggering adaptive mechanisms such as heat shock protein gene expression (Christians, E. S., et al. 2002). Because protein misfolding increases aberrancy and exposes the hydrophobic surfaces, many Hsp chaperones are recruited to repair damaged proteins, accelerate protein degradation and/or mitigate potential catastrophic events (Christians, E. S., et al. 2002; Xiao, X., et al. 1999). In particular, Hsp25 overexpression increases GSH content and confers oxidative resistance in L929 cells (Mehlen, P., et al. 1996; Baek, S. H., et al. 2000), whereas Hsp25 down-regulation linked to GSH depletion increases oxidative stress (Christians, E. S., et al. 2002).

As disclosed herein, interactions between misfolded protein expression and the glutathione-dependent redox state play a key role in the pathogenesis of hR120GCryAB cardiomyopathy. Disclosed herein are profound increases in reduced GSH concentrations and ratio of GSH/GSSG, due at least partially to increased conversion from oxidized GSSG to reduced GSH since the enzymatic activities of glucose-6-phosphate dehydrogenase (G6PD) (Baek, S. H., et al. 2000; Preville, X., et al. 1999), glutathione reductase, glutathione peroxidase, and catalase were significantly increased by dose-dependent hR120G expression. Because the biochemical and molecular consequences were reversed in double transgenic G6PD-deficient bred into hR120GCryAB cardiomyopathic (hR120G/G6PD^{mut}) mice, such evidence for the first time supports a causative link mechanism for ‘reductive stress’ in the pathogenesis of hR120G-induced cardiomyopathy.

Based on this discovery, disclosed herein are compositions, including animal models of protein aggregation cardiomyopathy, and methods, including methods of treating conditions caused by reductive stress generally or G6PD specifically.

### 1. Animal Models of Human Diseases

To further investigate the pathologic mechanisms of protein aggregation cardiomyopathy at the molecular level, transgenic mouse models recapitulating defined aspects of the human disease represent valuable tools for exploring disease pathogenesis. Wang and coworkers, for example, have exploited transgenic lines to implicate cardiac-specific expression of mouse R120G (mR120G) CryAB in myofibrillar impairment and cardiac hypertrophy mimicking DRM (Wang, X., et al. 2001). Disclosed herein transgenic mice harboring human R120GCryAB (hR120GCryAB) that fully recapitulate the morphological, functional, and molecular features of human CryAB cardiomyopathy.

Provided herein are non-human transgenic animals wherein nucleated cells of the animal comprise a nucleic acid encoding a human αB-crystallin (CryAB) protein operably linked to an expression control sequence, wherein the protein comprises a mutation at residue 120, wherein the non-human mammal exhibits one or more symptoms of protein aggregation cardiomyopathy. In some aspects, the expression control sequence is not a naturally occurring CryAB promoter and is therefore not operably linked to a nucleic acid encoding CryAB in nature.

Disclosed herein is a method of identifying molecules that play roles in the development of protein aggregation cardiomyopathy. Also disclosed is a method of elucidate the biological mechanism of the disease. Also disclosed is a method of testing therapeutic approaches for protein aggregation cardiomyopathy. In some aspects, the animal model is an insect, such as Drosophila. There are several reasons to choose Drosophila as a model system. First, the well-developed genetics of Drosophila and its short life cycle make it possible to carry out genetic screens that would be much more tedious, difficult and expensive in the mouse. Second, a number of protein-aggregation diseases associated with neuro-degeneration have been successfully modeled in Drosophila, including characterization of the effects of modifying genes. Third, Drosophila is unique among invertebrate models in having pumping hearts. Fourth, the Drosophila genome carries genes that are closely related to the human αB-crystallin, and there is evidence to support that they are performing similar functions.

To reproduce the disease phenotype in Drosophila disclosed is a two-pronged approach. First, the disease allele can be expressed with the strongest and most widespread phenotype (R120G) in Drosophila under control of the Gal4-UAS system. This system provides for the conditional and regulated expression of transgenes in virtually any tissue of the fly. Expression can be focused on in the compound eye, because the eye has a highly stereotypical pattern that is very sensitive for detecting disruptions of cellular function during development, and because the eye is dispensable for life. This system has proven utility for detecting interactions via genetic screens. The mutant protein can also be expressed in flight muscles, and in heart muscles to more precisely mimic the myopathies. The affected cells can be examined for the presence of dense protein aggregates to validate this aspect of the model. The protein can also be expressed in whole flies, which can then be tested for altered glutathione levels.

In case no phenotype is readily observed, and expression of the R120G protein is verified, several approaches can be used to generate a visible and genetically useful phenotype. In some circumstances Hsp70 cooperates with Hsp27 (Lee and Vierling 2000). ectopically-expressed R120G can be combined with Hsp70 gene deletions or duplications to test whether decreasing or increasing the dose of a partner can enhance the mutant phenotype. After expression in the eye, eye cells of aged adults can be examined for the presence of dense protein aggregates. If present, their appearance can be accelerated by combining R120G expression with G6PD overexpression.

Also disclosed is a method to reproduce DRM in Drosophila using gene targeting methods (Rong et al. 2002) to precisely engineer the R120G mutation into the fly homologs of the αB-crystallin gene. There are at least two genes that are closely and almost equally related to CryAB: Hsp27 and l(2)efl. Overexpression of Drosophila Hsp27 can cause increased glutathione levels, and mutation of the conserved arginine residue in the α-crystallin domains of four small MW Hsps (α-crystallin of αA-, αB-crystallin, HspB8 and hamster Hsp27) causes protein aggregates (Chavez Zobel, 2005). Additional mutations can be engineered in the remaining homologs, and this can be accomplished with current technology. the mutant flies can be examined for dominant and recessive effects, particularly with respect to viability, lifespan, and fertility. If single mutants have no phenotype, double mutants can be examined as well. Cells can also be examined for the presence of dense protein aggregates.

i. Animals

By a “transgene” is meant a nucleic acid sequence that is inserted by artifice into a cell and becomes a part of the genome of that cell and its progeny. Such a transgene may be (but is not necessarily) partly or entirely heterologous (e.g., derived from a different species) to the cell. The term “transgene” broadly refers to any nucleic acid that is introduced into an animal's genome, including but not limited to genes or DNA having sequences which are perhaps not normally present in the genome, genes which are present, but not normally transcribed and translated (“expressed”) in a given genome, or any other gene or DNA which one desires to introduce into the genome. This may include genes which may normally be present in the nontransgenic genome but which one desires to have altered in expression, or which one desires to introduce in an altered or variant form or in a different chromosomal location. A transgene can include one or more transcriptional regulatory sequences and any other nucleic acid, such as introns, that may be useful or necessary for optimal expression of a selected nucleic acid. A transgene can be as few as a couple of nucleotides long, but is preferably at least about 50, 100, 150, 200, 250, 300, 350, 400, or 500 nucleotides long or even longer and can be, e.g., an entire genome. A transgene can be coding or non-coding sequences, or a combination thereof. A transgene usually comprises a regulatory element that is capable of driving the expression of one or more transgenes under appropriate conditions. By “transgenic animal” is meant an animal comprising a transgene as described above. Transgenic animals are made by techniques that are well known in the art. The disclosed nucleic acids, in whole or in part, in any combination, can be transgenes as disclosed herein.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein.

The disclosed transgenic animals can be any non-human animal, including an invertebrate (e.g., insect) or vertebrate, in which one or more cells contain heterologous nucleic acid introduced by way of human intervention, such as by transgenic techniques well known in the art. Thus, the non-human animal can be a fly (e.g., drosophila). Moreover, the non-human animal can be a non-human mammal (e.g., mouse, rat, rabbit, squirrel, hamster, rabbits, guinea pigs, pigs, micro-pigs, prairie dogs, baboons, squirrel monkeys and chimpanzees, etc), bird or an amphibian. For example, the animal can be selected from the group consisting of avian, bovine, canine, caprine, equine, feline, leporine, murine, ovine, porcine, non-human primate. Thus, the animal can be a mouse, a rabbit, or a rat.

Generally, the nucleic acid is introduced into the cell, directly or indirectly, by introduction into a precursor of the cell, such as by microinjection or by infection with a recombinant virus. The disclosed transgenic animals can also include the progeny of animals which had been directly manipulated or which were the original animal to receive one or more of the disclosed nucleic acids. This molecule may be integrated within a chromosome, or it may be extrachromosomally replicating DNA. For techniques related to the production of transgenic animals, see, inter alia, Hogan et al (1986) Manipulating the Mouse Embryo—A Laboratory Manual Cold Spring Harbor Laboratory, Cold Spring Harbor, N.Y., 1986).

Animals suitable for transgenic experiments can be obtained from standard commercial sources such as Charles River (Wilmington, Mass.), Taconic (Germantown, N.Y.), and Harlan Sprague Dawley (Indianapolis, Ind.). For example, if the transgenic animal is a mouse, many mouse strains are suitable, but C57BL/6 female mice can be used for embryo retrieval and transfer. C57BL/6 males can be used for mating and vasectomized C57BL/6 studs can be used to stimulate pseudopregnancy. Vasectomized mice and rats can be obtained from the supplier. Transgenic animals can be made by any known procedure, including microinjection methods, and embryonic stem cells methods. The procedures for manipulation of the rodent embryo and for microinjection of DNA are described in detail in Hogan et al., Manipulating the Mouse Embryo (Cold Spring Harbor Laboratory, Cold Spring Harbor, N.Y., 1986), the teachings of which are generally known and are incorporated herein.

Transgenic animals can be identified by analyzing their DNA. For this purpose, for example, when the transgenic animal is an animal with a tail, such as rodent, tail samples (1 to 2 cm) can be removed from three week old animals. DNA from these or other samples can then be prepared and analyzed, for example, by Southern blot, PCR, or slot blot to detect transgenic founder (F (0)) animals and their progeny (F (1) and F (2)). Thus, also provided are transgenic non-human animals that are progeny of crosses between a transgenic animal of the invention and a second animal. Transgenic animals can be bred with other transgenic animals, where the two transgenic animals were generated using different transgenes, to test the effect of one gene product on another gene product or to test the combined effects of two gene products.

ii. Phenotype

As disclosed herein, disclosed non-human mammal comprising a nucleic acid encoding a human αB-crystallin (CryAB) protein operably linked to an expression control sequence, wherein the protein comprises a mutation at residue 120 exhibits protein aggregation cardiomyopathy. The phenotype of the disclosed non-human mammal wherein the mammal is a mouse is provided in Example 1.

iii. CryAB Transgene

The mutant CryAB protein of the disclosed non-human mammal can comprise a substitution of the arginine at residue 120 with an amino acid residue not arginine. For example, the substitution can be of the arginine at residue 120 of the reference sequence SEQ ID NO:1. However, this residue can also be identified in modified and/or truncated forms of CryAB wherein the amino acid is no longer at residue 120 based on its relative position in SEQ ID NO:1. For example, the CryAB protein can comprise a substitution of an arginine residue having substantially similar structural positioning as residue 120 of SEQ ID NO:1.

In some aspects, the substitution is non-conservative. For example, the substituted amino acid can be glycine.

The mutant CryAB protein can be a mutant form of any known or newly discovered mammalian CryAB protein wherein the functional equivalent of residue 120 of SEQ ID NO:1 can be identified. For example, the CryAB protein can comprise the amino acid sequence SEQ ID NO:3 or a fragment thereof of at least 100, 110, 120, 130, 140, 150, 160, or 170 amino acids.

The mutant CryAB protein can comprise an amino acid sequence having at least about 70%, at least about 75%, at least about 80%, at least about 85%, at least about 90%, at least about 91%, at least about 92%, at least about 93%, at least about 93%, at least about 94%, at least about 95%, at least about 96%, at least about 97%, at least about 98%, at least about 99%, or at least about 100% identity to SEQ ID NO:3, or a fragment thereof of at least 100, 110, 120, 130, 140, 150, 160, or 170 amino acids.

The nucleic acid encoding the mutant CryAB protein can comprise the nucleic acid sequence SEQ ID NO:4, 5, 6, or 7, or a fragment thereof of at least 100, 150, 200, 250, 300, 350, 400, 450, 500, 550, 600, or 650 nucleic acids. The nucleic acid encoding the mutant CryAB protein can comprise a nucleic acid sequence having at least about 70%, at least about 75%, at least about 80%, at least about 85%, at least about 90%, at least about 91%, at least about 92%, at least about 93%, at least about 93%, at least about 94%, at least about 95%, at least about 96%, at least about 97%, at least about 98%, at least about 99%, or at least about 100% identity to SEQ ID NO:4, 5, 6, or 7 or a fragment thereof of at least 100, 150, 200, 250, 300, 350, 400, 450, 500, 550, 600, or 650 nucleic acids.

The nucleic acid encoding the CryAB protein can hybridize under stringent conditions to a nucleic acid consisting of SEQ ID NO:4, 5, 6, or 7 or the complement of SEQ ID NO:4, 5, 6, or 7, or a fragment thereof of at least 100, 150, 200, 250, 300, 350, 400, 450, 500, 550, 600, or 650 nucleic acids.

iv. Expression Control Sequence

Nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

The nucleic acid encoding the expression control sequence can be heterologous to the animal. The expression control sequence can comprise a constitutive promoter. The expression control sequence can comprise a cell-specific promoter. For example, the cell-specific promoter can be muscle creatine kinase (MCK) promoter (Fabre et al. J Gene Med. 2006 8(5):636-45), desmin promoter (Raats et al. Eur J Cell Biol. 1996 71(3):221-36), or myoglobin promoter. The expression control sequence can comprise a cardiac-specific promoter, such as the ventricle-specific cardiac myosin light chain-2v promoter (MLC-2v). The expression control sequence can comprise a human cytomegalovirus (HCMV) immediate-early (IE) enhancer. The expression control sequence can comprise a chicken β-actin promoter with first intron (Niwa H et al, Gene (Amst). 1991; 108: 193-200).

The expression control sequence can comprise an inducible promoter. Alternatively, the nucleated cells of the provided animal can further comprise a transgene encoding a transactivator protein, wherein the transactivator protein conditionally induces expression of the transgene. For example, inducible expression by the transactivator protein can be conditioned on the presence of tetracycline or derivative thereof. Likewise, inducible expression by the transactivator protein can be conditioned on the absence of tetracycline or derivative thereof. Numerous other control sequences and systems are known and can be used with the disclosed transgenes and transgeneic animals.

a. Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. The transcription unit can also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b. Markers

The viral vectors can include nucleic acid sequence encoding a marker product. This marker product is used to determine if the gene has been delivered to the cell and once delivered is being expressed. Preferred marker genes are the E. coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR− cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 2. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 3. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 4. Nucleic Acids

There are a variety of molecules disclosed herein that are nucleic acid based, including for example the nucleic acids that encode, for example SEQ ID NOs:4, 5, 6, or 7, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

i. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

ii. Sequences

There are a variety of sequences related to the protein molecules involved in the signaling pathways disclosed herein, for example SEQ ID NO:1 and 3, which are encoded by nucleic acids or are nucleic acids. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

iii. Primers and Probes

Disclosed are compositions including primers and probes, which are capable of interacting with the disclosed nucleic acids, such as the SEQ ID NOs:4, 5, 6, or 7 as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### 5. Peptides

i. Protein Variants

As discussed herein there are numerous variants of the disclosed proteins that are known and herein contemplated. In addition, to the known functional variants there are derivatives of the proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking iin vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Table 1 and are referred to as conservative substitutions.

TABLE 1
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g., Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

It is understood that if a specific amino acid residue is identified as critical for function or relevant in disease, then the herein disclosed sequence identity is based on the non-critical amino acid residues. Thus, one of skill in the art can identify non-functional variants that fall within the disclosed sequence identity based on known, identified, or predicted critical amino acid residues.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:3 is set forth in SEQ ID NO:4. Another nucleic acid sequence that encodes the same protein sequence set forth in SEQ ID NO:3 is set forth in SEQ ID NOs:5, 6 and 7.

It is understood that there are numerous amino acid and peptide analogs which can be incorporated into the disclosed compositions. For example, there are numerous D amino acids or amino acids which have a different functional substituent then the amino acids shown in Table 1. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### B. METHODS

### 1. Method of Treating Heart Failure/Cardiomyopathy

Provided herein is a method of treating or preventing heart failure in a subject, comprising diagnosing a subject as having or at risk of developing heart failure and administering to the subject a composition comprising an inhibitor of glucose-6-phosphate dehydrogenase (G6PD).

i. Heart Failure

Congestive heart failure (CHF), also called congestive cardiac failure (CCF) or just heart failure, is a condition that can result from any structural or functional cardiac disorder that impairs the ability of the heart to fill with or pump a sufficient amount of blood throughout the body. Thus, the disclosed method can be used to treat any form of heart failure.

Because not all patients have volume overload at the time of initial or subsequent evaluation, the term “heart failure” is preferred over the older term “congestive heart failure”. Causes and contributing factors to congestive heart failure include the following (with specific reference to left (L) or right (R) sides): Genetic family history of CHF, Ischemic heart disease/Myocardial infarction (coronary artery disease), Infection, Alcohol ingestion, Heartworms, Anemia, Thyrotoxicosis (hyperthyroidism), Arrhythmia, Hypertension (L), Coarctation of the aorta (L), Aortic stenosis/regurgitation (L), Mitral regurgitation (L), Pulmonary stenosis/Pulmonary hypertension/Pulmonary embolism all leading to cor pulmonale (R), and Mitral valve disease (L).

There are many different ways to categorize heart failure, including: the side of the heart involved, (left heart failure versus right heart failure), whether the abnormality is due to contraction or relaxation of the heart (systolic heart failure vs. diastolic heart failure), and whether the abnormality is due to low cardiac output or low systemic vascular resistance (low-output heart failure vs. high-output heart failure).

a. Summary

Heart failure is a common clinical problem characterized by the inability of the heart to function as mechanical pump for maintaining the organism's peripheral metabolic demands. Regardless of the etiology, mode of onset, or rate of progression, current therapies and interventions for heart failure primarily tackle the relief of symptoms (e.g., shortness of breath) and signs (e.g., leg edema) upon clinical presentation. While remarkably effective when tested in large populations with different etiologies, such interventions do not correct the underlying conditions or predict the responses for an individual patient. As discussed further below, the existing clinical model used for generating evidence-based medicine and practice guidelines will become obsolete in the face of overwhelming costs associated with clinical trials, incremental benefits of new therapies, and the burgeoning clinical demand. If the goals of personalized medicine will soon be realized, then significant breakthroughs that improve early detection, guide targeted therapies and enhance disease monitoring are needed to combat heart failure in the genomic era (Bell, 2004; Park et al., 2006). Disclosed is the classification schemes, underlying pathophysiologic mechanisms and the potential challenges and opportunities for genomics to drive the emerging transformative discipline of cardiovascular biomedicine.

b. Definitions

Heart failure has been conveniently subdivided according to abnormalities in the cardiac cycle: namely, systolic heart failure (SHF) and diastolic heart failure (DHF). Systolic heart failure (SHF) is associated with decreased cardiac output and ventricular contractility, termed systolic dysfunction, and is attributed to a loss of ventricular muscle cells. Dilated cardiomyopathy is characterized by impaired systolic function and myocardial remodeling and enlargement of one or both ventricles. Idiopathic dilated cardiomyopathy (IDCM) refers to primary myocardial disease in the absence of coronary, valvular or systemic disease. The ventricular remodeling of diastolic heart failure, however, is characterized by normal chamber size without impaired ventricular filling from abnormal myocardial stiffness during the relaxation phase. More recently, the clinical syndrome of heart failure with preserved ejection fraction (HFPEF)—left ventricular ejection fraction >50 percent—has been recognized in several cross-sectional studies (Bhatia et al., 2006; Owan et al., 2006).

c. Predisposition (Genetic and Non-Genetic)

In western societies, ischemic heart disease (˜60 percent) and hypertension are the most common causes of ventricular systolic dysfunction but there is now irrefutable evidence for genetic defects whose onset and progression occur in adulthood (e.g., familial cardiomyopathy) (Benjamin and Schneider, 2005; Morita et al., 2005). Beginning in the late 50s, distinct alterations in the size and geometry of the left ventricle, termed ‘ventricular remodeling,’ were being recognized but the ensuing debate for over three decades was primarily focused on morphological classifications. Hypertrophic cardiomyopathy (HCM) is characterized by predominant and marked thickening of the left circumferential ventricular wall (i.e., hypertrophy), small LV cavity size and hypercontractility. Such patients including young athletes were prone to sudden cardiac death attributed pathophysiologically to subaortic stenosis and cavity obliteration triggering inadequate cardiac output and lethal arrhythmias. In contrast, dilatation of left ventricular cavity and reduced systolic function are the hallmarks of dilated cardiomyopathy (DCM). In 1991, it was reported for the first time that mutations in the gene encoding the βmyosin heavy chain, a major structural and contractile protein, was the genetic basis for familial hypertrophic cardiomyopathy associated with sudden death, ushering in the present era of cardiovascular genomic medicine. This seminal discovery permanently shifted the paradigm from the morphological to the molecular, enabling basic insights into disease pathogenesis to be viewed from how single gene defects orchestrate profound alterations at the biochemical, metabolic, hemodynamic, and physiological levels. In parallel, genetically engineered animals models became the state-of-the art for establishing causality and, ultimately, a basis to test proof-of-concept leading to disease prevention.

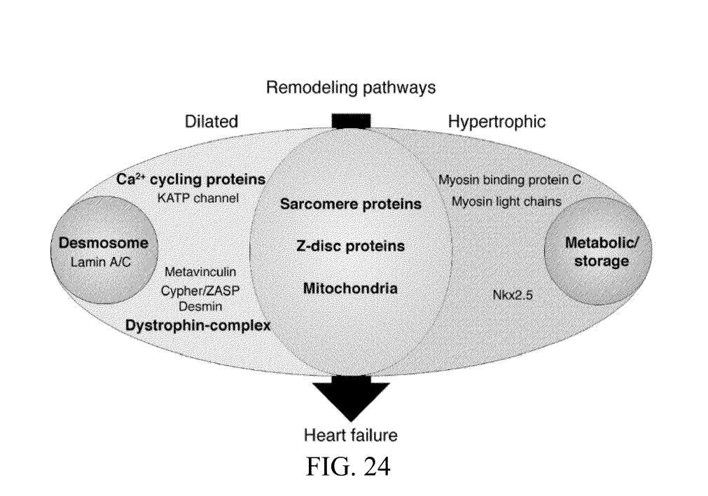

Many more single genetic defects are routinely being linked to familial heart failure (FIG. 24) but an important future challenge is to establish how inherited and acquired factors conspire to drive the growing epidemic of heart failure.

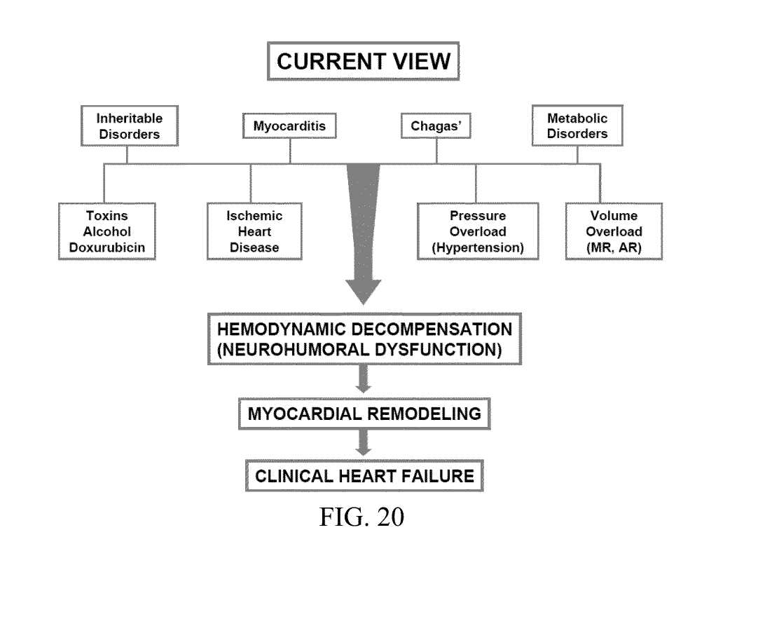

Severe occlusive coronary disease is the substrate for acute coronary syndromes, myocardial infarctions and subsequent pump failure as shown in FIG. 20. The high prevalence of heart failure in African-Americans with hypertension underscores potential gene-environment interactions in selected populations. Infectious etiologies (e.g., rheumatic heart disease) are declining but valvular heart disease from iatrogenic causes (e.g., diet pills, toxins) remains an important risk factor (FIG. 20). Viruses (e.g., Coxsackie's B3, parvovirus) are the major suspected culprits for idiopathic dilated cardiomyopathy (IDCM) in which the postviral sequalae of inflammation and apoptosis trigger ventricular remodeling and dilation (Liu and Mason, 2001). IDCM accounts for 30% of cases of dilated cardiomyopathy. Heart failure on presentation in the peri-partum or post-partum period has a variable clinical course from severe pump failure to complete recovery. The most common cause of right ventricular heart failure (RVHF) is left ventricular systolic dysfunction. In addition, RVHF is associated with congenital heart disease (e.g., tetralogy of fallot), primary pulmonary hypertension, and arrhythmogenic right ventricular dysphasia and right ventricular infarction. Stress cardiomyopathy is a rare reversible form of left ventricular dysfunction associated clinically with emotional stress, angiographically with ‘apical ballooning,’ and pathophysiologically with excess sympathetic activation (Wittstein et al., 2005). This entity remains a diagnosis of exclusion, which mimics ST segment elevation MI (STEMI) on presentation, has a much more favorable clinical outcome than STEM I. Lastly, thyrotoxicosis, Paget's disease and severe chronic anemia are rare causes of high output heart failure. Individuals afflicted with heart failure with preserved ejection fraction are more commonly older age, female gender and have a history of hypertension and atrial fibrillation.

d. Screening

The New York Heart Association (NYHA) functional classification scheme, an older but widely used screening tool, assesses the severity of functional limitations of individuals afflicted with heart failure. The four Classes of the NYHA classification are linked to increasing severity of signs and symptoms and correlate well with prognosis. This classification scheme, however, has important limitations since diverse pathophysiological processes leading to symptomatic heart failure are overlooked (Dunselman et al., 1988). Accordingly, the American College of Cardiology and American Heart Association (ACC/AHA) Classification of Chronic Heart Failure was developed to account for the multiple stages and predisposition conditions associated with the clinical syndrome. Designed to encompass emerging scientific evidence, an expert panel periodically assembles these updates, which are the most widely used and authoritative sources on the evaluation, management, performance measures and outcomes on heart failure (Bonow et al., 2005; Hunt et al., 2005; Radford et al., 2005). In turn, these guidelines incorporating preclinical stages, risk factors, pathophysiologic stages, and clinical recognition of heart failure are further subdivided into 4 stages. Stage A patients are at high risk for developing heart failure, but have had neither symptoms nor evidence of structural cardiac abnormalities. Major risk factors include hypertension, diabetes mellitus, coronary artery disease and family history of cardiomyopathy. In selected patients, the administration of angiotensin converting enzyme (ACE) inhibitor is recommended to prevent adverse ventricular remodeling.

Stage B patients have structural abnormalities from previous myocardial infarction, LV dysfunction or valvular heart disease but have remained asymptomatic. Both ACE inhibitors and beta-blockers are recommended.

Stage C patients have evidence for structural abnormalities along with current or previous symptoms of dyspnea, fatigue and impaired exercise tolerance. In addition to ACE inhibitors and beta-blockers, optimal medical regimen may include diuretics, digoxin, and aldosterone antagonists.

Stage D patients have end-stage symptoms of heart failure that are refractory to standard maximal medical therapy. Such patients are candidates for left ventricular assist devices and other sophisticated maneuvers for myocardial salvage or end-of-life care.

e. Pathophysiology

(A) Neurohumoral Mechanisms

Low cardiac output and systemic hypoperfusion elicit a cascade of compensatory mechanisms but predominantly activation of the neurohumoral pathway for augmenting fluid retention (FIG. 20). Sympathetic nervous system activation increases heart rate and peripheral vasoconstriction from the release of catecholamines, triggering increased afterload and myocardial oxygen consumption. Catecholamines also increase renin secretion, cell death, fibrosis, and myocardial irritability, underlying substrates for lethal arrhythmias and sudden death. In contrast, natriuretic peptides released from specialized cells in the atria exert hormonal actions in distant vascular beds, stimulating vasodilation and diuresis. Afterload reducing agents and beta-adrenergic blockers have significantly reduced the morbidity and mortality while improving the survival of patients with heart failure. Likewise, antagonists of aldosterone, which promotes salt and water retention, have proven clinical benefits.

(B) Myocardial Remodeling

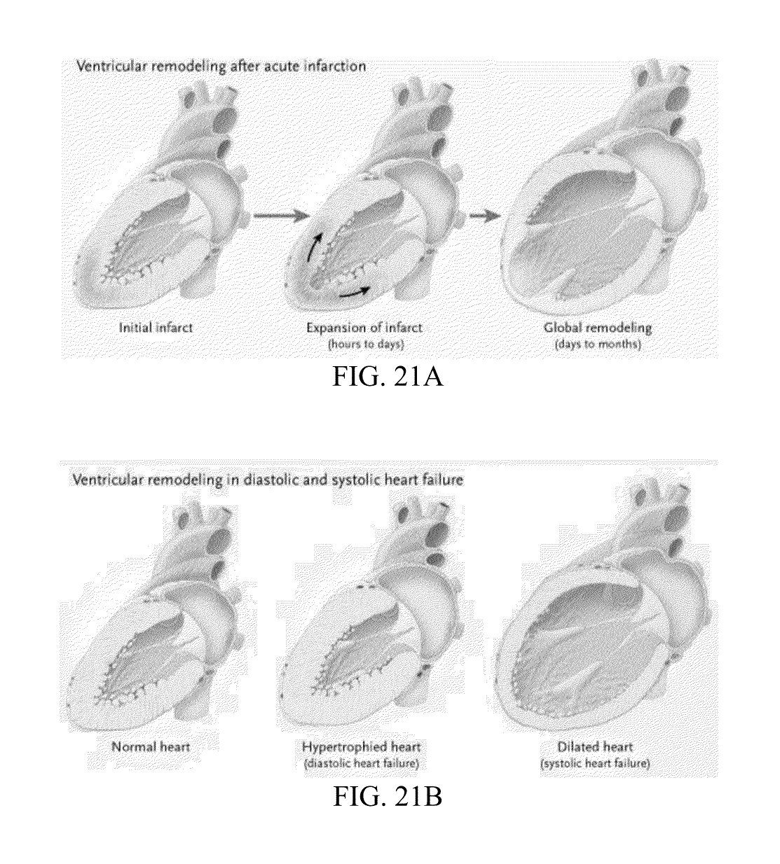

Left ventricular dysfunction and systolic heart failure secondary to myocardial infarction or ischemia are the prerequisites of low ejection fraction and elevated pulmonary pressures with congestion. Acquired or inherited conditions that either decrease cardiomyocyte viability and/or increase cell death will ultimately trigger pump failure and symptomatic heart failure. Given the heart's limited capacity for regeneration, terminally differentiated ventricular cardiomyocytes can undergo hypertrophy in response to increase metabolic and homodynamic demands. Activation of the ‘fetal gene program’ orchestrates transcriptional upregulation of genes encoding contractile and cytoskeletal proteins—the prerequisite for compensatory hypertrophy. Recruitment of such adaptive mechanisms provides a variable but stable and asymptomatic interval—perhaps lasting years—before cardiac decompensation. The ensuing ventricular dilatation is a pathologic form of adaptation, termed ‘ventricular remodeling,’ affecting intrinsic cardiac mass, the extracellular matrix, collagen deposition and fibrosis as shown in FIG. 20 and FIG. 21. Whereas low levels of reactive oxygen species (ROS) serve as stress signals in redox-dependent regulation, elevated levels of ROS caused by mitochondrial dysfunction may alter myocardial energetics, cardiac metabolism, and trigger the release of cytochrome c, thereby activating cell survival/death pathways. Endothelial dysfunction gives rise to the aberrant release of nitric oxide, a potent vasodilator, and/or reactivity with reactive oxygen species to form peroxynitrite, which causes oxidative damage and cellular injury. Progressive remodeling, in attempts to maintain systolic function and homeostasis (Stage B), leads to valvular regurgitation from inadequate apposition of the mitral leaflets, increasing myocardial stress and, ultimately, decompensated heart failure (Stage C and Stage D).

(C) Mechanisms of Cell Death in Heart Failure

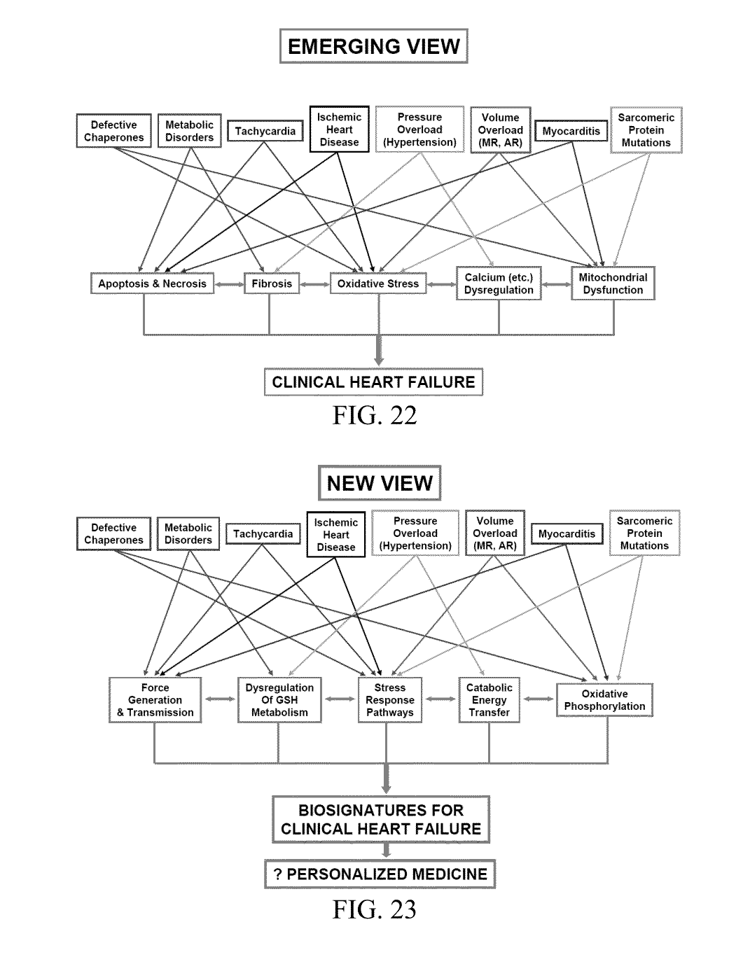

Progressive loss of cardiomyocytes from either necrosis or apoptosis with diverse pathogenic states contributes to the pathogenesis of heart failure as shown in Table 2 and FIG. 22 (Liew and Dzau, 2004; Wencker et al., 2003). Apoptosis or programmed cell death is activated by signaling cascades, via either the extrinsic or intrinsic cell survival/death pathways (Danial and Korsmeyer, 2004). Ligands such as TNF-α, which bind to cognate receptors at the plasma membrane, mediate cell death through the extrinsic pathway, whereas the Bcl-2 family—consisting of both pro- and anti-apoptotic proteins—regulates the intrinsic pathway. Mitochondria play a central role in cell survival/death principally from the initiation of stress signals (e.g., reactive oxygen species) and release of mitochondrial cytochrome c, which initiates complex formation and the activation of apoptotic proteases (e.g., caspase-9) (Danial and Korsmeyer, 2004). The role of apoptosis in chronic heart failure, which ranges between 80-250 myocytes per 100,000 nuclei in failing human hearts was elegantly validated by Wencker and coworkers using transgenic mice harboring a fusion protein FKBP fused with a conditionally active caspase (Wencker et al., 2003). In contrast, low-level inhibition of apoptosis prevented dilated cardiomyopathy and death, suggesting possible therapeutic strategies for combating heart failure.

TABLE 2
The Genetic Basis of Cardiomyopathies

| Symbol | Chromosome | Gene Product | Cardiomyopathy Type |
|---|---|---|---|
| ACTC | 15q11-14 | Cardiac muscle α-actin | Hypertrophic and dilated |
| ABCC9 | 12p12.1 | Member 9 of the superfamily C of | Dilated |
| | | ATP-binding cassette (ABC) | |
| | | transporters | |
| CSRP3 | 11p15.1 | Cysteine-and glycine-rich protein 3 | Dilated |
| MLP | | | |
| DES | 2q35 | Desmin | Dilated |
| DSP | 6p24 | Desmoplakin | |
| LMNA | 1q21.2-21.3 | Lamin A/C | Dilated |
| VCL | 10q22.1-q23 | Metavinculin | Dilated |
| MYBPC3 | 11p11.2 | Cardiac myosin-binding protein C | Hypertrophic and dilated |
| MYH6 | 14q12 | Cardiac muscle α-isoform of | Hypertrophic |
| | | myosin heavy chain (heavy | |
| | | polypeptide 6) | |
| MYH7 | 14q12 | Cardiac muscle α-isoform of | Hypertrophic and dilated |
| | | myosin heavy chain (heavy | |
| | | polypeptide 7) | |
| MYL2 | 12q23-24.3 | Myosin regulatory light chain | Hypertrophic |
| | | associated with cardiac myosin-β | |
| | | (or slow heavy chain) | |
| MYL3 | 3p21.2-21.3 | Myosin light chain 3 | Hypertrophic |
| PLN | 6q22.1 | Phospholamban | Dilated |
| PRKAG2 | 7q35-36 | γ2 non-catalytic subunit of AMP- | |
| | | activated protein kinase | |
| SGCB | 4q12 | β-Sarcoglycan (43 kDa dystrophin- | Dilated |
| | | associated glycoprotein) | |
| SGCD | 5q33-34 | δ-Sarcoglycan (35 kDA dystrophin- | Dilated |
| | | associated glycoprotein) | |
| TAZ, | Xq28 | Tafazzin | Dilated |
| G4.5 | | | |
| TTN | 2q31 | Titin | Hypertrophic and dilated |
| TCAP | 17q12 | Titin-cap | Dilated |
| TPM1 | 15q22.1 | Tropomyosin 1 (α) | Hypertrophic |
| TNNI3 | 19q13.4 | Troponin 1, a subunit of the | |
| | | troponin complex of the thin | |
| | | filaments of striated muscle | |
| TNNT2 | 1q32 | Cardiac isoform of troponin T2, | Hypertrophic and dilated |
| | | tropomyosin-binding subunit of the | |
| | | troponin complex | |

f. Diagnosis

Newly diagnosed patients with heart failure most commonly seek medical attention for either gradual or abrupt onset of the classical signs and symptoms with pulmonary congestion. The clinical spectrum varies widely but dyspnea on exertion, peripheral edema, orthopnea, and paroxysmal nocturnal dyspnea are not uncommon. Exertional chest pain or angina at rest requires an immediate evaluation to determine if biochemical evidence of myocardial damage demands more aggressive management for acute coronary syndromes. Elevated jugular venous distension from right ventricular failure, ascites, and cachexia are more ominous signs for low cardiac output and decompensation, requiring urgent attention, preferably, from a provider who specializes in heart failure management.

Routine diagnostic studies include an electrocardiogram, chest radiograph, and B-type natriuretic peptide, the latter having the best predictive value for distinguishing between CHF and non-CHF patients (Maisel and McCullough, 2003). Noninvasive echocardiography is the most commonly used diagnostic tool for the assessment and follow-up of patients with heart failure with or without preserved ejection fraction. Coronary angiography should be performed to exclude reversible causes for left ventricular dysfunction or to guide prompt revascularization. If the coronary vessels are widely patent in the setting of global dysfunction, then endomyocardial biopsy should be considered to assess for reversible causes including viral myocarditis (Liu and Mason, 2001). Equilibrium radionucleotide angiography (ERNA) is another noninvasive diagnostic study that assesses both left and right ventricular systolic function. Screening tools such as contrast computer tomographic angiography and magnetic resonance imaging (MRI) are gaining attention as emerging technologies with equivalent sensitivity and specificity as the invasive angiogram for coronary arteriography. MRI may also uncover unsuspected infiltrative cardiomyopathy, arryhythmogenic right ventricular dysplasia, and is superior for the assessment of myocardial viability before revascularization.

g. Prognosis

Over 5 million Americans or 1.5% of the US population have chronic heart failure, and there is a similar prevalence at risk of undiagnosed left ventricular dysfunction (Braunwald and Bristow, 2000). With over 550,000 new cases of heart failure, the disproportionate health and economic burden exceeds 24 billion dollars annually (DiBianco, 2003). Soon, heart failure will become the number one cause of death worldwide, eclipsing infectious disease (Bleumink et al., 2004). Whereas new pharmacologic management and revascularization techniques continue to improve the survival after acute myocardial infarction, the prevalence of chronic heart failure appears to be increasing as the population ages (Braunwald and Bristow, 2000). Notwithstanding, heart failure accounts for 20 percent of all hospital admissions in patients older than 65, and the hospitalization rate has increased by 159 percent in the past decade (Jessup and Brozena, 2003). Available treatments for heart failure have only modestly improved the morbidity and mortality (Jessup and Brozena, 2003), and for patients with advanced heart failure, the prognosis still remains grim with 1-year mortality rates between 20-45%, overshadowing the worse forms of some cancers (Jessup and Brozena, 2003).

h. Pharmocogenomics

Substantial phenotypic heterogeneity in heart failure and the variability of responses among individuals taking pharmacologic agents have been attributed to common polymorphisms in the genome (Liggett, 2001). A surmountable hurdle, however, is the robustness of the association between putative genetic markers and therapeutic response. Recent lessons from studies of human heart failure illustrate handsomely both the enormous potential and challenges for pharmacogenomics—a maturing discipline in which an individual's genetic determinants are used to predict drug response and outcomes (Evans and Relling, 1999; Liggett, 2001). It has been demonstrated that non-synonymous single-nucleotide polymorphisms of the β1-adrenergic receptor (β1-AR), a member of the seven membrane-spanning receptor superfamily, alters the therapeutic response to â-blockers during heart failure (Liggett et al., 2006).

Stimulatory effects between β1-AR and heterotrimeric G proteins: Gs mediate both beneficial and deleterious signal transduction pathways during the onset and progression of heart failure. Because β1-AR is the major subtype in cardiac myocytes, increased catecholamines exert potent cardiomyopathic effects, cardiac remodeling and abrogation of gene expression, which are antagonized by β1-AR blockers resulting in improved outcomes. A single nucleotide variation at nucleotide 1165 in the gene encoding β1-AR results in either Arg or Gly at position 389 residue (Liggett et al., 2006). In response to inotropic stimulation, human trabeculae muscle with the 131-Arg-389 residue from either nonfailing or failing hearts exhibited significantly greater contractility than 131-Gly-389 polymorphism.

As shown in the β-Blocker Evaluation of Survival Trial (BEST), which evaluated the β-Blocker bucindolol for the treatment of Class III/IV, insights into the mechanisms for pharmacogenomic phenotypes involving the Arg/Gly polymorphism of the β1-AR owe much credit to the DNA Study Group (Feldman et al., 2005; Liggett et al., 2006). The foresight of BEST investigators to recognize the power of genetic haplotyping underscores the importance for all future well-designed human trials to include contingencies for pharmacogenomics in an era of genomic medicine. Notwithstanding the success gleaned from a highly penetrant single-gene trait such as Arg/Gly polymorphism of the β-AR, future advances will require undertaking the more formidable challenges related to multigene traits that influence drug metabolism and response for therapeutic individualization (Evans and McLeod, 2003).

The recent African-American Heart Failure (A-HeFT) enrolled 1050 black patients with NYHA class III or IV to receive a fixed dose of two well established medications, isosorbide dinitrate and hydralazine, in a placebo controlled randomized multicenter trial. Combination nitrates and hydralazine, termed BiDil, when added to standard therapy was efficacious improving survival in blacks. But the implications of this high-profile study have drawn considerable scientific and ethical scrutiny owing to the marketing strategy of this therapy, under the proprietary label, was advanced as a novel approach for race-based management. Because physical and genetic traits are not interchangeable, AHeFT per se might prove to be poor surrogate for studies of pharmacogenetics since neither BiDil's efficacy in outer racial and ethnic groups nor genetic markers for predicting the response of blacks to BiDil were ever tested.

In contrast, polymorphisms in the angiotensin converting enzyme (ACE) pathway has been extensively studied especially the ACE DD polymorphism, which had significantly higher death and need for transplant compared to II and ID genotypes (McNamara et al., 2001). With concurrent β-blocker treatment, patients with ACE DD polymorphism showed improved survival but benefited with a higher ACE dosage (McNamara et al., 2004), supporting the clinical utility of genetic information in clinical management. For at-risk populations, the pace for moving bidirectional bench [symbol] beside and beside [symbol] community-based practices should accelerate using evidence-based strategies emerging from disciplines such as health outcomes.

ii. Cardiomyopathy

Cardiomyopathy, which literally means “heart muscle disease”, is the deterioration of the function of the myocardium (i.e., the actual heart muscle) for any reason. People with cardiomyopathy are often at risk of heart failure. Thus, the disclosed method can be used to treat a subject with a cardiomyopathy. Cardiomyopathies can generally be categorized into two groups, based on World Health Organization guidelines: extrinsic cardiomyopathies and intrinsic cardiomyopathies.

Extrinsic cardiomyopathies are cardiomyopathies where the primary pathology is outside the myocardium itself. Most cardiomyopathies are extrinsic, because by far the most common cause of a cardiomyopathy is ischemia. The World Health Organization calls these specific cardiomyopathies ischemic (or ischaemic) cardiomyopathy, hypertensive cardiomyopathy, valvular cardiomyopathy, inflammatory cardiomyopathy, cardiomyopathy secondary to a systemic disease, and alcoholic cardiomyopathy.

Ischemic cardiomyopathy is a weakness in the muscle of the heart due to inadequate oxygen delivery to the myocardium with coronary artery disease being the most common cause. Individuals with ischemic cardiomyopathy typically have a history of myocardial infarction (heart attack), although longstanding ischemia can cause enough damage to the myocardium to precipitate a clinically significant cardiomyopathy even in the absence of myocardial infarction. In a typical presentation, the area of the heart affected by a myocardial infarction will initially become necrotic as it dies, and will then be replaced by scar tissue (fibrosis). This fibrotic tissue is akinetic; it is no longer muscle and cannot contribute to the heart's function as a pump. If this akinetic region of the heart is substantial enough, the affected side of the heart (i.e. the left or right side) will go into failure, and this failure is the functional result of an ischemic cardiomyopathy.

Many diseases can result in cardiomyopathy. These include diseases like hemochromatosis, (an abnormal accumulation of iron in the liver and other organs), amyloidosis (an abnormal accumulation of the amyloid protein), diabetes, hyperthyroidism, lysosomal storage diseases and the muscular dystrophies.

An intrinsic cardiomyopathy is weakness in the muscle of the heart that is not due to an identifiable external cause. Intrinsic cardiomyopathy has a number of causes including drug and alcohol toxicity, certain infections (including Hepatitis C), and various genetic and idiopathic (i.e., unknown) causes. There are four main types of intrinsic cardiomyopathy. Dilated cardiomyopathy (DCM) is the most common form, and one of the leading indications for heart transplantation. In DCM the heart (especially the left ventricle) is enlarged and the pumping function is diminished. Hypertrophic cardiomyopathy (HCM or HOCM) is a genetic disorder caused by various mutations in genes encoding sarcomeric proteins. In HCM the heart muscle is thickened, which can obstruct blood flow and prevent the heart from functioning properly. Arrhythmogenic right ventricular cardiomyopathy (ARVC) arises from an electrical disturbance of the heart in which heart muscle is replaced by fibrous scar tissue. The right ventricle is generally most affected. Restrictive cardiomyopathy (RCM) is the least common cardiomyopathy where the walls of the ventricles are stiff, but may not be thickened, and resist the normal filling of the heart with blood. A rare form of restrictive cardiomyopathy is the obliterative cardiomyopathy, seen in the hypereosinophilic syndrome. In this type of cardiomyopathy, the myocardium in the apicies of the left and right ventricles become thickened and fibrotic, causing a decrease in the volumes of the ventricles and a type of restrictive cardiomyopathy.

The disclosed method can be used to treat a subject with left ventricular hypertrophy (HCM or HOCM).

The disclosed method can further be used to treat a subject with protein aggregation cardiomyopathy. Thus, the subject can comprise a mutation in αB-crystallin (CryAB) or desmin. For example, the subject can comprise a R120G mutation in CryAB (R120GCryAB).

iii. G6PD Inhibitor

Dehydroepiandrosterone (DHEA) and DHEA-sulfate are major adrenal secretory products in humans. The plasma concentration of DHEA-sulfate, which next to cholesterol, is the most abundant steroid in humans, undergoes the most marked age-related decline of any known steroid.

Although DHEA-sulfate is the main precursor of placental estrogen and may be converted into active androgens in peripheral tissue, there is no obvious biological role for either DHEA or DHEA-sulfate in the normal individual. However, it has been established that DHEA is a potent non-competitive inhibitor of mammalian glucose-6-phosphate dehydrogenase (G6PDH). For example, see Oertel, et al., “The effects of steroids on glucose-6-phosphate dehydrogenase,” J. Steroid Biochem., 3, 493-496 (1972) and Marks, et al., “Inhibition of mammalian glucose-6-phosphate dehydrogenase by steroids,” Proc. Nat'l Acad. Sci, U.S.A., 46, 477-452 (1960).

Thus, the inhibitor of G6PD can be Dehydroepiandrosterone (DHEA) or DHEA-sulfate (DHEA-S). However, there is however evidence of an estrogenic effect after prolonged administration of DHEA, which is not an estrogen per se but is well known to be convertible into estrogens. In addition, the therapeutic dose of DHEA is rather high. Thus, the inhibitor of G6PD can be an analogue of DHEA.

For example, 16α-bromoepiandrosterone is a more potent inhibitor of mammalian G6PDH than DHEA (Schwartz, et al. 1981. Carcinogensis, Vol. 2 No. 7, 683-686). Thus, the inhibitor of G6PD can be 16α-bromoepiandrosterone (EPI).

U.S. Pat. Nos. 5,001,119 and 5,700,793 are incorporated herein by reference for the teaching of DHEA analogues and methods of making and administering same. For example, the inhibitor of G6PD can be 16α-hydroxy-5-androsten-17-one, 16α-fluoro-5-androsten-17-one (fluasterone), 16α-fluoro-16β-methyl-5-androsten-17-one, 16α-methyl-5-androsten-17-one, 16β-methyl-5-androsten-17-one, 16α-hydroxy-5α-androstan-17-one, 16α-fluoro-5α-androstan-17-one, 16α-fluoro-16β-methyl-5α-androstan-17-one, 16α-methyl-5α-androstan-17-one, or 16β-methyl-5α-androstan-17-one.

16α-fluoro-5-androsten-17-one (fluasterone) is a synthetically stable adrenocortical steroid analogue of DHEA. Fluasterone has consistently and repeatedly shown superior efficacy to DHEA while simultaneously limiting side effects. Thus, the inhibitor of G6PD can be 16 alpha-fluoro-5-androsten-17-one (fluasterone).

### 2. Method of Treating Reductive Stress

Disclosed herein is a causative link mechanism for ‘reductive stress’ in the pathogenesis of hR120G-induced cardiomyopathy. This is the first such report of reductive stress being involved in disease pathology. However, as disclosed herein, reductive stress has played an unappreciated role in other pathologies and conditions. Thus, provided herein is a method of treating or preventing a condition in a subject caused or exacerbated by reductive stress, comprising administering to the subject a therapeutically effective amount of a composition comprising an anti-reductant (i.e., pro-oxidant) molecule. Also provided is a method of treating or preventing a condition in a subject caused or exacerbated by reductive stress, comprising: diagnosing a subject as having or at risk of having said condition, and administering to the subject a composition comprising an anti-reductant molecule.

i. Redox

Redox reactions include all chemical processes in which atoms have their oxidation number (oxidation state) changed. This can be a simple redox process, such as the oxidation of carbon to yield carbon dioxide, it could be the reduction of carbon by hydrogen to yield methane (CH₄), or a complex process such as the oxidation of sugar in the human body, through a series of very complex electron transfer processes. The term redox comes from the two concepts of reduction and oxidation. It can be explained in simple terms: oxidation describes the loss of an electron by a molecule, atom or ion, and reduction describes the gain of an electron by a molecule, atom or ion. However, these descriptions (though sufficient for many purposes) are not truly correct. Oxidation and reduction properly refer to a change in oxidation number—the actual transfer of electrons may never occur. Thus, oxidation is better defined as an increase in oxidation number, and reduction as a decrease in oxidation number. In practice, the transfer of electrons will always cause a change in oxidation number, but there are many reactions that are classed as “redox” even though no electron transfer occurs (such as those involving covalent bonds).

ii. Oxidizing and Reducing Agents

Substances that have the ability to oxidize other substances are said to be oxidative and are known as oxidizing agents, oxidants or oxidizers. Put in another way, the oxidant removes electrons from another substance, and is thus reduced itself And because it “accepts” electrons it is also called an electron acceptor. Substances that have the ability to reduce other substances are said to be reductive and are known as reducing agents, reductants, or reducers. Put in another way, the reductant transfers electrons to another substance, and is thus oxidized itself. And because it “donates” electrons it is also called an electron donor.

Much biological energy is stored and released by means of redox reactions. Photosynthesis involves the reduction of carbon dioxide into sugars and the oxidation of water into molecular oxygen. The reverse reaction, respiration, oxidizes sugars to produce carbon dioxide and water. As intermediate steps, the reduced carbon compounds are used to reduce nicotinamide adenine dinucleotide (NAD+), which then contributes to the creation of a proton gradient, which drives the synthesis of adenosine triphosphate (ATP) and is maintained by the reduction of oxygen. In animal cells, mitochondria perform similar functions.

The term redox state is often used to describe the balance of NAD+/NADH and NADP+/NADPH in a biological system such as a cell or organ. The redox state is reflected in the balance of several sets of metabolites (e.g., lactate and pyruvate, beta-hydroxybutyrate and acetoacetate) whose interconversion is dependent on these ratios. An abnormal redox state can develop in a variety of deleterious situations, such as hypoxia, shock, and sepsis. Redox signaling involves the control of cellular processes by redox processes.

iii. Reductive Stress Conditions

The herein disclosed methods can be used to treat or prevent any condition known or newly discovered to be caused or exacerbated by reductive stress. One of skill in the art can ascertain whether a specific condition involves reductive stress using known biomarkers and assays, some of which are disclosed herein. For example, in some aspects, the condition of the disclosed method is characterized by increased levels of reduced glutathione (GSH) and/or an increase in the ratio of GSH to oxidized glutathione (GSSG) in a tissue or cell of the subject.

In some aspects, the condition of the disclosed method is characterized by increased levels of reduced nicotinamide adenine dinucleotide phosphate (NADPH) and/or an increase in the ratio of NADPH to oxidized nicotinamide adenine dinucleotide phosphate (NADP+) in a tissue or cell of the subject.

In some aspects, the condition of the disclosed method is characterized by increased levels of heat shock protein 25/27 (HSPB1; also known as heat shock protein (Hsp25) and heat shock protein 27 (Hsp27)). Hsp25 overexpression increases GSH content and confers oxidative resistance (Mehlen P, et al. 1996; Baek S H, et al. 2000), whereas Hsp25 down-regulation, linked to GSH depletion, increases oxidative stress (Christians E S, et al. 2002; Yan L J et al. 2002). Thus, as disclosed herein, Hsp25 can in some aspects stimulate G6PD activity.

In some aspects, the condition of the disclosed method is diabetes. Diabetes mellitus is a metabolic disorder characterized by hyperglycemia (high glucose blood sugar), among other signs. The World Health Organization recognizes three main forms of diabetes: type 1, type 2 and gestational diabetes (or type 3, occurring during pregnancy), although these share signs and symptoms but have different causes and population distributions. Type 1 is generally due to autoimmune destruction of the insulin-producing cells—pancreatic beta cells—while type 2 is characterized by tissue wide insulin resistance and varies widely. Gestational diabetes is due to a poorly understood interaction between fetal needs and maternal metabolic controls. Type 2 sometimes progresses to loss of beta cell function as well.

In some aspects, the condition of the disclosed method is an acute coronary syndrome appropriate for percutaneous coronary interventions (PCI). Percutaneous coronary intervention (PCI), commonly known as coronary angioplasty, is an invasive cardiologic therapeutic procedure to treat the stenotic (narrowed) coronary arteries of the heart. These stenotic segments are due to the build up of cholesterol-laden plaques that form due to coronary heart disease. Percutaneous coronary intervention can be performed to reduced or eliminate the symptoms of coronary artery disease, including angina (chest pain), dyspnea (shortness of breath) on exertion, and congestive heart failure. PCI is also used to abort an acute myocardial infarction, and in some specific cases it may reduce mortality.

In some aspects, the condition of the disclosed method is an acute coronary syndrome. An acute coronary syndrome (ACS) is a set of signs and symptoms suggestive of sudden cardiac ischemia, usually caused by disruption of atherosclerotic plaque in an epicardial coronary artery. The acute coronary syndromes include Unstable Angina (UA), Non-ST Segment Elevation Myocardial Infarction (NSTEMI), and ST Segment Elevation Myocardial Infarction (STEMI), commonly referred to as a heart attack. Thus, in some aspects, the condition of the disclosed method is acute myocardial infarction.

In some aspects, the condition of the disclosed method is an acute brain attack (stroke). A stroke, also known as cerebrovascular accident (CVA), is an acute neurological injury in which the blood supply to a part of the brain is interrupted. That is, a stroke involves the sudden loss of neuronal function due to disturbance in cerebral perfusion. This disturbance in perfusion is commonly arterial, but can be venous.

In some aspects, the condition of the disclosed method is cardiac hypertrophy, cardiomyopathy, and/or heart failure. Thus, in some aspects, the condition of the disclosed method is protein aggregation cardiomyopathy. Thus, the subject of the disclosed method can comprise a mutation in αB-crystallin (CryAB) or desmin. For example, the subject can comprise a R120G mutation in CryAB (R120GCryAB).

iv. Anti-Reductant

a. Thiuram Disulfide

Thus, the anti-reductant molecule of the disclosed method can be a dithiocarbamate, thiuram disulfide, such as a tetrathiuram disulfide, such as tetraalkylthiuram disulfide (disulfuram), or a thiocarbamate-metal complex. In some aspects, the anti-reductant molecule of the disclosed method does not comprise disulfuram. In some aspects, the anti-reductant molecule of the disclosed method does not comprise sodium selenite.

As used herein, the term “thiuram disulfide” refers to compounds having the formula of:

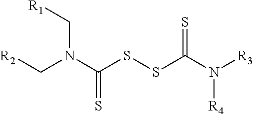

where R₁, R₂, R₃, and R₄ are same or different and represent hydrogen, and unsubstituted or substituted alkyl, alkenyl, alkynyl, aryl, alkoxy, and heteroaryl groups. It is noted that the alkyl groups can include cycloalkyl and heterocycloalkyl groups. R₁, R₂, and the N atom in the formula can together form an N-heterocyclic ring, which is, e.g., heterocycloalkyl or heterocycloaryl. Likewise, R₃, and R₄ and the N atom in the formula can together form an N-heterocyclic ring, which is, e.g., heterocycloalkyl or heterocycloaryl. Typically, R₁ and R₂, are not both hydrogen, and R₃, and R₄ are not both hydrogen. Thus, thiuram disulfide is a disulfide form of dithiocarbamates which have a reduced sulfhydryl group. Many dithiocarbamates are known and synthesized in the art. Nonlimiting examples of dithiocarbamates include diethyldithiocarbamate, pyrrolidinedithiocarbamate, N-methyl, N-ethyldithiocarbamates, hexamethylenedithiocarbamate, imadazolinedithiocarbamates, dibenzyldithiocarbamate, dimethylenedithiocarbamate, dipopyldithiocarbamate, dibutyldithiocarbamate, diamyldithiocarbamate, N-methyl, N-cyclopropylmethyldithiocarbamate, cyclohexylamyldithiocarbamate pentamethylenedithiocarbamate, dihydroxyethyldithiocarbamate, N-methylglucosamine dithiocarbamate, and salts and derivatives thereof. Typically, a sulfhydryl-containing dithiocarbamate can be oxidized to form a thiuram disulfide.

Any pharmaceutically acceptable form of thiuram disulfides as defined above can be used. For example, tetraalkylthiuram disulfide, which is known as disulfuram, can be used in the disclosed method. Disulfuram has the following formula:

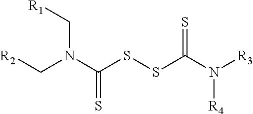

where R₁, R₂, R₃, and R₄ are all ethyl. Disulfuram has been used clinically in the treatment of alcohol abuse, in which disulfuram inhibits hepatic aldehyde dehydrogenase. Methods of making thiuram disulfides are generally known in the art. Exemplary methods are disclosed in, e.g., Thorn, et al., The Dithiocarbamates and Related Compounds, Elsevier, N.Y., 1962; and U.S. Pat. Nos. 5,166,387, 4,144,272, 4,066,697, 1,782,111, and 1,796,977, all of which are incorporated herein by reference.

U.S. Pat. Nos. 6,589,987, 6,706,759, and 6,548,540 are incorporated by reference herein for the teaching of disulfuram and methods of making and administering same. Many dithiocarbamates are known and synthesized in the art. Non-limiting examples of dithiocarbamates include diethyldithiocarbamate (DEDTC), pyrrolodinedithiocarbamate, N-methyl, N-ethyl dithiocarbamates, hexamethylenedithiocarbamate, imidazolinedithiocarbamates, dibenzyldithiocarbamate, dimethylenedithiocarbamate, dipolyldithiocarbamate, dibutyldithiocarbamate, diamyldithiocarbamate, N-methyl, N-cyclopropylmethyldithiocarbamate, cyclohexylamyldithiocarbamate, pentamethylenedithiocarbamate, dihydroxyethyldithiocarbamate, N-methylglucosamine dithiocarbamate, and salts and derivatives thereof. Typically, a sulfhydryl-containing dithiocarbamate can be oxidized to form a dithiocarbamate disulfide.

The dithiocarbamates comprise a broad class of molecules giving them the ability to complex metals and react with sulfhydryl groups and glutathione. In addition to their reduced thioacid form, dithiocarbamates exist in three other forms, e.g., a) the disulfide, a condensed dimmer of the thioacid, with elimination of reduced sulfhydryl groups by disulfide bond formation; b) the negatively charged thiolate anion, generally as the alkali metal salt, such as sodium; and c) the 1,1-dithiolato complexes of the transition elements, in which the two adjoining sulfur atoms of the dithiocarbamate are bound to the same titanium, vanadium, chromium, iron, cobalt, nickel, copper, silver or gold metal ion.

Heavy metal ions such as copper, zinc, gold, and silver ions can significantly enhance the effect of thiuram disulfides, while the depletion of such heavy metal ions can prevent effect of disulfuram. Accordingly, in some aspects of the disclosed method, the anti-reductant molecule of the disclosed method is a thiuram disulfide and a heavy metal ion. Non-limiting examples of heavy metal ions include ions of arsenic, bismuth, cobalt, copper, chromium, gallium, gold, iron, manganese, nickel, silver, titanium, vanadium, selenium and zinc. Sources of such heavy metal ions are known to the ordinary artisan. For example, such ions can be provided in a sulfate salt, or chloride salt form, or any other pharmaceutically suitable forms.

One or more thiuram disulfide compounds and one or more heavy metal ions can be administered to a patient. The thiuram disulfide compound and the heavy metal ion can be administered in combination or separately. For example, they can be administered as a chelating complex. As is known in the art, thiuram disulfide compounds are excellent chelating agents and can chelate heavy metal ions to form chelates. Preparation of chelates of thiuram disulfide compounds and heavy metal ions are known to the ordinary artisan. For example, chelates of disulfuram and copper, zinc, silver, or gold ions can be conveniently synthesized by mixing, in a suitable solvents, disulfuram with, e.g., CuSO₄, ZnCl₂, C₃H₅AgO₃, or HAuCl₄3H₂O to allow chelates to be formed. Other thiuram disulfide compound-heavy metal ion chelates are disclosed in, e.g., Burns et al., Adv. Inorg. Chem. Radiochem. 23:211-280 (1980), which is incorporated herein by reference.

In some aspects, the anti-reductant molecule of the disclosed method is a thiuram disulfide compound and an intracellular heavy metal ion stimulant, which can enhance the intracellular level of the above described heavy metal ions in the patient. Intracellular heavy metal ion carriers are known. For example, ceruloplasmin can be administered to the patient to enhance the intracellular copper level. Other heavy metal ion carriers known in the art may also be administered in accordance with this aspect of the invention. The heavy metal ion carriers and the thiuram disulfide compound can be administered together or separately, and preferably in separate compositions.

U.S. Patent Publication No. 2005/0096304 is incorporated by reference herein for the teaching of thiocarbamate metal complexes and methods of making and administering same. For example, sulfhydryl-containing dithiocarbamates can be converted to their corresponding thiolate anions by treatment with an alkali-metal hydroxide as a proton acceptor. The metal ion coordination compounds of dithiocarbamates can be synthesized either by treatment of the disulfide or the thiolate anion forms of dithiocarbamates with metal ion sources yielding a variety of useful metal compounds in which the dithiocarbamate is a bidentate ligand to the same metal ion.

b. G6PD Inhibitor

Thus, the anti-reductant molecule of the disclosed method can be an inhibitor of glucose-6-phosphate dehydrogenase (G6PD). Examples of G6PD inhibitors are provided below. However, other known or newly discovered inhibitors of G6PD with anti-reductant properties can be used as in the disclosed methods.

c. Cysteine-Rich Protein

Human serum albumin prevents hydroxy radical-induced aggregation of human fibrinogen iin vitro (Lipinski B. 2002). The reducing potential of hydroxyl radical on fibrinogen aggregation is inhibited by human serum albumin, supporting its role as an antireductant rather than an antioxidant. Thirty-four out of thirty-five cysteines of human serum albumin exist as oxidized disulfides. Thus, the anti-reductant molecule of the disclosed method can be a protein comprising at least 10 cystein residues, wherein at least 90% of the cystein residues comprise oxidized disulfides. For example, the protein of the method can be a serum albumin, such as human serum albumin.

d. Biomolecule Comprising Electrophilic Methyl Groups

Biomolecules containing charged nitrogen or sulfur atoms bound to a methyl group (termed electrophilic methyl groups) can react with NADH and, thereby, ameliorate reductive stress (Ghyczy M, et al. 2001). Thus, the anti-reductant molecule of the disclosed method can be a biomolecule comprising a charged nitrogen or sulfur atom linked to a methyl group.

v. Pharmaceutical Carriers

The disclosed compositions can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### 3. Diagnosing and Monitoring Heart Failure

i. Genomic Profiling

Heart failure encompasses dynamic processes in which the activation or deactivation of distinct pathways at different stages in the pathogenesis indicates opportunities for intervention and even prevention before irreversible decompensation. A fundamental question, therefore, is how to develop improved diagnostic and prognostic indices that can guide improvements in treatment and outcomes for heart failure. A major goal of microarray-based analyses is to identify genes whose similar patterns of expression accurately represent the disease state or biological process. Such information, however, is often insufficient to identify the causal mechanisms but provides a comprehensive picture of the underlying process, which can predict responses to therapy or disease stage.

Both unsupervised and supervised approaches are applied to determine if previously unrecognized or unexpected patterns of expression exist in the datasets. Hierarchical clustering, for example, is an unsupervised approach that may be used after gene expression profiling to identify interdependent pathways before the onset of overt heart failure. Identification and validation of genes or novel pathways that are activated earliest may improve early detection and, ultimately, will be essential for designing therapies that prevent the natural history and progression of disease. If individual genes have different predictive power, then a ‘weighted voting scheme,’ based on the levels of gene expression, can be designed and tested before widespread application.

ii. Transcriptional Profiling of Heart Failure

Neurohumoral, hemodynamic and environmental factors participate in remodeling the failing heart, but genetic, molecular and cellular events are inscribed at the transcriptional level. Signaling pathways and biological processes implicated in the hypertrophic response of the heart are shown in FIG. 22. Early genetic markers of cardiac hypertrophy include transcriptional reprogramming of genes encoding contractile proteins, oncogenes, neurohumoral factors and transcription factors have been identified. Genes encoding proto-oncogenes c-jun, c-fos, c-myc, skeletal α-actin and ANF are also activated in response to hypertrophic stimuli. (Izumo et al., 1988; Komuro et al., 1988; Mulvagh et al., 1987; Schwartz et al., 1986). In angiotensin II receptor type 1 alpha knockout mice, cardiomyocytes were capable of evoking increased protein synthesis and MAPK activation when stretched, strengthening the primary role of mechanical stretch in maintaining the hypertrophic phenotype. The mechanisms by which mechanical stress is converted into biological response are yet to be fully elucidated. High-density oligonucleotide arrays have also identified multiple genes, representing diverse biological process (e.g. myocardial structure, myocardial assembly and degradation, metabolism, protein synthesis and stress response), which were differentially expressed in nonfailing and failing human hearts (Yang et al., 2000). Other larger studies of human heart failure have confirmed the role for mitogen-activated protein kinases (MAPKs), mechanical stress and neurohumoral pathways in heart failure (Kudoh et al., 1998). Likewise, genetic pathways identified during acute and chronic pressure overload reflect differential gene expression during distinct phases may represent potential target for therapy. A substantial limitation, however, remains that lack of reproducibility and reliability in the sample sets owing to selection bias and differences in etiology, age, sex, mode of onset, treatment regimens and clinical course.

End-stage heart failure is associated with an increased activity and alterations of multiple gene products including the extracellular matrix/cytoskeletal (e.g. collagen types I and III, fibromodulin, fibronectin, and connexin 43 (Tan et al., 2002). When gene expression profile was applied in a transgenic model of tumor necrosis factor-α overexpression, a large number of immune response-related genes, along with a IgG deposition in myocardium, supports activation of immune system and inflammatory mechanism in the development and progression of heart failure (Feldman and McTiernan, 2004; Kubota et al., 1997).

Gene expression profiles of heart failure caused by alcoholic cardiomyopathy and familial cardiomyopathy suggest that the onset and disease progression may involve different genetic determinants. Genomic profiling in a murine model of heart failure reverted to the normal phenotype after rescue by expression β-adrenergic receptor kinase, and suggested mild and advanced heart failure maybe similar in mice and humans (Blaxall et al., 2003).

iii. A Case for Biologic Reclassification of Heart Failure

Gene expression profiling has significantly improved the diagnostic classification of specific conditions (e.g., breast cancer, chronic myelogenous leukemia) but remains a formidable challenge for deciphering meaningful insights about the biological mechanisms underlying disease pathogenesis (Quackenbush, 2006). Among inheritable forms of cardiovascular diseases, recent advances of single-gene disorders have fundamentally altered understanding about the cellular processes, metabolic alterations and transcriptional reprogramming of the diseased heart (Seidman and Seidman, 2001). Much like the success seen for tumor classification and other improvements in cancer therapeutics (Bell, 2004; Quackenbush, 2006), and beyond the availability of genetic tests for disease-causing mutations of cardiomyopathy (Morita et al., 2005), the development of genomic tools that are causally linked to disease pathogenesis, termed a ‘molecular signature,’ can accelerate progress for early detection, targeted therapy and disease monitoring of inheritable heart failure (Bell, 2004). As disclosed herein, opportunities exist for microarray-based profiling, proteomics, metabolomics and genome wide technologies to propel the transition from clinico-pathologic to clinico-genomic classifications for heart failure.

Different gene profiles for failing and nonfailing hearts have already permitted differentiation among heart failure with different etiologies as shown recently Donahue and colleagues in Table 3 (Donahue et al., 2006). Considerable discordance, however, exists between the ability to diagnose heart failure using genomic profile lags substantially behind clinical management. Important obstacles remain in procuring tissue samples needed genomic profiles and their transition from use as research tools into the realm of clinical diagnostics.

TABLE 3
Discovery Projects

| Comparison | Subjects | Platform | Findings |
|---|---|---|---|
| Failing versus | 2 cases (1 ICM | Affymetrix | Alterations of expression of |
| non-failing | and 1 DCM) | Hu 6800 | cytoskeletal and myofibrillar genes, |
| | 2 control cases | | genes encoding stress proteins, and |
| | | | genes involved in metabolism, protein |
| | | | synthesis, and protein degradation. |
| Failing versus | 7 cases (DCM) | Cardiochip | Up-regulation of genes for atrial |
| non-failing | 5 control cases | (custom | natriuretic peptide, sarcomeric and |
| | | array) | cytoskeletal proteins, stress proteins, |
| | | | and transcription/translation |
| | | | regulators. |
| | | | Down-regulation of genes regulating |
| | | | calcium signaling pathways |
| Failing versus | 8 cases (DCM) | Affymetrix | 103 differentially expressed genes |
| non-failing | 7 control cases | Hu 6800 | with most prominent being atrial |
| | | | natriuretic factor and brain natriuretic |
| | | | peptide |
| Failing versus | 10 cases (DCM) | Custom | 364 differentially expressed genes |
| non-failing | 4 control cases | arrays | Up-regulation being most prominent |
| | | | in genes for energy pathways, muscle |
| | | | contraction, electron transport, and |
| | | | intracellular signaling. |
| | | | Down-regulation was most prominent |
| | | | in genes for cell cycle control. |
| Failing versus | 9 cases (5 ICM | Affymetrix | 95 differentially expressed genes with |
| non-failing | and 4 DCM) | HG-U95A | notable up-regulation of atrial |
| | 1 control case | | natriuretic peptide and brain |
| | | | natriuretic peptide. |
| | | | Prominent pathways up-regulated |
| | | | include cell signaling and muscle |
| | | | contraction |
| Failing versus | 6 cases (DCM) | Affymetrix | 165 differentially expressed genes, the |
| non-failing | 5 control cases | HG-U133A | most prominent being structural and |
| | | | metabolic genes |
| Failing versus | 5 cases (DCM) | Custom array | Differentially expressed genes in |
| non-failing | 5 control cases | for apoptotic | apoptotic pathways |
| | | pathways | |
| Pre- and post- | 6 cases (3 DCM | Affymetrix | 530 differentially expressed genes |
| lefT | and 3 ICM) | Hu 6800 | (295 up and ventricular assist device |
| | | | 235 down) with prominent changes in |
| | | | genes for metabolism |
| Pre- and post- | 7 cases (DCM) | Affymetrix | 179 differentially expressed genes |
| left ventricular | | HG-U133A | (130 up and 49 down) |
| assist device | | | There was prominent up-regulation in |
| | | | nitric oxide pathways and down- |
| | | | regulation of inflammatory genes |
| Pre- and post- | 19 cases (8 | Affymetrix | 107 differentially regulated genes (85 |
| left ventricular | DCM and 11 | HG-U133A | up and 22 down) |
| assist devicE | ICM) | | Prominent was the up-regulation of |
| | | | genes regulating vascular networks |
| | | | and down-regulation of genes |
| | | | regulating myocyte hypertrophy. |
| HCM and | 3 DCM | Cardiochip | Multiple genes and pathways up- and |
| DCM versus | 2 HCM | (custom | down-regulated some common to |
| non-failing | 3 control cases | array) | DCM and HCM some distinct to each |

DCM = dilated cardiomyopathy;
HCM = hypertrophic cardiomyopathy;
ICM = ischemic cardiomyopathy.

iv. Biomarkers Vs. Biosignatures for Heart Failure

Considerable biological heterogeneity of heart failure demands more robust tools to guide clinical outcomes. Much recent attention has focused on biological marker, or biomarker, which objectively measures and evaluates normal biological processes, pathologic process, or pharmacological response to therapeutic intervention (Vasan, 2006). Current enthusiasm for biomarker strategies, however, has also brought confusion and ambiguity for applications in clinical practice. Too often, highly fragmented information obtained from patients at different clinical stages precludes meaningful analysis and extrapolation to broader subclasses.

Accordingly, disclosed is an integrative approach that encompasses the ability to predict the onset, rate of progression, and response to therapy and/or clinical outcome with reproducibility and reliability can circumvent such limitation of biomarkers. This approach requires the development of a molecular signature or ‘biosignature.’ Among eight individuals with idiopathic dilated cardiomyopathy but with similar clinical characteristics for chronic heart failure at baseline, serial sampling was superior to cross-sectional gene expression profiling since there was less variance in the differences on gene chip analysis of endomyocardial biopsies from the same patient than among the different subjects with similar phenotypes (Lowes et al., 2006). Because these biological processes can precede the transition into heart failure and premature death, distinct metabolic pathways can be linked to novel molecular signatures in disease pathogenesis (FIG. 23).

As disclosed herein, protein aggregation cardiomyopathy (PAC) (also termed desmin-related myopathy—DRM) is a multi-system disease, caused by the missense R120G mutation in the gene encoding the human small HSP αB-crystallin (hR120GCryAB). Further, selective hR120GCryAB expression in the heart induces a novel toxic gain-of-function mechanism involving reductive stress, apparently emanating from increased activity of glucose 6-phosphate dehydrogenase (G6PD). Reductive stress refers to an abnormal increase in the amounts of reducing equivalents (e.g., glutathione, NADPH), which has been demonstrated in lower eukaryotes (Simons et al., 1995; Trotter and Grant, 2002) but has not been commonly shown in the mammals and/or in disease states (Chance et al., 1979; Gores et al., 1989).

Such genetic evidence, that dysregulation of G6PD activity is a causal mechanism for R120GCryAB cardiomyopathy, forms the rationale for ideas related to metabolic and genetic pathways that might codify biosignatures. What metabolic changes occur before the onset of detectable myopathic or pathologic alterations, and how does such imbalance contribute to cardiomyopathy and heart failure? Does reductive stress exert direct or indirect consequences on mitochondrial (dys)function? Applications in redox proteomics and multiplex protein markers are presently being pursued to determine if glutathionylation, for example, of key components in mitochondrial and other metabolic pathways are causally linked to disease pathogenesis.

v. Molecular Diagnosis of Allograft Rejection

Although peripheral blood mononuclear cells (PBMCs) are abundant and highly accessible sources of genomic material, a potential for diagnostic inaccuracy and therapeutic failure exists if there is discordance between the information in PBMCs and underlying condition in the diseased tissues. Significant progress has been made for patients after cardiac transplantation, which could change existing paradigms for clinical decision-making and management of allograft rejection. Standard protocols after heart transplantation requires patients to undergo serial endomyocardial biopsies (EMB) as a means to monitor for rejection and to guide immunosuppressive therapy. Such surveillance maneuvers are invasive, expensive and carry considerable risks such as perforation of the ventricular wall and hemopericardium. Analysis of the histological data by expert pathologists is subject to inter-observer variability and the diagnosis of acute rejection has been controversial (Nielsen et al., 1993; Winters and McManus, 1996). Gene expression profiles of PBMCs can provide an alternative approach of the diagnosis of allograft rejection (Horwitz et al., 2004). Patients who subsequently developed acute rejection had a distinct genomic profile compared with patients without any rejection and, after treatment for rejection, the majority (98%) of differentially expressed genes returned to baseline.

The CARGO (Cardiac Allograft Rejection Gene Expression Observational) study prospectively investigated gene expression analysis from PMBCs as a diagnostic tool to predict transplant rejection (Mehra, 2005). From the core group of 11 genes associated with immune response pathways, which were identified by quantitative real-time polymerase chain reaction (QT-PCR) and assigned weighted scores, CARGO investigators were able to predict rejection with a sensitivity and specificity of 80% and 60%, respectively (Deng et al., 2006). Owing to reduced sensitivity and specificity immediately after transplantation, the test can also be unreliable for the diagnosis of low/intermediate grade rejection. Now commercially available (AlloMap®), this landmark study provides proof-of-concept that gene expression profiling in peripheral blood monocyte cells (PBMCs), which were predictive for acute rejection pathways in cardiac transplant patients.

vi. Method

Provided herein is a method of predicting, detecting, or monitoring a condition caused or exacerbated by reductive stress in a subject, comprising measuring concentrations of one or more nucleic acids or proteins involved in glutathione metabolism in a tissue or bodily fluid of the subject, wherein a measurable increase in one or more of the nucleic acids or proteins is an indication of the condition in the subject. Thus, also provided is a method of predicting, detecting, or monitoring cardiomyopathy or risk of developing cardiomyopathy in a subject, comprising measuring concentrations of one or more nucleic acids or proteins involved in glutathione metabolism in a tissue or bodily fluid of the subject, wherein a measurable increase in one or more of the nucleic acids or proteins is an indication of cardiomyopathy in the subject.

In some aspects, the nucleic acids or proteins involved in glutathione metabolism is Glucose-6-phosphate dehydrogenase (G6PD). In some aspects, the nucleic acids or proteins involved in glutathione metabolism is glutathione peroxidase 1 (Gpx1). Gpx1 can have the sequence set forth in Genbank Accession No. BG065030. In some aspects, the nucleic acids or proteins involved in glutathione metabolism is glutathione peroxidase 3 (Gpx3). Gpx3 can have the sequence set forth in Genbank Accession No. BG073718. In some aspects, the nucleic acids or proteins involved in glutathione metabolism is glutathione S-transferase, alpha 4 (Gsta4). Gsta4 can have the sequence set forth in Genbank Accession No. BG073190. In some aspects, the nucleic acids or proteins involved in glutathione metabolism is glutathione S-transferase, mu 1 (Gstm1). Gstm1 can have the sequence set forth in Genbank Accession No. BG086970 or BG074397. In some aspects, the nucleic acids or proteins involved in glutathione metabolism is microsomal glutathione S-transferase 1 (Mgst1). Mgst1 can have the sequence set forth in Genbank Accession No. BG086330.

In some aspects, the method comprises measuring 1, 2, 3, 4, 5, or 6 of G6PD, Gpx1, Gpx3, Gsta4, Gstm1, or Mgst1. Thus, the method can comprise measuring G6PD, Gpx1, Gpx3, Gsta4, Gstm1, and Mgst.

Thus, the method can comprise measuring G6PD and Gpx1. Thus, the method can comprise measuring G6PD and Gpx3. Thus, the method can comprise measuring G6PD and Gsta4. Thus, the method can comprise measuring G6PD and Gstm1. Thus, the method can comprise measuring G6PD and Mgst. Thus, the method can comprise measuring Gpx1 and Gpx3. Thus, the method can comprise measuring Gpx1 and Gsta4. Thus, the method can comprise measuring Gpx1 and Gstm1. Thus, the method can comprise measuring Gpx1 and Mgst. Thus, the method can comprise measuring Gpx3 and Gsta4. Thus, the method can comprise measuring Gpx3 and Gstm1. Thus, the method can comprise measuring Gpx3, Gsta4 and Mgst. Thus, the method can comprise measuring Gsta4 amd Gstm1. Thus, the method can comprise measuring Gsta4 and Mgst. Thus, the method can comprise measuring Gstm1 and Mgst.

Thus, the method can comprise measuring G6PD, Gpx1, Gpx3, Gsta4, and Gstm1. Thus, the method can comprise measuring G6PD, Gpx1, Gpx3, Gsta4, and Mgst. Thus, the method can comprise measuring G6PD, Gpx1, Gpx3, Gstm1, and Mgst. Thus, the method can comprise measuring G6PD, Gpx1, Gsta4, Gstm1, and Mgst. Thus, the method can comprise measuring G6PD, Gpx3, Gsta4, Gstm1, and Mgst. Thus, the method can comprise measuring Gpx1, Gpx3, Gsta4, Gstm1, and Mgst.

In some aspects, detection of an increase of at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, or 400% for each of the nucleic acids or proteins measured that are involved in glutathione metabolism indicates that the subject has or is at risk of developing a condition caused or exacerbated by reductive stress, such as cardiomyopathy.

In some aspects, detection of an increase of at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, or 400% for 1, 2, 3, 4, 5, or 6 of the nucleic acids or proteins measured that are involved in glutathione metabolism indicates that the subject has or is at risk of developing a condition caused or exacerbated by reductive stress, such as cardiomyopathy.

### 4. Diagnosing and Monitoring Reductive Stress Conditions

Also provided is a method of predicting, detecting, or monitoring reductive stress in a subject, comprising measuring concentrations of reductants and oxidants, such as reduced and oxidized glutathione, homocysteine (and other thiols) in a tissue or bodily fluid of the subject.

Thus, the herein disclosed methods can comprise the detection of reductants and oxidantsin bodily fluid of the subject, such as blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

Blood plasma is the liquid component of blood, in which the blood cells are suspended. Plasma is the largest single component of blood, making up about 55% of total blood volume. Serum refers to blood plasma in which clotting factors (such as fibrin) have been removed. Blood plasma contains many vital proteins including fibrinogen, globulins and human serum albumin. Sometimes blood plasma can contain viral impurities which must be extracted through viral processing.

Standard methods for detecting and distinguishing oxidants and reductants in a tissue sample or bodily fluid can be used and include HPLC.

### 5. Screening Method

Also provided herein is a method of identifying an agent that can be used to treat a condition caused or exacerbated by reductive stress. The method can comprise screening chemical libraries of small molecules. The biological pathways in cultured cells can be genetically engineered to exhibit reductive stress. Cultured cells can be monitored for signs of reductive stress. For example, the cells can be monitored using reduction-oxidation green fluorescent protein (roGFPs). Initial ‘hits’ of small molecules contained in chemical library can serve as chemical probes to conduct large-scale screening capacity. Chemical probes can be tested in both cultured cells and small animal models of human diseases to reverse or prevent protein aggregation, cardiac hypertrophy, and pathologic features of reductive stress. Such molecules (existing or synthetic) are likely to treat diseases caused by reductive stress.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the activity on reductive stress should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits a condition caused or exacerbated by reductive stress. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions, such as those disclosed herein.

Candidate agents encompass numerous chemical classes, but are most often organic molecules, e.g., small organic compounds having a molecular weight of more than 100 and less than about 2,500 daltons. For example, the molecules can have an average molecular weight 500 daltons or less. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

### 6. Methods of Administration

A composition disclosed herein may be administered in a number of ways depending on whether local or systemic treatment is desired, and on the area to be treated. For example, the compositions may be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, a typical daily dosage of an anti-reductant used alone might range from about 1 μg/kg to up to 100 mg/kg of body weight or more per day, depending on the factors mentioned above.

For example, the thiuram disulfide compound disulfuram can be effective when administered at an amount within the conventional clinical ranges determined in the art. Typically, it can be effective in a human subject at an amount of from about 125 to about 1000 mg per day, such as from about 250 to about 500 mg per day. However, the amount can vary with the body weight of the patient treated. The active ingredient may be administered at once, or may be divided into a number of smaller doses to be administered at predetermined intervals of time. The suitable dosage unit for each administration of disulfuram can be, e.g., from about 50 to about 1000 mg, such as from about 250 to about 500 mg. The desirable peak plasma concentration of disulfuram generally is about 0.05 to about 10 μM, preferably about 0.5 to about 5 μM, in order to achieve a detectable therapeutic effect. However, a plasma concentration beyond such ranges may work as well.

Disulfuram has been used clinically in treating alcohol abuse. A dosage form of disulfuram approved by the U.S. Food and Drug Administration (Antabuseo®) can be purchased in 250 and 500 mg tablets for oral administration from Wyeth-Ayerst Laboratories (P.O. Box 8299, Philadelphia, Pa. 19101, Telephone 610-688-4400).

Disulfuram implanted subcutaneously for sustained release has also been shown to be effective at an amount of 800 to 1600 mg to achieve a suitable plasma concentration. This can be accomplished by using aseptic techniques to surgically implant disulfuram into the subcutaneous space of the anterior abdominal wall. See, e.g., Wilson et al., J. Clin. Psych. 45:242-247 (1984).

A sustained release dosage formulation comprised to 80% poly(glycolic-co-L-lactic acid) and 20% disulfuram has also been described in Phillips et al., J. Pharmaceut. Sci. 73:1718-1720 (1984).

The pharmacology and toxicology of Antabuse® are detailed in Physicians Desk Reference, 50th edition, Medical Economics, Montvale, N.J., pages 2695-2696. Steady-state serum levels of approximately 1.3 μM have been measured in humans taking repeated doses of 250 mg disulfuram daily. See, e.g., Faiman et al., Clin. Pharmacol. Ther. 36:520-526 (1984); and Johansson, Acta Psychiatr. Scand., Suppl. 369:15-26 (1992). Disulfuram is relatively non-toxic, with an LD₅₀ in rodents of 8.6 g/kg. See, e.g., The Merck Index, 10th Edition, Reference 3382, Merck & Co., Rahway, N.J., 1983, page 491.

Disulfuram can be used in a similar dosage in the disclosed methods. The therapeutically effective amount for other thiuram disulfide compounds may also be estimated or calculated based on the above dosage ranges of disulfuram and the molecular weights of disulfuram and the other thiuram disulfide compounds, or by other methods known in the art.

Heavy metal ions can be administered separately as an aqueous solution in a pharmaceutically suitable salt form. However, they can also be administered in a chelate form in which the ions are complexed with thiuram disulfide compounds. Thus, the amount of heavy metal ions to be used advantageously is proportional to the amount of thiuram disulfide compound to be administered based on the molar ratio between a heavy metal ion and thiuram disulfide compound in the chelate. Methods for preparing such chelates or complexes are known and the preferred methods are disclosed above and in the examples below.

Following administration of a disclosed composition for treating, inhibiting, or preventing a condition caused or exacerbated by reductive stress, the efficacy of the therapeutic can be assessed in various ways well known to the skilled practitioner. For instance, one of ordinary skill in the art will understand that a composition disclosed herein is efficacious in treating or inhibiting a condition caused or exacerbated by reductive stress in a subject by observing that the composition restores homeostasis, for example by measureing the level of reductants, such as reduced glutathione (GSH) and comparing it to the level of oxidants, such as oxidized glutathione (GSSG). Reductants can be measured by methods that are known in the art, for example, using HPLC to detect the presence of the reduced and oxidized protein in a sample (e.g., but not limited to, blood) from a subject or patient.

The compositions that inhibit reductive stress disclosed herein may be administered prophylactically to patients or subjects who are at risk for reductive stress or who have been newly diagnosed with a condition caused or exacerbated by reductive stress.

The disclosed compositions and methods can also be used for example as tools to isolate and test new drug candidates for a variety of reductive-stress related diseases.

### 7. Methods of Making

The compositions disclosed herein and the compositions necessary to perform the disclosed methods can be made using any method known to those of skill in the art for that particular reagent or compound unless otherwise specifically noted.

i. Transgenic Models

Provided herein is a method of making the herein disclosed non-human animal model of protein aggregation cardiomyopathy, comprising administering to a non-human mammal a nucleic acid encoding human αB-crystallin (CryAB) protein, wherein the protein comprises a mutation at residue 120.

a. Methods of Producing Transgenic Animals

The nucleic acids and vectors provided herein can be used to produce transgenic animals. Various methods are known for producing a transgenic animal. In one method, an embryo at the pronuclear stage (a “one cell embryo”) is harvested from a female and the transgene is microinjected into the embryo, in which case the transgene will be chromosomally integrated into the germ cells and somatic cells of the resulting mature animal. In another method, embryonic stem cells are isolated and the transgene is incorporated into the stem cells by electroporation, plasmid transfection or microinjection; the stem cells are then reintroduced into the embryo, where they colonize and contribute to the germ line. Methods for microinjection of polynucleotides into mammalian species are described, for example, in U.S. Pat. No. 4,873,191, which is incorporated herein by reference. In yet another method, embryonic cells are infected with a retrovirus containing the transgene, whereby the germ cells of the embryo have the transgene chromosomally integrated therein. When the animals to be made transgenic are avian, microinjection into the pronucleus of the fertilized egg is problematic because avian fertilized ova generally go through cell division for the first twenty hours in the oviduct and, therefore, the pronucleus is inaccessible. Thus, the retrovirus infection method is preferred for making transgenic avian species (see U.S. Pat. No. 5,162,215, which is incorporated herein by reference). If microinjection is to be used with avian species, however, the embryo can be obtained from a sacrificed hen approximately 2.5 hours after the laying of the previous laid egg, the transgene is microinjected into the cytoplasm of the germinal disc and the embryo is cultured in a host shell until maturity (Love et al., Biotechnology 12, 1994). When the animals to be made transgenic are bovine or porcine, microinjection can be hampered by the opacity of the ova, thereby making the nuclei difficult to identify by traditional differential interference-contrast microscopy. To overcome this problem, the ova first can be centrifuged to segregate the pronuclei for better visualization.

The transgene can be introduced into embryonal target cells at various developmental stages, and different methods are selected depending on the stage of development of the embryonal target cell. The zygote is the best target for microinjection. The use of zygotes as a target for gene transfer has a major advantage in that the injected DNA can incorporate into the host gene before the first cleavage (Brinster et al., Proc. Natl. Acad. Sci., USA 82:4438-4442, 1985). As a consequence, all cells of the transgenic non-human animal carry the incorporated transgene, thus contributing to efficient transmission of the transgene to offspring of the founder, since 50% of the germ cells will harbor the transgene.

A transgenic animal can be produced by crossbreeding two chimeric animals, each of which includes exogenous genetic material within cells used in reproduction. Twenty-five percent of the resulting offspring will be transgenic animals that are homozygous for the exogenous genetic material, 50% of the resulting animals will be heterozygous, and the remaining 25% will lack the exogenous genetic material and have a wild type phenotype.

In the microinjection method, the transgene is digested and purified free from any vector DNA, for example, by gel electrophoresis. The transgene can include an operatively associated promoter, which interacts with cellular proteins involved in transcription, and provides for constitutive expression, tissue specific expression, developmental stage specific expression, or the like. Such promoters include those from cytomegalovirus (CMV), Moloney leukemia virus (MLV), and herpes virus, as well as those from the genes encoding metallothionein, skeletal actin, phosphenolpyruvate carboxylase (PEPCK), phosphoglycerate (PGK), dihydrofolate reductase (DHFR), and thymidine kinase (TK). Promoters from viral long terminal repeats (LTRs) such as Rous sarcoma virus LTR also can be employed. When the animals to be made transgenic are avian, preferred promoters include those for the chicken [bgr]-globin gene, chicken lysozyme gene, and avian leukosis virus. Constructs useful in plasmid transfection of embryonic stem cells will employ additional regulatory elements, including, for example, enhancer elements to stimulate transcription, splice acceptors, termination and polyadenylation signals, ribosome binding sites to permit translation, and the like.

In the retroviral infection method, the developing non-human embryo can be cultured iin vitro to the blastocyst stage. During this time, the blastomeres can be targets for retroviral infection (Jaenich, Proc. Natl. Acad. Sci. USA 73:1260-1264, 1976). Efficient infection of the blastomeres is obtained by enzymatic treatment to remove the zona pellucida (Hogan et al., Manipulating the Mouse Embryo (Cold Spring Harbor Laboratory Press, 1986). The viral vector system used to introduce the transgene is typically a replication-defective retrovirus carrying the transgene (Jahner et al., Proc. Natl. Acad. Sci., USA 82:6927-6931, 1985; Van der Putten et al., Proc. Natl. Acad. Sci. USA 82:6148-6152, 1985). Transfection is easily and efficiently obtained by culturing the blastomeres on a monolayer of virus producing cells (Van der Putten et al., supra, 1985; Stewart et al., EMBO J. 6:383-388, 1987). Alternatively, infection can be performed at a later stage. Virus or virus-producing cells can be injected into the blastocoele (Jahner et al., Nature 298:623-628, 1982). Most of the founders will be mosaic for the transgene since incorporation occurs only in a subset of the cells which formed the transgenic nonhuman animal. Further, the founder can contain various retroviral insertions of the transgene at different positions in the genome, which generally will segregate in the offspring. In addition, it is also possible to introduce transgenes into the germ line, albeit with low efficiency, by intrauterine retroviral infection of the mid-gestation embryo (Jahner et al., supra, 1982).

Embryonal stem cell (ES) also can be targeted for introduction of the transgene. ES cells are obtained from pre-implantation embryos cultured iin vitro and fused with embryos (Evans et al. Nature 292:154-156, 1981; Bradley et al., Nature 309:255-258, 1984; Gossler et al., Proc. Natl. Acad. Sci., USA 83:9065-9069, 1986; Robertson et al., Nature 322:445-448, 1986). Transgenes can be efficiently introduced into the ES cells by DNA transfection or by retrovirus mediated transduction. Such transformed ES cells can thereafter be combined with blastocysts from a nonhuman animal. The ES cells thereafter colonize the embryo and contribute to the germ line of the resulting chimeric animal (see Jaenisch, Science 240:1468-1474, 1988).

“Founder” generally refers to a first transgenic animal, which has been obtained from any of a variety of methods, e.g., pronuclei injection. An “inbred animal line” is intended to refer to animals which are genetically identical at all endogenous loci.

b. Crosses

It is understood that the animals provided herein can be crossed with other animals. For example, wherein the provided animals are mice, they can be crossed with Alzheimer's Mice to study the effects of inflammatory mediators, e.g. IL-1β, on Alzheimer's disease. The association between Aβ deposition and inflammatory changes is reinforced by studies of transgenic mice harboring familial AD mutant genes. In transgenic mice expressing the Swedish APP mutation (Tg2576, APP_{K670N,M671L}; hereafter referred to as APPsw), microglial activation is intimately related to amyloid plaque deposition, with measures of both microglial size and activated microglial density being highest in the immediate vicinity of Aβ deposits [Frautschy, S. A, et al. Am. J. Pathol. (1998) 152:307-317]. These mice accumulate Aβ deposits over a protracted period of time, with plaques and glial changes becoming prominent after one year of age [Hsiao, K., P. Chapman, S, Nilsen, C. Eckman, Y. Harigaya, S. Younkin, F. Yang and G. Cole. Science (1996) 274:99-102]. Although other AD mouse models are available, the APPsw mice have been extensively characterized and offer an excellent resource for investigating mechanisms involved in Aβ deposition or Aβ induced inflammatory changes.

Other dystrophic transgenic animals can be crossed with the provided transgenic animals. Many mutant animal models of muscular dystrophy share common genetic and protein abnormalities similar to those of the human disease. The best example is a model of Duchenne muscular dystrophy (DMD), the mdx mouse (Collins et al. Int J Exp Pathol. 2003 84(4):165-72; De Luca et al. Neuromuscul Disord. 2002 12 Suppl 1:S142-6). Similar to dystrophic muscle in DMD patients, dystrophin protein is not expressed along the surface membrane, even though the mdx mouse has no apparent signs of muscular dysfunction. Because clinical and pathologic findings in the dystrophic (mxd) dog are similar to those in DMD patients, it also has been regarded as a good model for therapeutic trials. The best known and most extensively studied dy+dy+ mouse lacks merosin (laminin alpha2), which is one subunit of a basement membrane protein, laminin. Because approximately half of all patients with the classical form of congenital muscular dystrophy also lack merosin, availability of this animal has revived interest in the study of the pathologic mechanism of fiber necrosis resulting from this membrane defect. The dystrophic hamster is a model of limb-girdle muscular dystrophy with sarcoglycan deficiency in which one of the dystrophin-associated glycoproteins, delta-sarcoglycan, is defective. Because these animal models have common protein and genetic defects similar to those seen in people with muscular dystrophies, they have been widely used to examine the effectiveness of gene therapy and the administration of pharmacologic and trophic factors. Other examples of dystrophic animals include those with altered expression of Fukutin (Taniguchi et al. Hum Mol. Genet. 2006 15(8):1279-89) or Nesprin-2 (Zhang et al. J Cell Sci. 2005 118(Pt 4):673-87).

ii. Delivery of the Compositions to Cells

Animal models of protein aggregation cardiomyopathy can also be produce by exogenous delivery of the disclosed mutant CyrAB or nucleic acids encoding the disclosed mutant CyrAB directly to the heart. Thus, also provided herein are compositions and methods for the delivery of a nucleic acid encoding the disclosed mutant CyrAB to a cardiac cell. There are a number of compositions and methods which can be used to deliver nucleic acids to cells, either iin vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a. Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as the nucleic acids encoding an inflammation molecule into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes; they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(A) Retroviral Vectors

A retrovirus is an animal virus belonging to the virus family of Retroviridae, including any types, subfamilies, genus, or tropisms. In Microbiology-1985, American Society for Microbiology, pp. 229-232, Washington, (1985), which is incorporated by reference herein, retroviral vectors, in general, are described by Verma, I. M., Retroviral vectors for gene transfer. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(B) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(C) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(D) Lentiviral Vectors

The vectors can be lentiviral vectors, including but not limited to, SIV vectors, HIV vectors or a hybrid construct of these vectors, including viruses with the HIV backbone. These vectors also include first, second and third generation lentiviruses. Third generation lentiviruses have lentiviral packaging genes split into at least 3 independent plasmids or constructs. Also, vectors can be any viral family that shares the properties of these viruses which make them suitable for use as vectors. Lentiviral vectors are a special type of retroviral vector which are typically characterized by having a long incubation period for infection. Furthermore, lentiviral vectors can infect non-dividing cells. Lentiviral vectors are based on the nucleic acid backbone of a virus from the lentiviral family of viruses. Typically, a lentiviral vector contains the 5′ and 3′ LTR regions of a lentivirus, such as SIV and HIV. Lentiviral vectors also typically contain the Rev Responsive Element (RRE) of a lentivirus, such as SIV and HIV.

(1) Feline Immunodeficiency Viral Vectors

One type of vector that the disclosed constructs can be delivered in is the VSV-G pseudotyped Feline Immunodeficiency Virus system developed by Poeschla et al. Nature Med. (1998) 4:354-357 (Incororated by reference herein at least for material related to FIV vectors and their use). This lentivirus has been shown to efficiently infect dividing, growth arrested as well as post-mitotic cells. Furthermore, due to its lentiviral properties, it allows for incorporation of the transgene into the host's genome, leading to stable gene expression. This is a 3-vector system, whereby each confers distinct instructions: the FIV vector carries the transgene of interest and lentiviral apparatus with mutated packaging and envelope genes. A vesicular stomatitis virus G-glycoprotein vector (VSV-G; Burns et al., Proc. Natl. Acad. Sci. USA 90:8033-8037. 1993) contributes to the formation of the viral envelope in trans. The third vector confers packaging instructions in trans (Poeschla et al. Nature Med. (1998) 4:354-357). FIV production is accomplished iin vitro following co-transfection of the aforementioned vectors into 293-T cells. The FIV-rich supernatant is then collected, filtered and can be used directly or following concentration by centrifugation. Titers routinely range between 10⁴-10⁷ bfu/ml.

(E) Packaging Vectors

As discussed above, retroviral vectors are based on retroviruses which contain a number of different sequence elements that control things as diverse as integration of the virus, replication of the integrated virus, replication of un-integrated virus, cellular invasion, and packaging of the virus into infectious particles. While the vectors in theory could contain all of their necessary elements, as well as an exogenous gene element (if the exogenous gene element is small enough) typically many of the necessary elements are removed. Since all of the packaging and replication components have been removed from the typical retroviral, including lentiviral, vectors which will be used within a subject, the vectors need to be packaged into the initial infectious particle through the use of packaging vectors and packaging cell lines. Typically retroviral vectors have been engineered so that the myriad functions of the retrovirus are separated onto at least two vectors, a packaging vector and a delivery vector. This type of system then requires the presence of all of the vectors providing all of the elements in the same cell before an infectious particle can be produced. The packaging vector typically carries the structural and replication genes derived from the retrovirus, and the delivery vector is the vector that carries the exogenous gene element that is preferably expressed in the target cell. These types of systems can split the packaging functions of the packaging vector into multiple vectors, e.g., third-generation lentivirus systems. Dull, T. et al., “A Third-generation lentivirus vector with a conditional packaging system” J. Virol 72(11):8463-71 (1998)

Retroviruses typically contain an envelope protein (env). The Env protein is in essence the protein which surrounds the nucleic acid cargo. Furthermore cellular infection specificity is based on the particular Env protein associated with a typical retrovirus. In typical packaging vector/delivery vector systems, the Env protein is expressed from a separate vector than for example the protease (pro) or integrase (in) proteins.

(F) Packaging Cell Lines

The vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals. One type of packaging cell line is a 293 cell line.

(G) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently iin vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

b. Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or iin vitro.

Thus, the compositions can comprise, in addition to the disclosed nucleic acids or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

Nucleic acids that are delivered to cells which are to be integrated into the host cell genome typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c. In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

iii. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

iv. Peptide Synthesis

One method of producing the disclosed proteins, such as SEQ ID NO:2, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant GA (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

v. Process Claims for Making the Compositions

Disclosed are processes for making the compositions as well as making the intermediates leading to the compositions. For example, disclosed are nucleic acids in SEQ ID NOs:4, 5, 6, or 7. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NOs:4, 5, 6, or 7 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NOs:4, 5, 6, or 7, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth SEQ ID NOs:4, 5, 6, or 7 and a sequence controlling the expression of the nucleic acid.

Disclosed are nucleic acid molecules produced by the process comprising linking in an operative way a nucleic acid molecule comprising a sequence encoding a peptide set forth in SEQ ID NO:3 and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth in SEQ ID NO:3 and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in SEQ ID NO:3, wherein any change from SEQ ID NO:3 is conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### C. KITS

The materials described above as well as other materials can be packaged together in any suitable combination as a kit useful for performing, or aiding in the performance of, the disclosed method. It is useful if the kit components in a given kit are designed and adapted for use together in the disclosed method.

### D. USES

Disclosed are compositions and methods for diagnosing, treating, and/or preventing conditions involving reductive stress. The disclosed compositions can also be used in a variety of ways as research tools. Other uses are disclosed, apparent from the disclosure, and/or will be understood by those in the art.

### E. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a polypeptide” includes a plurality of such polypeptides, reference to “the polypeptide” is a reference to one or more polypeptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### F. EXAMPLES

### 1. Example 1

### Dysregulation of Glutathione Homeostasis Triggers Pathogenic Shifts of Oxido-Reductive Stress and Cardiomyopathy in R120GCryAB Mice

i. Nonstandard Abbreviations

GSH, reduced glutathione; GSSG, oxidized glutathione; hR120GCryAB, human R120G αB-crystallin; G6PD, glucose-6-phosphate dehydrogenase; PAM, protein aggregation myopathy; DRM, desmin related myopathy; m120G, mouse R120G; α-MHC, alpha-myosin heavy chain; Ntg, non-transgenic; ANF, atrial natriuretic factor; BNF, brain natriuretic factor; SSA, sulfosalicilic acid; PSSG, protein disulfides; γ-GCS, γ-glutamyl cysteine synthetase; GSH-R, glutathione reductase; GPx, glutathione peroxidase; MDA, malondialdehyde; DNPH, di-nitrophenyl hydrazine; Aid, protein phosphatase B; ERK, extracellular response kinase; RNS, reactive nitrogen species; hR120G High, human R120G High; hR120G Low, human R120G Low; hsf1, heat shock factor 1; TBST, tris-buffered saline-tween 20; TBARS, thio barbituric acid-reactive substances; PLN, phospholamban, SERCA-2A, sarcoplasmic endoplasmic reticulum calcium ATPase 2A.

ii. Results

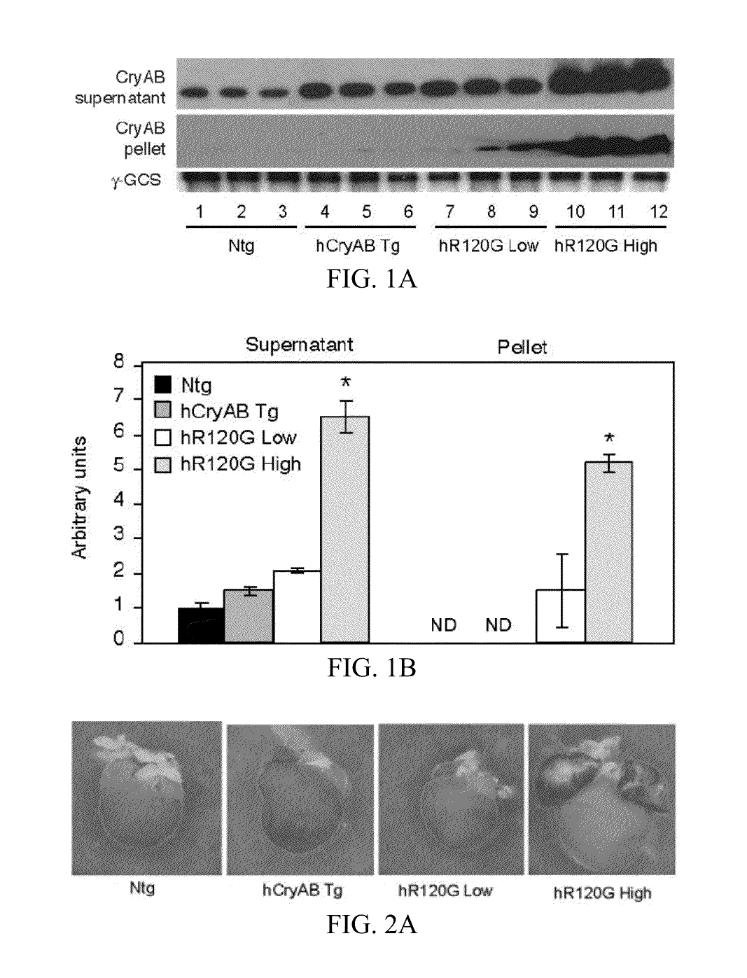

Transgene overexpression in human WT and R120GCryAB in mice: To recapitulate a small animal model of human R120GCryAB expression (hR120G), transgenic mice were generated using the mouse α-myosin heavy chain (α-MHC) promoter driving either the human cDNA CryAB wild type (hCryAB Tg) sequence or R120G mutated form in a tissue-specific manner. Two transgenic lines were established for each construct; lines 3241 and 3244 for α-MHC hCryAB Tg and lines 7302 and 7313 for α-MHC hR120GCryAB. Transgene transmission to the off-spring was analyzed by Southern blot and PCR, which followed the expected Mendelian ratios. CryAB protein in both supernatant and pellet fractions was probed by Western blot for each experimental group representing nontransgenic (NTg), hCryAB Tg and hR120GCryAB genotypes (FIG. 1). Total levels of CryAB protein expression were 1.5 fold greater in hCryAB Tg compared with NTg (FIG. 1, NTg lanes 1-3 vs. hCryAB Tg lanes 4-6). Total CryAB protein, reflecting endogenous and transgene expression, was 2-fold in line 7313 hR120G and 6-fold in line 7302 hR120G greater than NTg (FIG. 1A and FIG. 1B). These transgenic lines, with mild and moderate CryAB overexpression, were, therefore, designated hR120G Low and hR120G High, respectively. In both lines 3241 and 3244, the wild type human transgene (hCryAB Tg) protein expression was similar to hR120G Low (FIG. 1A and FIG. 1B). Whereas hCryAB Tg remained entirely soluble, hR120GCryAB was found in both soluble and insoluble fractions, indicating that mutant protein expression recapitulates the protein ‘surplus’ disorder, a proposed model for desmin-related myopathies (Vicart, P., et al. 1998; Wang, X., et al. 2001: Wang, X., et al. 2001).

Pathologic Features and Decreased Survival in hR120G High Cardiomyopathic Mice:

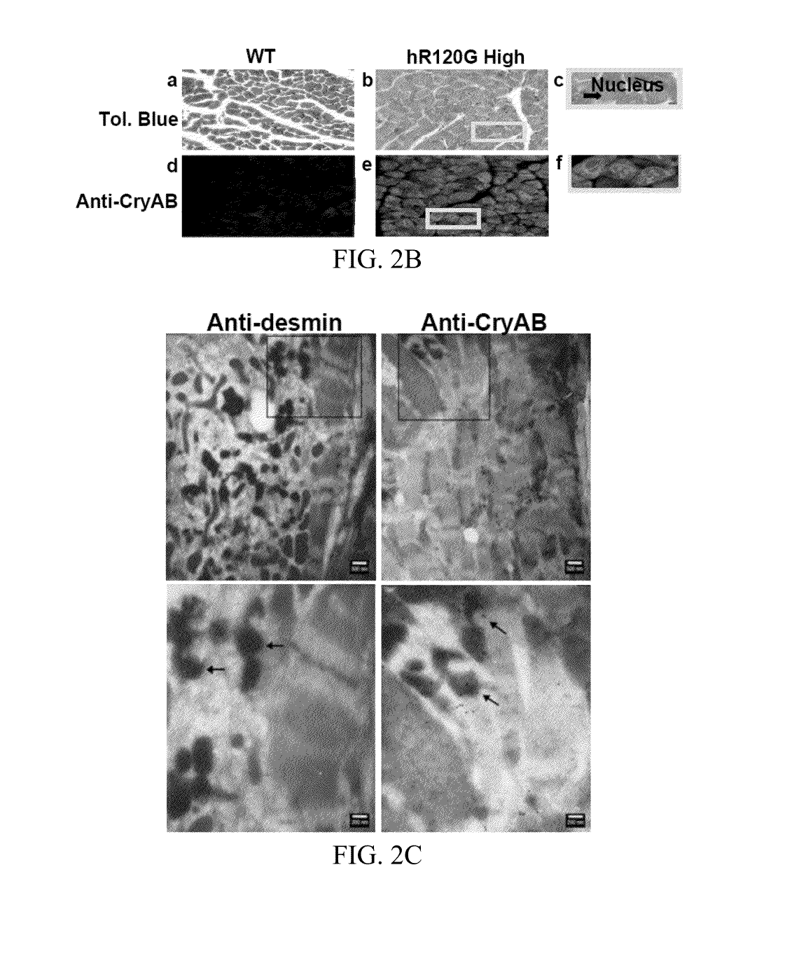

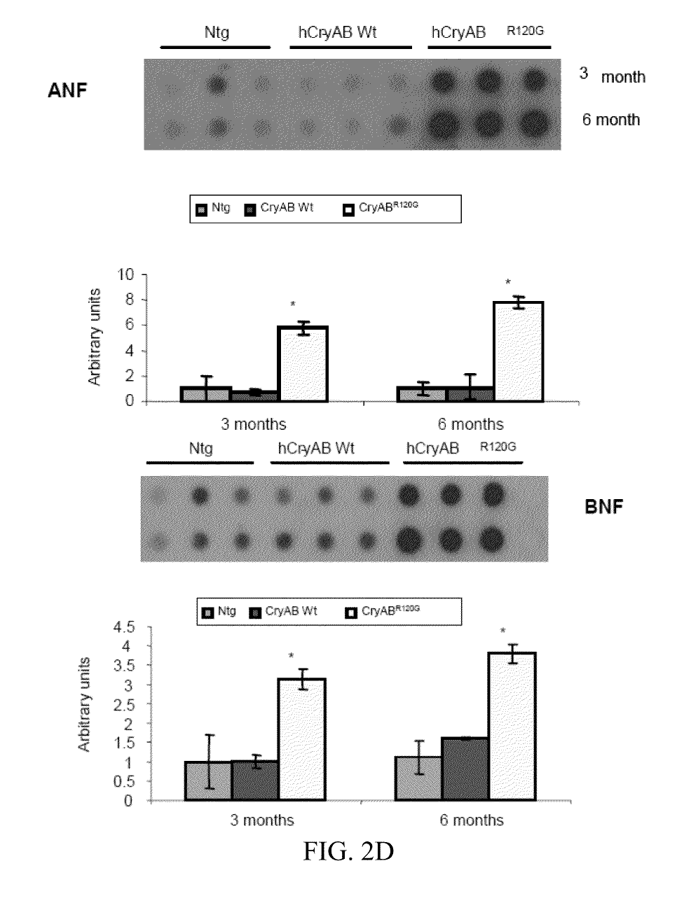

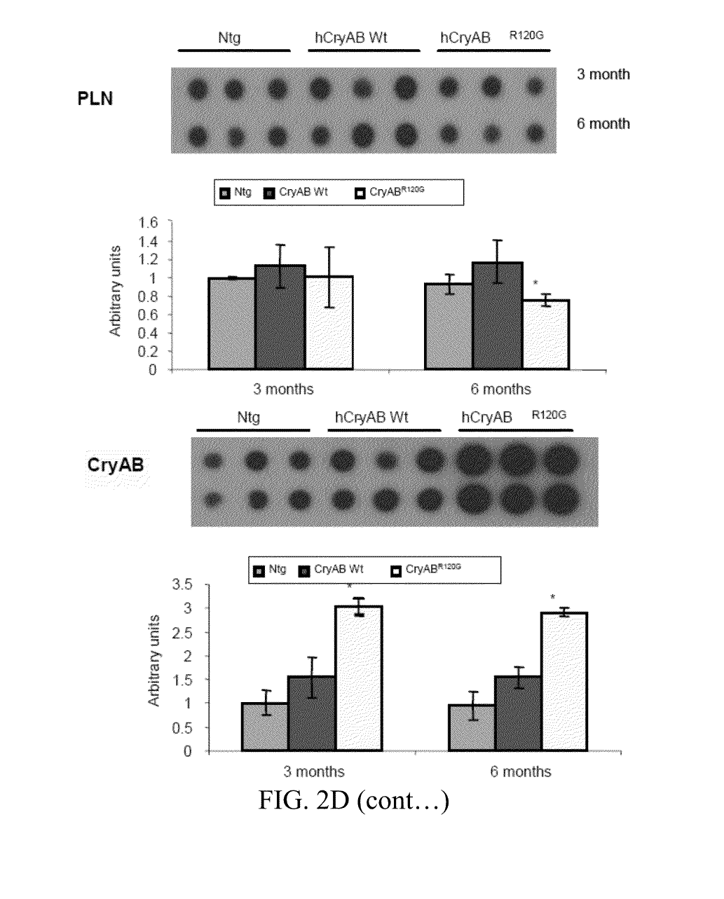

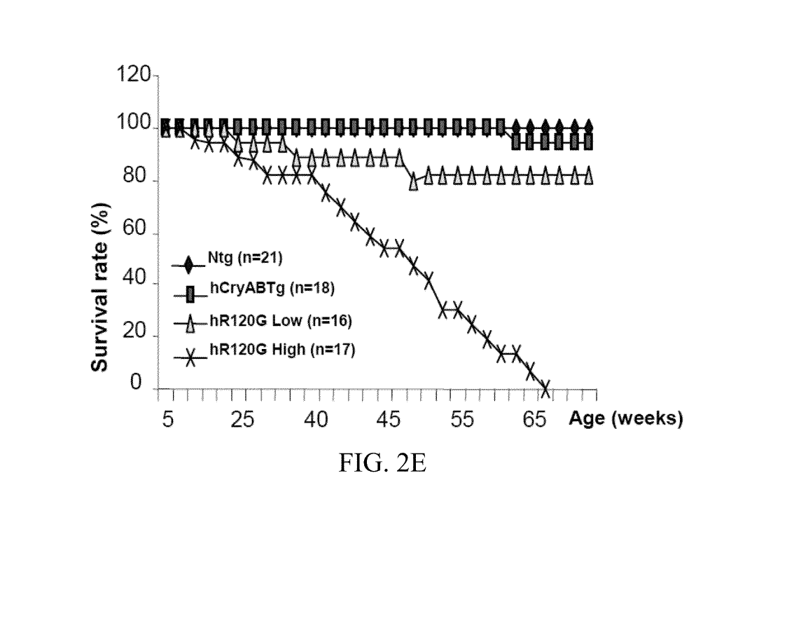

Moderate overexpression of hR120G in mouse heart induced cardiac hypertrophy as shown by heart weight/body weight ratio (Table 4). Consistently, morphological analyses revealed gross four-chamber enlargement, biatrial thrombosis and cardiac hypertrophy, especially in hR120GCryAB High (FIG. 2A). On routine histological sections, hypertrophy of cardiac myocytes containing large aggregates was present in essentially all hR120GCryAB High hearts at 6 months. FIG. 2B shows perinuclear aggregates in toluidine-blue stained myocardial sections (FIG. 2B, panels a-c) and immunohistochemical stained sections with anti-CryAB (FIG. 2B, panels d-f).

DRM has been characterized at the ultrastructural level by the presence of electron dense aggregates at Z-lines structures, which are remarkably indistinguishable for either desmin or CryAB disease-causing mutations (Fardeau, M., et al. 1978). Consistent with earlier reports (Goebel, H. H., et al. 2000), large aggregates containing dense granulomatous materials seen only in hR120G High hearts were positive for immunogold particles against either CryAB or desmin at the ultrastructural level (FIG. 2C). Control hearts were devoid of aggregates.

At the molecular level, Northern blot showed that markers of congestive heart failure, such as atrial natriuretic factor (ANF), brain natriuretic factor (BNF), and CryAB were all increased at 3- and 6-months, whereas phospholamban expression, a major regulator of cardiac contractility and relaxation, was decreased with the onset of heart failure in 6-month old hR120G High myopathic hearts (FIG. 2D).

While morpho-pathologic signs were clearly detected at 6 months, the rate of disease progression accelerated more severely afterwards for hR120G High reaching 100% mortality by 14 months, hR120G Low exhibited 20% mortality after 20 months (FIG. 2E), and no detrimental effects on the mortality of either hCryAB Tg mice or nontransgenic (NTg) littermates were observed over 20 months (FIG. 2E).

MRI Studies of Human CryAB Cardiomyopathy in Mice:

To non-invasively assess the effects of hR120GCryAB expression on cardiac function, magnetic imaging resonance (MRI) was used and serial measurements of ventricular cavity dimension, left ventricular mass (LVM) and left ventricular ejection fraction (LVEF) were obtained at 3, 6 and 10 months (Table 5). The hCryAB Tg mouse line, with mild wild-type CryAB overexpression, which exhibit normal growth and postnatal development, was selected as a control. In both 3- and 6-month animals, no differences in cavity dimension and cardiac function were observed among the hCryAB Tg, hR120G Low and hR120G High. There was a trend for greater LVM for hR120GCryAB High compared with either hR120GCryAB Low or hCryAB Tg at 6 months (103.6±4.77 vs 88.1±2.81 mg or 93.2±2.17, respectively). Cardiac hypertrophy was most pronounced in hR120G High at 10 months when compared with hR120GCryAB Low and/or hCryAB Tg (LVM 129.8±6.93 vs 103.6±4.77 vs 88.3±3.98 mg, p<0.0001). Likewise, cardiac dysfunction accompanying decreased ejection fraction was seen at 10 months for hR120G High compared with either hR120GCryAB Low or hCryAB Tg and LVEF 41.3±9.23 vs 57±3.26; p<0.0001 and p<0.2, respectively). Therefore, cardiac hypertrophy and severe ventricular remodeling with dilatation are specific hallmarks of end-stage hR120G protein aggregation cardiomyopathy in mice.

R120G Low Mice Exhibit Decreased Cardiac Contractile Reserve:

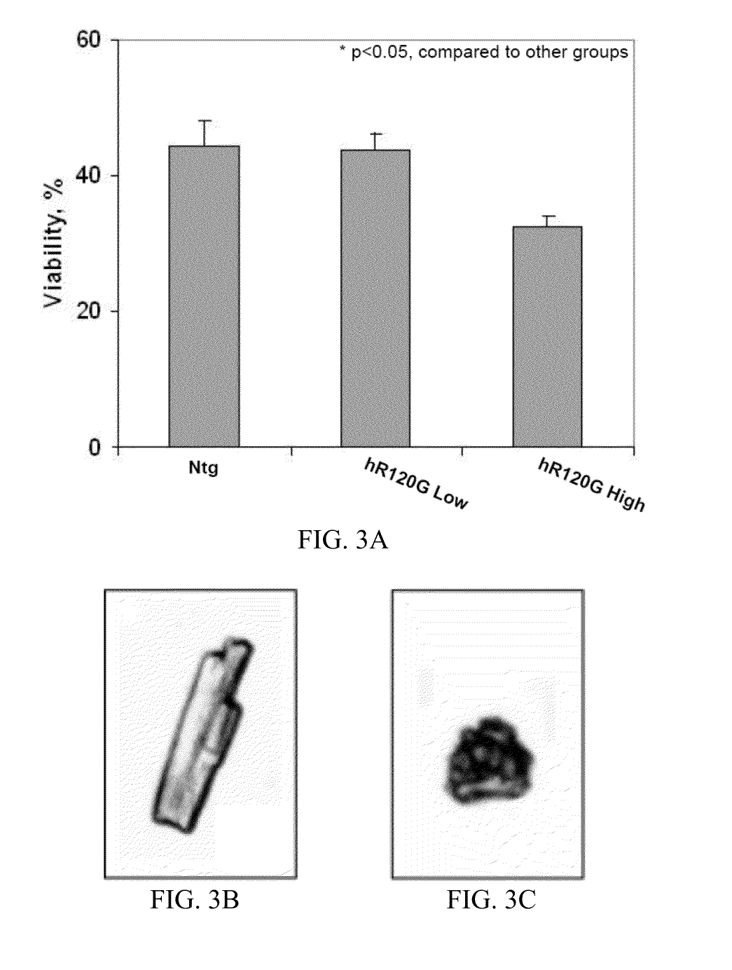

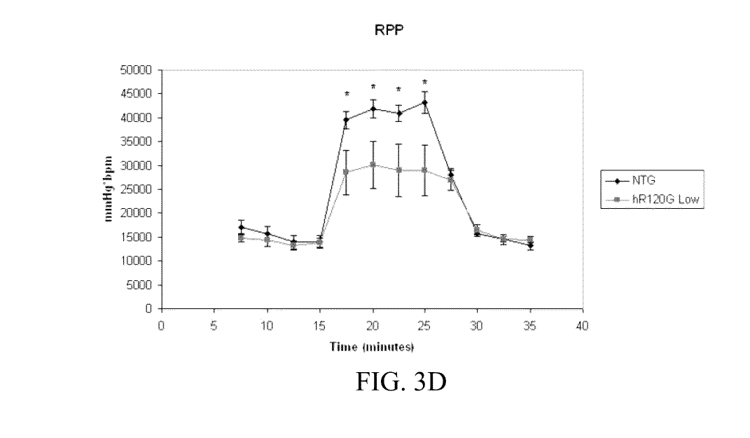

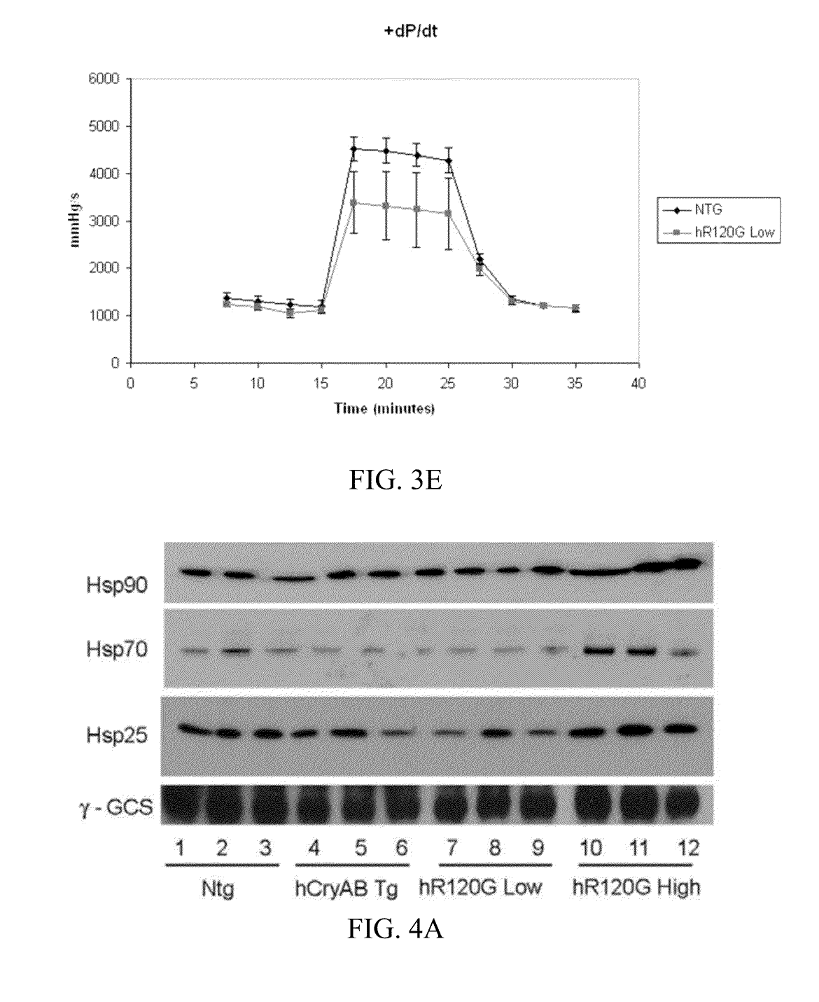

To assess the effects of hR120GCryAB on cardiac myocyte viability, isolated left ventricular myocytes were incubated in culture medium at 30° C. in a 5% CO₂ atmosphere for 1 hour. At least 100 myocytes were observed with phase contrast microscopy (Nikon TMS), and the % percentage with a normal rod shape was taken as an index of viability (Boston, D. R., et al. 1998), the survival of hR120G High cardiomyocytes was reduced 30% compared with either hR120g Low or NTg (FIG. 3A).

Given that myocyte viability of hR120G Low was similar to NTg (FIG. 3A), and the foregoing MRI studies showing that cardiac function (i.e., LVED) of hR120G Low was unchanged between 3 and 10 months under basal conditions (Table 5), it was next investigated whether hR120G Low mice with mild CryAB overexpression exerted any cardiovascular functional consequences. To assess for more subtle abnormalities, experimental groups were subjected to dobutamine challenge, a widely used ex vivo assessment of cardiac reserve. FIG. 3B shows that myocardial external work and maximal rates of contraction before, during, and after exposure to 300 nM dobutamine in the isolated perfused Langendorff heart. While there was a trend for increased maximal rate of contraction (+dP/dt), the derivative of the measured LVDP, external work (RPP), represented as the product of heart rate (HR) and left ventricular developed pressure (LVDP), was significantly decreased in hearts of hR120G Low mice compared with NTg during dobutamine stimulation (FIG. 3B). These findings are consistent with the notion that hR120G Low with mild CryAB overexpression exerts myopathic effects in vivo.

Major Hsps, Especially Hsp25, are Induced by Mutant hCryAB Tg Expression:

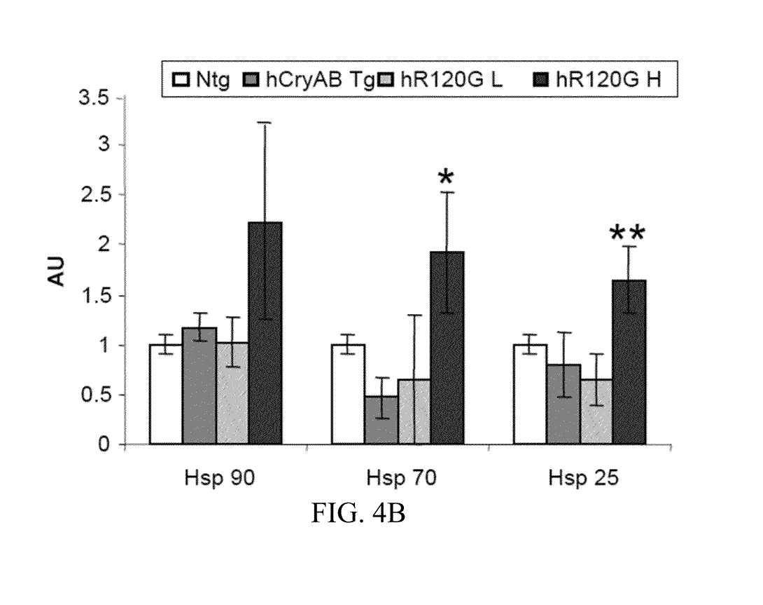

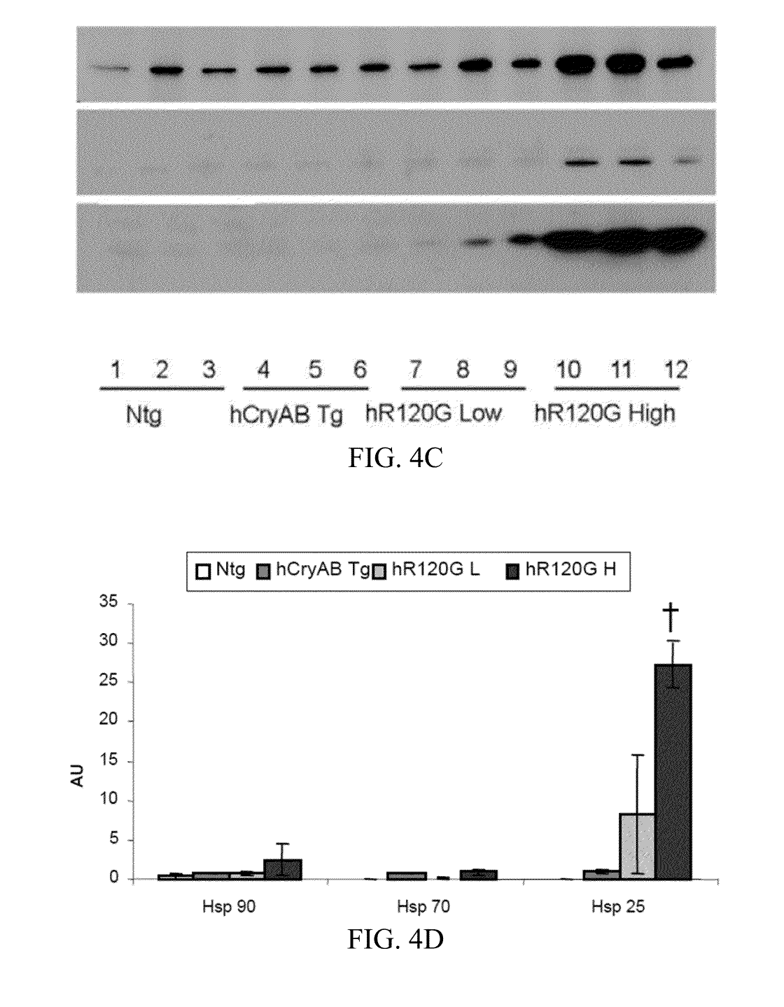

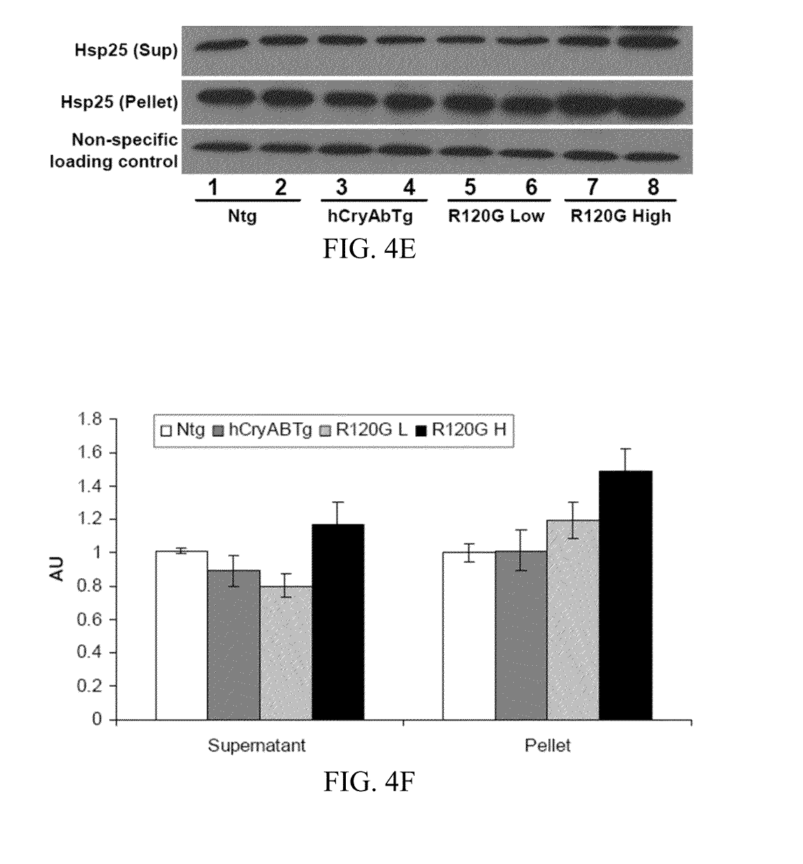

The multigene families of heat shock proteins and regulatory factors constitute an important defense system for limiting aberrant aggregation and for mitigating deleterious sequelae of misfolded protein expression (Christians, E. S., et al. 2002; Williams, R. S., et al. 2000). To characterize the effects of hR120G overexpression on this molecular pathway in myopathic hearts, representative members of the major Hsp families were initially assessed by Western blot analysis in age-matched animals at 6 months, an arbitrary transition point associated with progression of heart failure and increased mortality (FIG. 2E). For all four age-matched experimental groups (n=3 animals/group) at 6 months, FIG. 4 shows the composite Hsp expression panel in which each lane represents a different animal per genotype. Levels of Hsp90, an ATP-dependent chaperone that forms multiprotein complexes, were 2-fold higher for hR120G High than in NTg, hCryAB Tg, or hR120G Low hearts (FIG. 4A-FIG. 4D) in both soluble and insoluble fractions. Likewise, Hsp70 levels were increased by 2-fold in the soluble fraction of cardiac homogenates with hR120GCryAB expression. Whereas Hsp25 protein, a non-ATP dependent chaperone that forms multimeric oligomers, was modestly increased in the supernatant fraction, this chaperone was >25 fold higher in the insoluble fraction of hR120G High than either NTg, hCryAB Tg, or hR120G Low heart homogenates (FIG. 4C, FIG. 4D). Of note, levels of Hsp25 were indistinguishable among these four experimental groups at 2 months (FIG. 4E, FIG. 4F), indicating that progressive expression of hR120G mutant triggers upregulation of stress-inducible Hsps in vivo. As protein abundance of Hsp25 was greater in the insoluble fraction of hR120G High than hR120G Low (FIG. 4D), these findings indicate that increased subcompartmental translocation and/or interaction with the cytoskeleton correlate directly with the dosage of hR120GCryAB expression.

Biomarkers of Oxidative Stress are Altered by hR120G Expression:

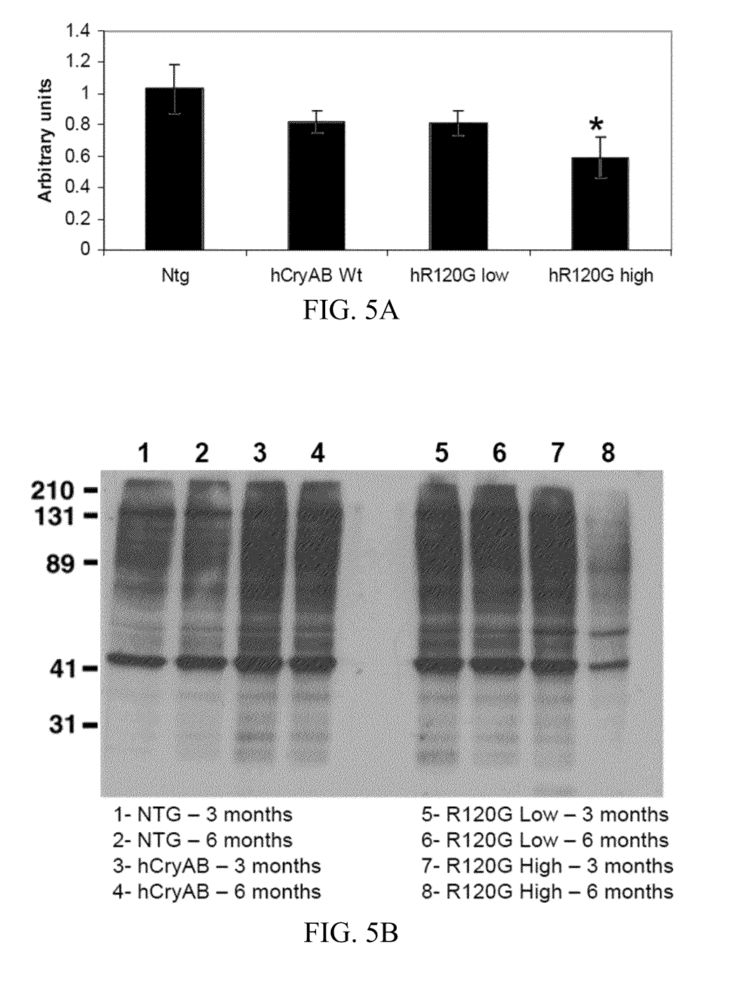

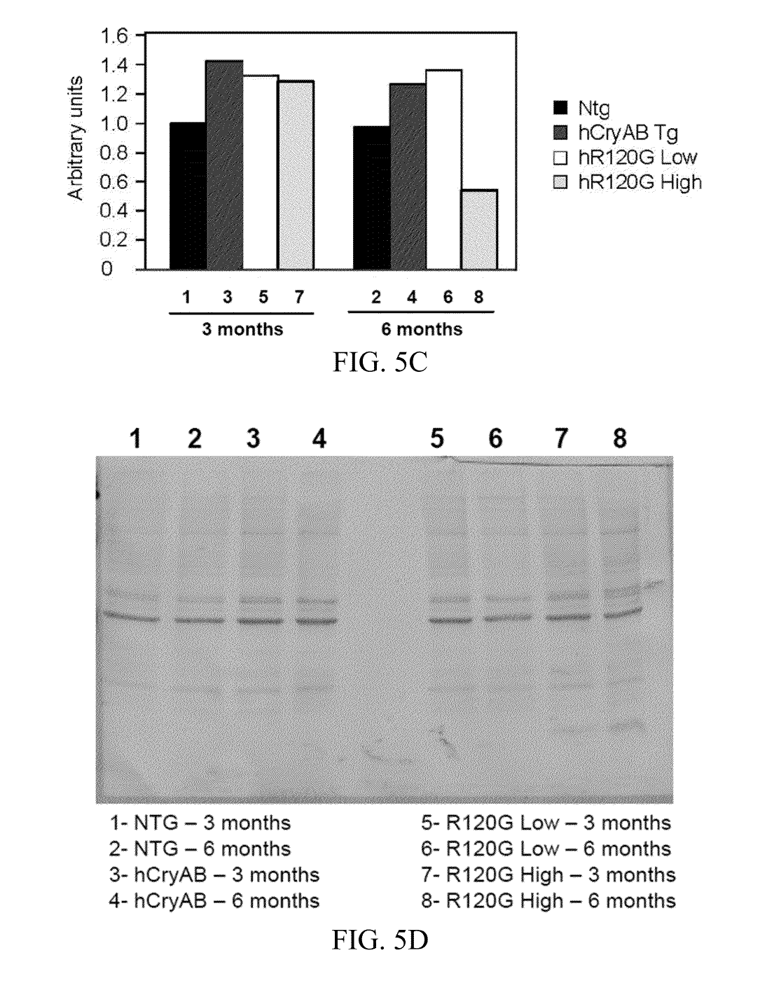

As reactive oxygen species (ROS) have been implicated in the pathogenesis of cardiac hypertrophy and heart failure, the susceptibility of intracellular lipids and proteins to undergo oxidative modifications as surrogate biomarkers was next assessed (Yamamoto, M., et al. 2003; Giordano, F. J. 2005; Griendling, K. K., et al. 2003). FIG. 5A shows that measurements of lipid peroxidation using malondialdehyde (MDA), a biomarker of oxidative stress, were significantly and unexpectedly lower (by 40%) in hR120G High at 6 months compared with Ntg control (1.03±0.16 and 0.59±0.13; Ntg vs. hR120G High, p<0.05) (FIG. 5A). To corroborate these age-dependent effects, myocardial levels of protein carbonyl content were assessed by anti-DNPH immunostaining for specific amino acid residues modified by reactive oxygen species (Stadtman, E. R. 1992). At 3 and 6 months, tissue levels of anti-DNP immunoreactive proteins were also elevated in both hCryAB Tg and hR120G Low compared with Ntg (FIG. 5B, FIG. 5C). In contrast, there was profound lowering of protein carbonyl levels in hR120G High between 3 and 6 months (FIG. 5B, FIG. 5C, compare lanes 7 and 8). Thus, conditions leading to a reversal of biomarkers for oxidative stress can be linked to the pathogenic mechanism(s) of hR120G High cardiomyopathic mice.

hR120G Expression Causes Oxido-Redox Shift Towards Reductive Stress:

The reversal in the carbonyl content in hR120G High at 6 months is consistent with either an exaggerated increase in the antioxidant mechanisms or marked enhancement in the reducing equivalents, or both. To test these hypotheses of the effects of hR120G expression, it was first asked whether myopathic hearts respond with increased GSH level and alterations in redox balance. Table 6 shows the concentrations of reduced (GSH), oxidized glutathione (GSSG) and protein bound thiols (PSSG) in 6-month old experimental groups. In heart homogenates, the relative amounts of GSH revealed the following rank order: hR120G High >hR120G Low >hCryAB Tg >Non-Tg. Indeed, the total GSH content of hR120G High is significantly increased by ˜2-fold compared with NTg (1573.02+33.57 vs 811.19+125.87, p<0.05). The amount of GSSG in the different Tg groups was 25% higher than Ntg, each displaying equivalent GSSG amounts at 6 months (Table 6). However, the higher GSH:GSSG ratio hR120G High did not reach statistical significance compared with hR120G Low, hCryAB Tg, or Non-Tg at 6 months.

As shifts in the abundance of reduced GSH might react with intracellular proteins to form protein disulfides (PSSG), the PSSG content in the heart homogenates was examined Myocardial levels of PSSG were lowest in the hR120G High homogenates at 6 months. Taken together, these results indicate that the effects of high level of hR120GCryAB dramatically increases the reducing power, exemplified by the GSH concentrations and higher GSH:GSSG ratio (i.e., >50 arbitrary units).

hR120G High Expression Activates the GSH Biosynthesis Recycling Pathway:

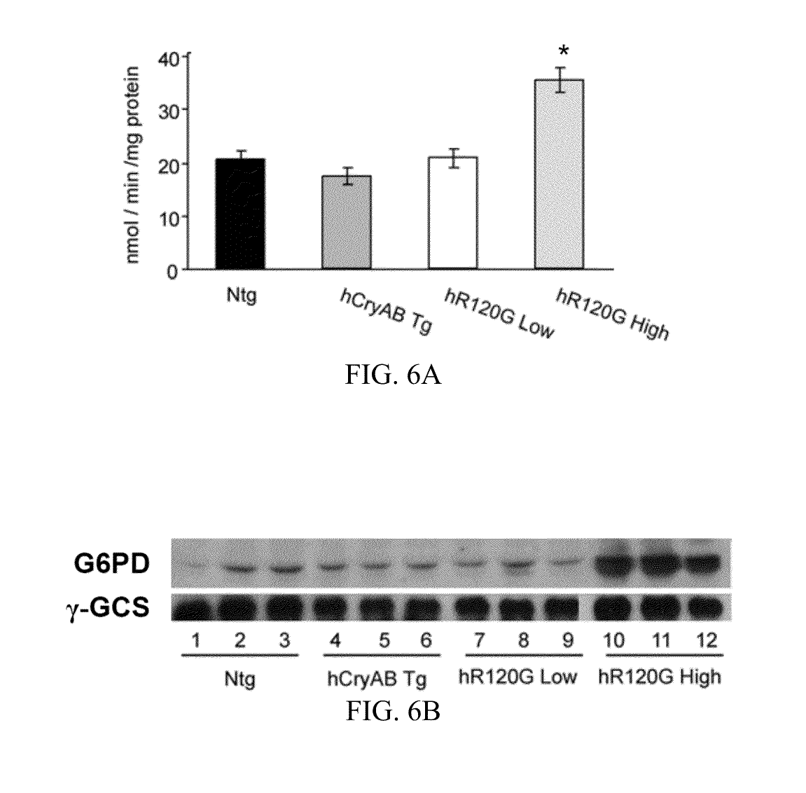

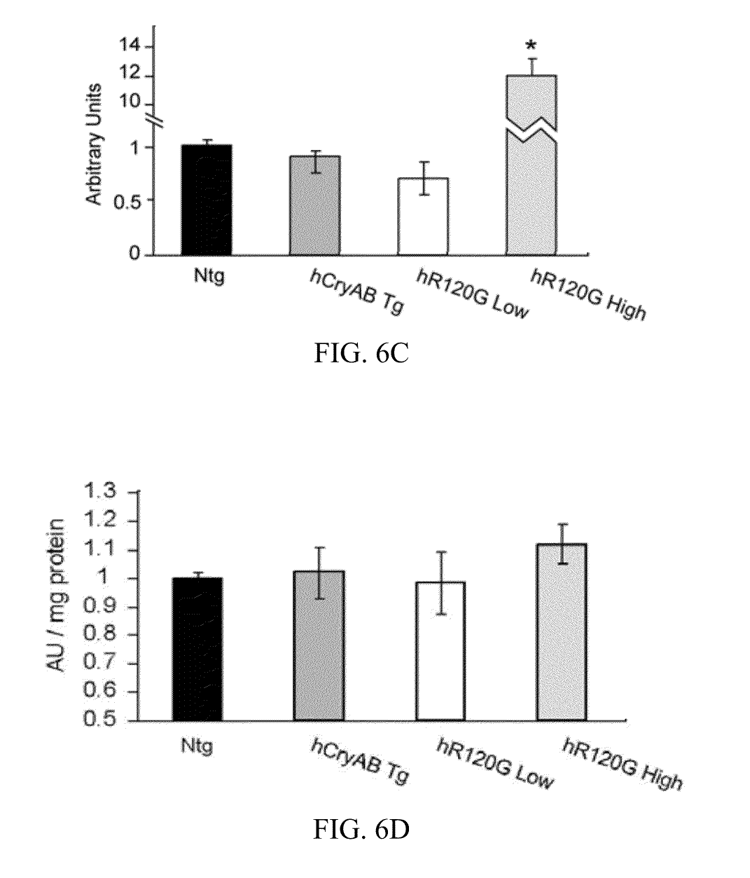

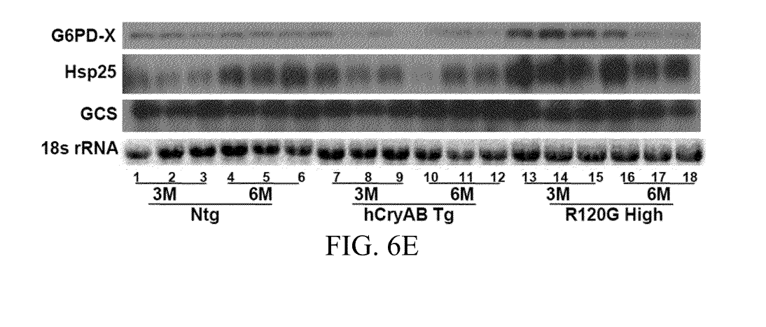

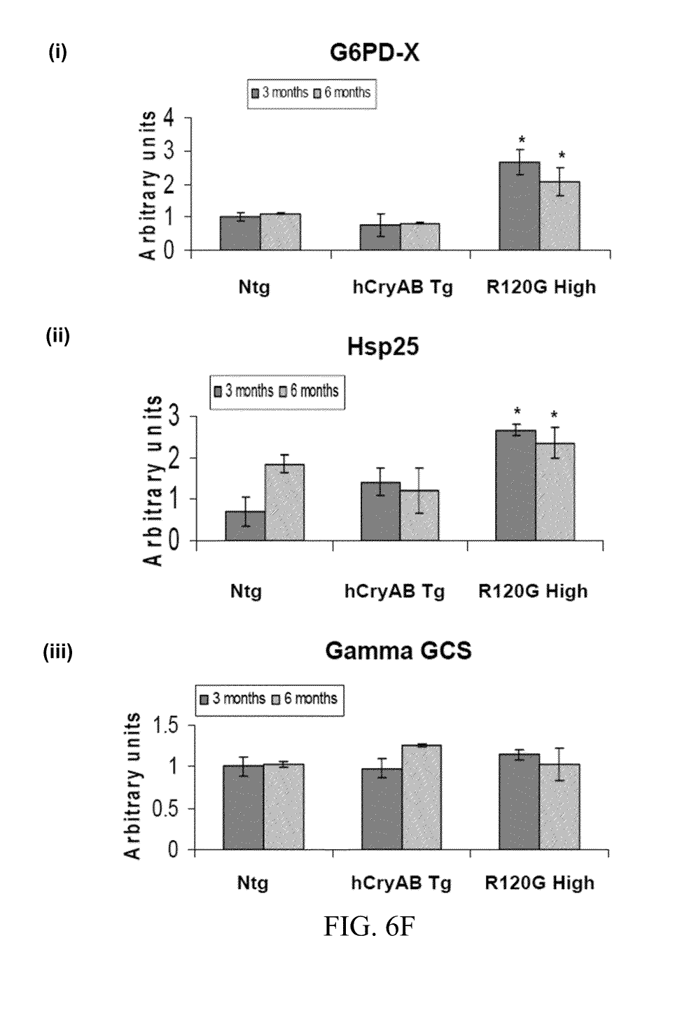

Insights about the mechanism(s) for GSH overproduction in hR120G High cardiomyocytes warranted a systematic assessment of each enzymatic step that catalyzes either the recycling and/or de novo synthesis pathways. Reduced GSH is produced either from oxidized GSSG by the oxidation of the co-factor nicotinamide adenine-dinucleotide phosphate, NADPH, a product of glucose-6-phosphate dehydrogenase (G6PD), the rate-limiting enzyme of the pentose phosphate pathway (Preville, X., et al. 1999). Myocardial abundance of G6PD protein, however, was 12-fold higher in hR120G High than NTg, hCryAB Tg, or hR120G Low at 24 weeks or 6 months (FIG. 6B, FIG. 6C). The G6PD enzyme activity in heart homogenates of hR120G High was 2-fold greater than NTg, hCryAB Tg, or hR120G Low at 6 months (FIG. 6A). In parallel to increase in G6PD enzyme activity, mRNA levels of G6PD were higher by 2.5 fold and 2.0 fold at 3- and 6-month old hR120G High than either Ntg or hCryAB Tg, respectively. Thus, transcriptional mechanisms involving oxido-reductive pathways, in part, underlie the pathogenic shift of hR120G High with moderate mutant CryAB overexpression in vivo (FIG. 6E). These findings are noteworthy since additional mechanisms, besides upregulation of G6PD transcription, can account for 12-fold increase in protein abundance of G6PD in hR120G High mice.

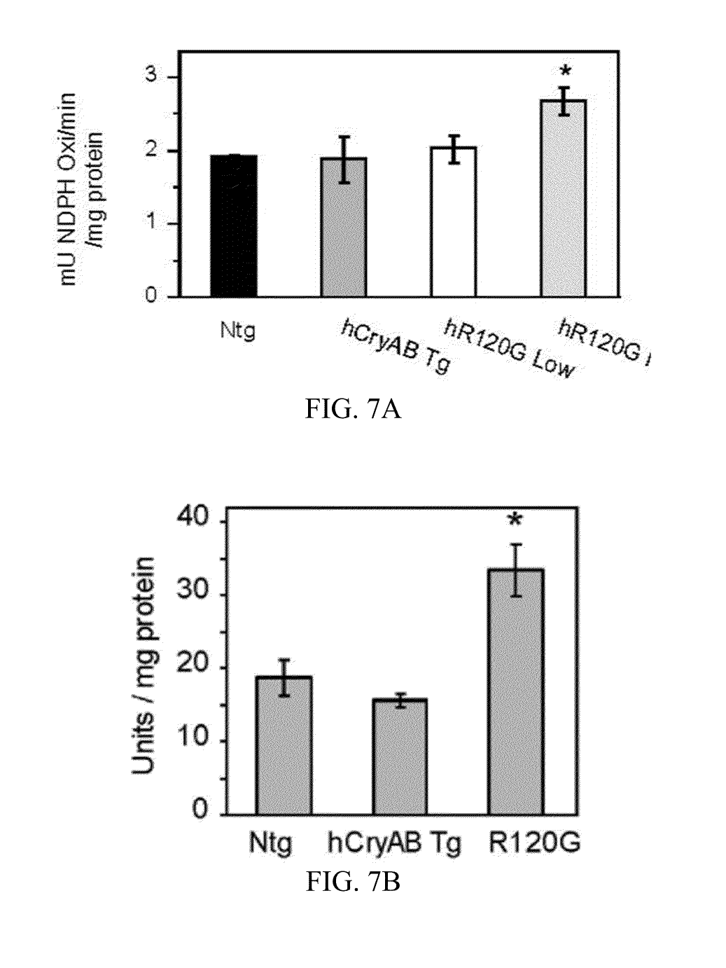

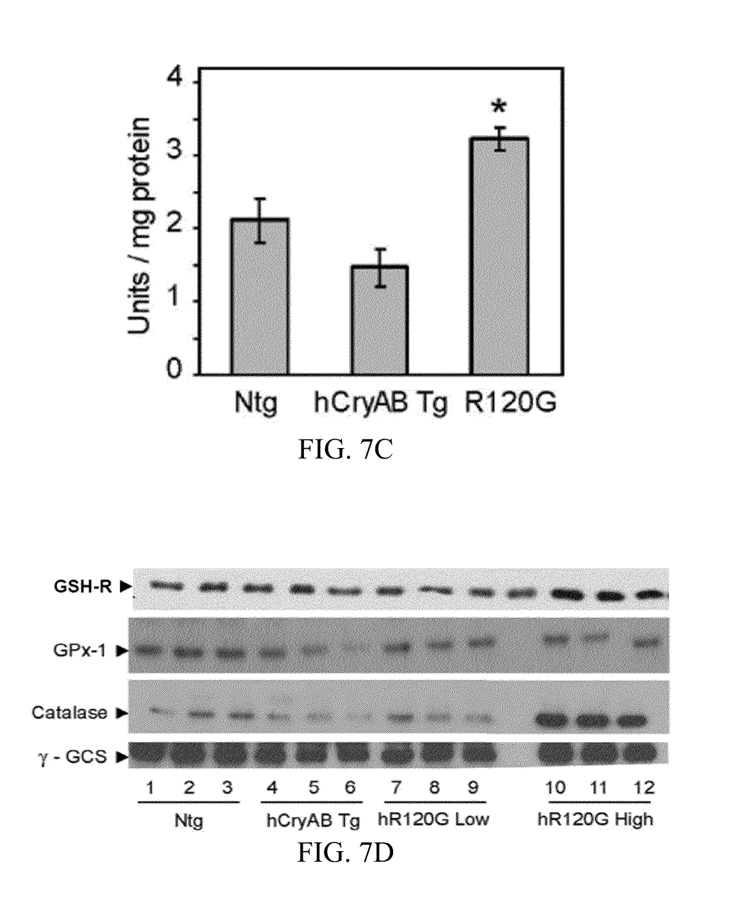

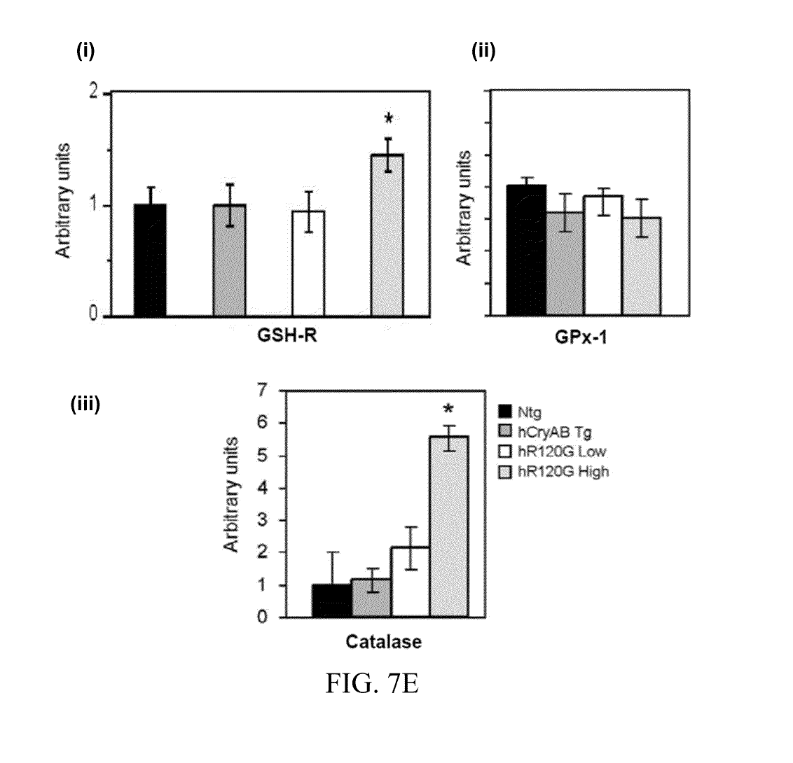

Next, glutathione reductase (GSH-R) activity, which uses NADPH as the principal source of reducing equivalents for recycling oxidized GSSG to reduced GSH, was tested. Whereas both enzymatic activity and protein content of GSH-R were similar in NTg, hCryAB Tg, and hR120G Low at 6 months, the corresponding values for GSH-R were also significantly increased by hR120G High expression in age-matched animals (FIG. 7A, FIG. 7D & FIG. 7E). Indeed, enzymatic activity and protein abundance of gamma-glutamyl cysteine synthetase (γ-GCS), the rate-limiting enzyme for biosynthesis under feedback inhibition by GSH, were indistinguishable among all experimental groups examined (FIG. 6B, FIG. 6D). Therefore, the GSH recycling pathway, and not de novo biosynthesis, was the predominant mechanism for elevated GSH levels in response to dose-dependent hR120GCryAB expression.

Antioxidative Mechanisms are Increased Before Decompensation of hR120G High Mice:

It was next examined whether temporal changes in the redox imbalance and higher reductive stress might be accompanied by the induction of antioxidant pathways associated with increased demands for detoxifying increased reactive oxygen species in vivo. Previous studies have demonstrated that chronic exposure to hydrogen peroxide induces glutathione peroxidase and catalase (Carper, D., et al. 2001), and that forced overexpression of antioxidant enzymes affords effective cytoprotective defense mechanisms (Hollander, J. M., et al. 2003; Ye, G., et al. 2004). The enzymatic activity of glutathione peroxidase (GPx), which catalyzes the elimination of peroxides, was 70% higher in hR120G High compared with Ntg (FIG. 7B). While cytosolic glutathione peroxidase assessed by immunoblot analysis was similar among all groups (FIG. 7D, FIG. 7E (panel (ii))), the enzymatic activity of catalase in hR120G High was 50% and 100% higher than either NTg or hCryAB Tg, respectively (FIG. 7C). Furthermore, protein abundance of catalase in hR120G High was 2- and 5-fold higher than either NTg or hCryAB Tg (p<0.05) (FIG. 7D, FIG. 7E (panel (iii))). Taken together, these findings support the notion that hR120G High expression enhances antioxidative enzymatic pathways in response, in part, to elevated oxidative stress and preceding pathogenic reductive stress.

hR120G High Expression Promotes Protein-Protein Interactions with GSH Biosynthetic Machinery:

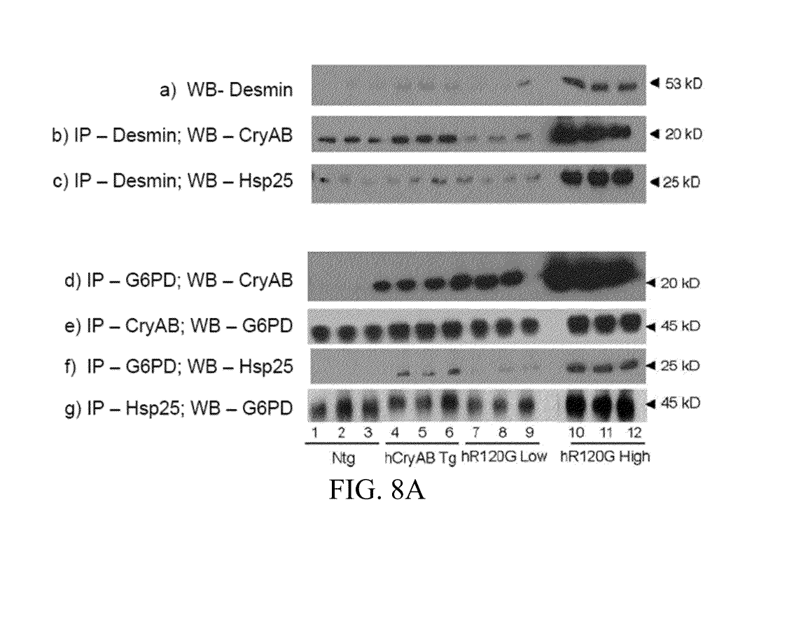

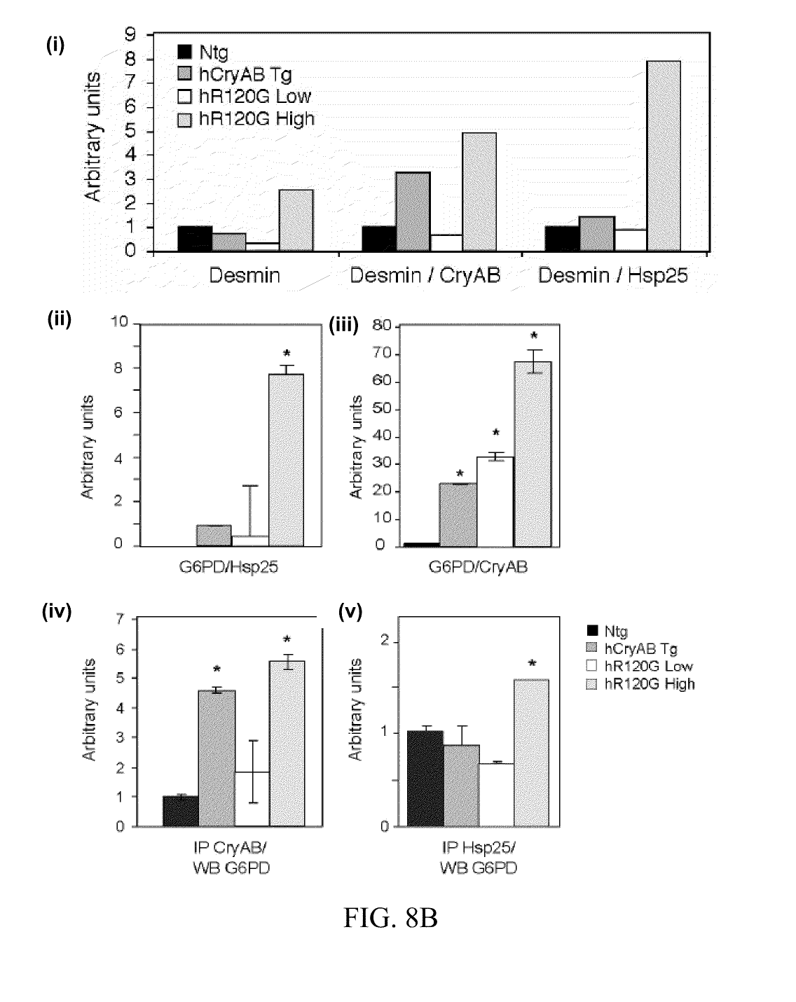

Additional posttranscriptional molecular mechanisms can account for hR120G-induced ˜2.0 fold G6PD enzyme activity and myocardial abundance (FIG. 6). Besides effects on its iin vitro chaperone activity and structural integrity (Perng, M. D., et al. 1999), hR120GCryAB per se exhibits increased binding for intermediate filaments (e.g. desmin) and effects on other client proteins including G6PD were unknown (Kumar, M. S., et al. 2005). To determine whether hR120GCryAB expression has direct consequences on molecular interactions involving the GSH biosynthetic pathway, reciprocal co-immunoprecipitations and immunoblot analysis were performed in heart homogenates. In hR120G High extracts, with anti-desmin and A6-desmin as associated with both CryAB and Hsp25 in pull-down experiments (FIG. 8). Likewise, immunoprecipitation with anti-G6PD antibody, and subsequent immunoblot analyses revealed interactions with CryAB and Hsp25 in hCryAB Tg, hR120G Low and hR120G High but not NTg, indicating CryAB overexpression per se increases certain protein-protein interactions of G6PD. The reciprocal experiment in which anti-CryAB was first used for immunoprecipitation followed by anti-G6PD immunodetection confirmed the molecular interactions in vivo (FIG. 8).

Because both Hsp25 and G6PD levels were increased in hR120G hearts, co-immunoprecipitation studies were performed to examine their possible molecular interactions in vivo. Reciprocal co-precipitation studies indicate insignificant interactions between G6PD and Hsp25 in heart extracts from NTg, hCryAB Tg and hR120G Low (FIG. 8). In contrast, similar co-immunoprecipitation of Hsp25 and G6PD proteins was exclusively dependent on hR120G High expression, indicating client protein effects between G6PD activity, and Hsp25 in vivo. These findings provide direct evidence that molecular interactions among members of the Hsp family and key antioxidative pathways are causally linked to the mechanisms promoting the pathogenesis of protein aggregation in hR120G cardiomyopathy in vivo.

G6PD Deficiency Decreases Pro-Reducing Shift and Abrogates Cardiac Hypertrophy in hR120G High Cardiomyopathic Mice:

As the rate-limiting enzyme of the pentose phosphate pathway, G6PD controls the production of reduced nicotinamide adenine-dinucleotide phosphate (NADPH), the principal source of reducing equivalents for GSH/GSSH recycling. If causal mechanisms are linked to marked upregulation of the G6PD, then maneuvers that either inhibit and/or down-regulate key members this molecular pathway should reverse redox imbalance triggering hR120G cardiomyopathy at high risk for heart failure. To test this hypothesis, male hemizygous G6PD-mutant mice (G6PD^{mut}, C3H background) were bred to hR120G High animals to generate double transgenic G6PD^{mut}/hR120G High mice.

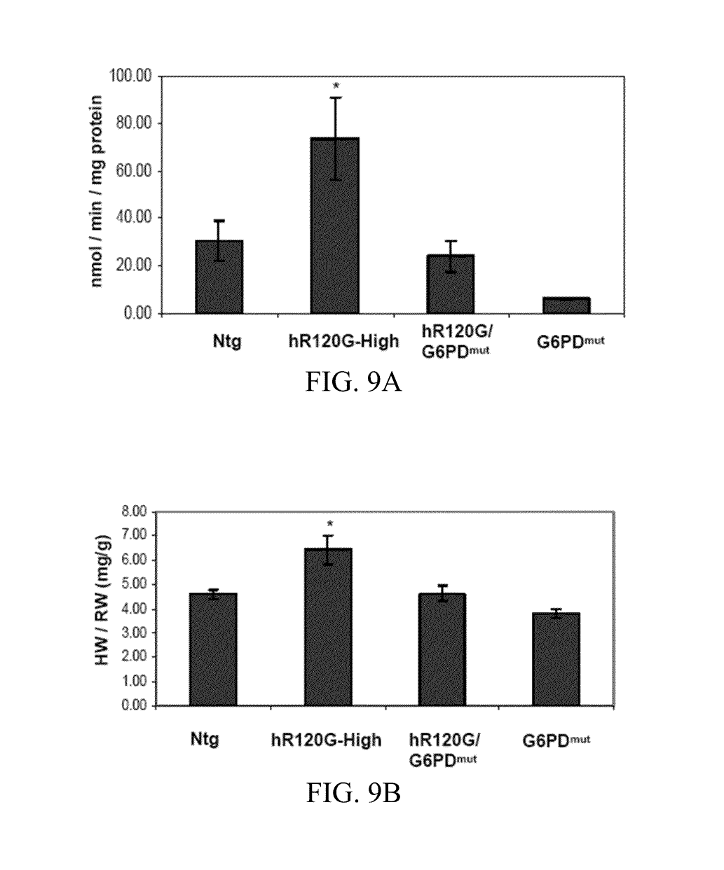

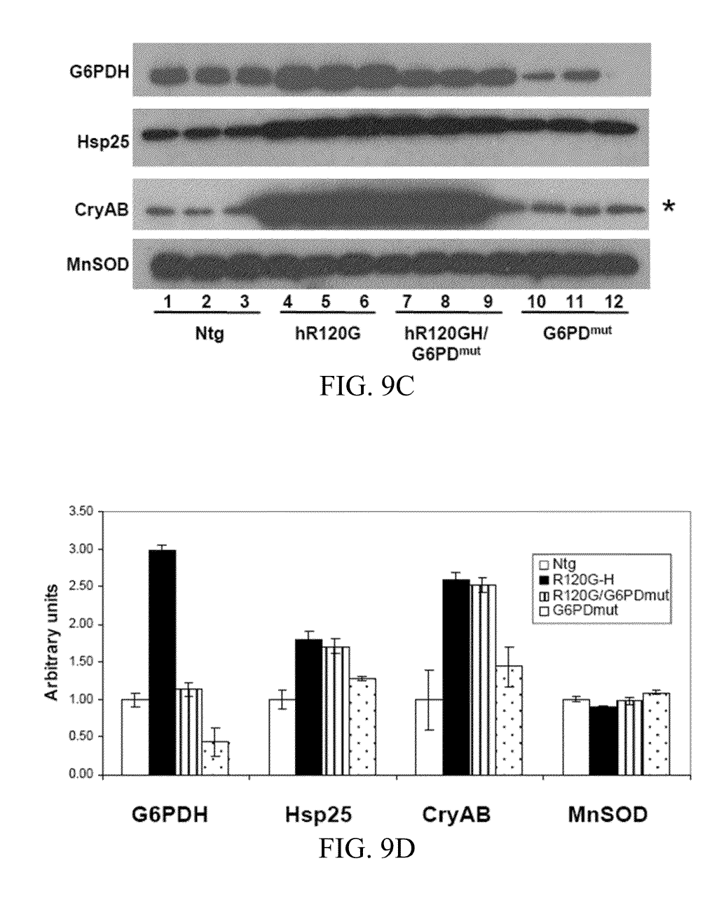

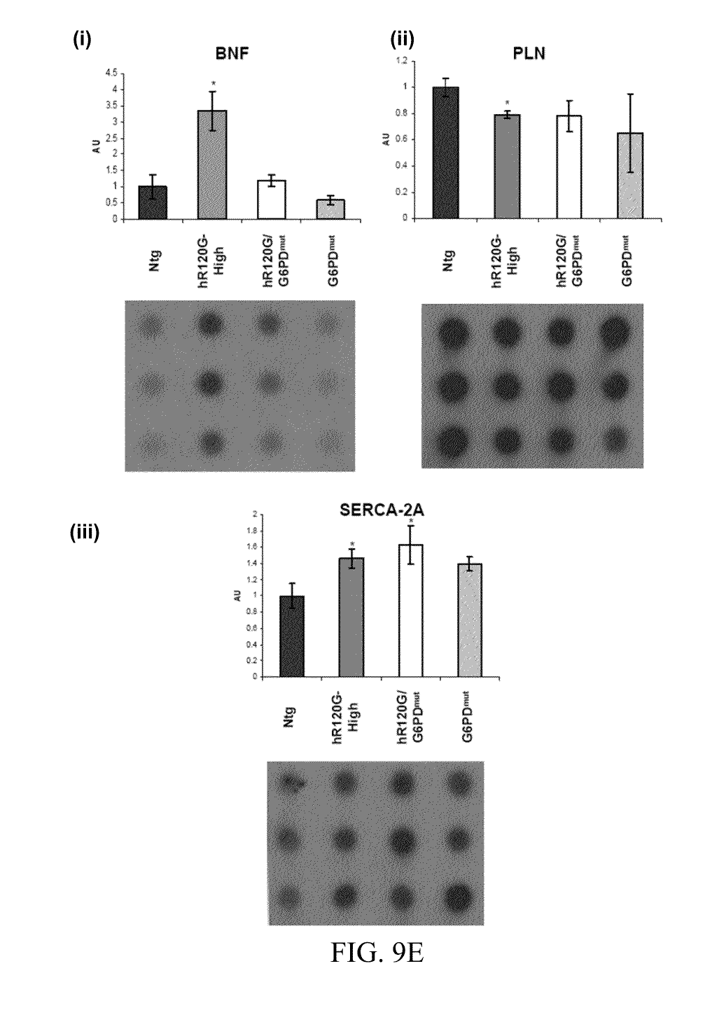

In age-matched experimental groups (˜6 months), FIG. 9A shows that the G6PD enzyme activity in hR120G High was ˜2.5-3.0-fold greater than either Ntg or hR120G/G6PD^{mut} (73.62±17.19 vs 30.69±8.14 or 23.98±6.84, p<0.05, respectively). Indeed, this modulation of G6PD enzyme activity in hR120G High/G6PD^{mut} was statistically not different from Ntg. Compared with NTg animals, the trend for greater GSH content, however, was modestly increased by 30% and 14% in hR120G High and double transgenic hR120G/G6PD^{mut}, respectively. Western blot studies revealed that G6PD protein content was significantly higher in hR120G High than NTg, hR120G/G6PD^{mut}, or G6PD^{mut} hearts (FIG. 9C, FIG. 9D). Moreover, the anticipated increases in total CryAB and Hsp25 protein levels were similar between in hR120G High and double transgenic hR120G High/G6PD^{mut}, indicating myocardial levels of total CryAB or Hsp25 expression induced by hR120G High was unaltered by G6PD deficiency in vivo. The expression of mitochondrial manganese superoxide dismutase was comparable among all experimental groups (FIG. 9C, FIG. 9D).

Lastly, cardiac hypertrophy is a sine quo non of hR120G High cardiomyopathy and a major risk for heart failure in experimental models and humans alike. Indeed, heart weight/body weight ratio of hR120G High was 33% greater than hR120G/G6PDdef (6.15±1.06 vs 4.63±0.27, p<0.05), the latter being similar to Ntg (4.63±0.27 vs 4.50±0.19, NS). Such profound effects in ameliorating the hypertrophic response in double-transgenic hR120G/G6PDdef hearts were confirmed at the molecular level using several biomarkers for cardiac hypertrophy (FIG. 9E). Therefore, the reversal in G6PD enzyme activity, lowering of GSH content, abrogation of cardiac hypertrophy in double-transgenic hR120G/G6PD^{mut} hearts demonstrate for the first time that G6PD plays a key role in production of reductive stress of the disease-causing hR120GCryAB mutation in mammals.

iii. Methods

Antibodies and Reagents:

The following antibodies and reagents were used: a polyclonal antibody, which recognizes both the mouse and human proteins, was raised against residues 164-175 of human CryAB. Rabbit anti-Hsp25, anti-Hsp70, anti-Hsp90 (StressGen, Victoria, BC, Canada) and rabbit anti-G6PD (Amersham Bio.), anti-catalase, anti-glutathione peroxidase, anti-glutathione reductase (AbCam), gamma-GCS/glutamate cysteine ligase-Ab 1 (Labvision, Neomarkers, CA) and Anti-DNP (Sigma Chemicals Co, St. Louis, Mo.) antibodies were purchased from commercial vendors. Acrylamide/bis-acrylamide, ammonium persulfate, protein assay reagent, protein standard markers (Bio-Rad, Richmond, Calif.) and enzymatic assay kits for reduced and oxidized glutathione, catalase, glutathione peroxidase, glutathione reductase were obtained from Bioxitech (Oxis Research). RNeasy, DNA purification kits (QIAGEN, Valenica, CA) and Northern Max kit (Ambion, Austin, Tex.), [α-³²P]dATP (Amersham) were obtained commercially.

Transgenic Constructs, Mouse Lines and Care:

The full-length human alpha-B crystallin (CryAB) was kindly provided by Dr. Goldman (Columbia University). The missense mutation, hR120G, was created from the human CryAB cDNA by PCR-based mutagenesis (Quick Change Site directed mutagenesis kit, Stratagene, LaJolla) and confirmed by sequencing. Subsequently, the cDNAs were placed under the control of alpha-myosin heavy chain (α-MHC) promoter (gift from Dr. Jeffrey Robbins, University of Cincinnatti, Ohio). Transgenic mice were generated by pronuclear injection according to standard procedure. Founders were identified by PCR and Southern blot and crossed with wild type C57/BL6 mice to establish the transgenic lines. Hemizygous mice for the capital-linked gene encoding G6PD with 20% of the normal enzymatic activity were obtained from Drs. Jane Leopold and Joseph Loscalzo at Boston University. Standard mouse breeding was used of generate compound R120G High/G6PD^{mut} heterozygotes. Mice were fed with standard diet and had access to water ad libidum; they were housed under controlled environment with 23±2° C. and 12-hour light/dark cycles. All experimental protocols followed the US Animal Welfare Acts and NIH guidelines and were approved by the University of Utah Animal Care and Use Committee.

Magnetic Resonance Imaging:

(MRI) was performed after animals were weighed and anesthetized with intraperitoneal injections of Avertin (2.5% tribromoethanol and 0.8% 2-methyl-2-butanol in water, Sigma Chemicals) and monitored for normal respiratory function. The MRI scan was performed using a 1.5 T Philips Gyroscan NT whole body imaging system (Philips Medical Systems). The mouse was positioned supine in a 15 cm petri dish and the electrocardiograph leads were attached to both front paws and one hindpaw. A standard finger coil was placed over the animal's chest and used for imagining the mouse heart. Heart rates were 380 to 450 beats per minute. Multislice, multiphase cine MRI was performed. Each study included a scout, coronal plane long axis of the left ventricle and a set of short axis acquisitions. Multiframe, short-axis gradient-echo sequences were used to measure LV end-systolic (LVESV) and diastolic volumes (LVEDV) as well as estimate LV mass and ejection fraction (EF). Four or five slices perpendicular to the long axis were obtained for each heart spanning from the apex to the base. The slice thickness was 1.6 mm with a 0.2 mm gap between slices. The pulse sequence was set for a heart rate of 210 bpm with nine cardiac phases and temporal resolution of 39 ms. The frame with the largest chamber dimensions was used as end diastole for mass and volume measurements and the image with the smallest chamber volume was used for end systolic measures. The LV mass, LVEDV, LVESV and EF were determined from images and calculated as previously described (Franco, F., et al. 1998; Franco, F., et al. 1999). Initial groups (n=10-15/group) of experimental animals were assessed serially at 3, and 6 and 10 months.

Dissociation of Adult Mouse Ventricular Myocytes:

Adult mouse myocyte isolation was performed with a modification of a previously described technique (Benjamin, I. J., et al. 1998). Briefly, hearts were removed from anesthetized mice and immediately attached to an aortic cannula. After perfusion with Ca²⁺-free modified Tyrode's solution for 5 minutes, hearts were digested with 0.25 mg/mL liberase blendzyme 1 (Roche Molecular Biochemicals) in 25 μmol/L CaCl₂-containing modified Tyrode's solution for 6-8 minutes. These two solutions consisted of (mmol/L) NaCl 126, KCl 4.4, MgCl₂ 1.0, NaHCO₃ 18, glucose 11, HEPES 4, with 0.13 U/mL insulin, and were gassed with 5% CO₂/95% O₂, which maintained the pH at 7.4. The digested hearts were removed from the cannula, and the left ventricles were cut into small pieces in 100 μmol/L Ca2+-containing modified Tyrode's solution. These pieces were gently agitated and then incubated in the same solution containing 2% albumin at 30° C. for 20 minutes. The cells were allowed to settle down with gravity. The supernatant was completely removed with a pipette and myocytes resuspended in 200 μmol/L Ca²⁺ and 2% albumin Tyrode's solution and allowed to settle for 20 minutes at 30° C. The cells were then resuspended in culture medium composed of 5% heat-inactivated fetal bovine serum (Hyclone), 47.5% MEM (GIBCO Laboratories), 47.5% modified Tyrode's solution, 10 mmol/L pyruvic acid, 4.0 mmol/L HEPES, and an additional 6.1 mmol/L glucose at 30° C. in a 5% CO₂ atmosphere. The percentage of normal rod-shape cells myocytes was determined by phase contrast microscopy after 1 hour incubation in culture medium at 30° C. in a 5% CO₂ atmosphere, and taken as an index of viability (Taylor, R. P., et al. 2005).

Methods for Isolated Heart Perfusion Studies:

Mice were anesthetized with an intraperitoneal injection of 50 mg/Kg body weight of sodium pentobarbital. Hearts were weighed and myocardial function was evaluated at 37° C. using an isolated Langendorff heart preparation as previously described (Neely, J. R., et al. 1967). The modified Krebs perfusion buffer contained (in mM): 10 glucose, 1.75 CaCl₂, 118.5 NaCl, 4.7 KCl, 1.2 MgSO₄, 24.7 NaHCO₃, 0.5 EDTA, 12 mU/mL Insulin, and was gassed with 95% O2-5% CO2. Afterload was set by an 104 cm high aortic column (ID 3.18 mm), and hearts were allowed to beat at their own intrinsic heart rate (HR) in a sealed water jacketed chamber maintained at 37° C. Hearts were initially perfused for 15 minutes with normal perfusate, then were switched to a perfusate solution containing 300 nM Dobutamine for 10 minutes to challenge the hearts as previously described (Arany, Z., et al. 2005), and finally returned to normal perfusate for the final 15 minutes of perfusion. An open-type catheter (20-gauge needle) was inserted into the left ventricle for determination of heart rate (HR) ventricular pressures (LVDP) and their derivatives (+/−dP/dt) with all data collected and analyzed at a sampling rate of 200 Hz using PowerLab (ADInstruments, Colorado Springs, Colo.). The data acquisition system was calibrated daily against a known column of perfusate at 0 mmHg and 80 mmHg A open-type catheter was chosen over an isovolumetric intraventricular balloon because of the small and varying size of the mouse heart and due to the fact that the open-type catheter has been shown to be as accurate as a balloon for determining changes in end-diastolic/developed pressure (Pahor, M., et al. 1985; Sutherland, F. J., et al. 2003.). Coronary flow (CF), normalized for heart wet weight, was determined by timed collection and cardiac external work (RPP) is defined as the product of HR and LVDP. At the end of the perfusion period the beating hearts were freeze—clamped and stored at −80° C. for further analysis.

Protein Isolation and Western Blot:

Hearts were harvested from animals and flash frozen in liquid nitrogen. Tissue was pulverized and homogenized in 25 mM HEPES, pH 7.4, 4 mM EDTA, 1.0 mM PMSF and Roche complete protease inhibitors. The extract was then centrifuged at 8,000-x g for 30 minutes at 4° C. The pellet was then resuspended in 20 mM Tris, pH 6.8, 1.0 mM EDTA and 1.0% SDS and briefly sonicated into solubilize. Protein concentrations for supernatant and pellet were determined using Bio-Rad protein assay kit. Equal amounts of protein extracts (10-20 μg) were loaded and separated by SDS-PAGE. The proteins were then transferred electrophoretically from the gels to Immobilon-P (Millipore) membrane. Blots were blocked in Tris Buffered Saline-Tween 20 (TBST) containing 5% (w/v) milk followed by incubation for 2 hrs with the respective primary antibody diluted in TBS buffer. Blots were then washed three times for 10 min each in TBST and incubated with anti-rabbit (1:25000)/mouse (1:10000) IgG horse radish peroxidase (Vector Labs), in TBS for 1 hr. After washing 5 times for 10 min each in TBS, the membranes were treated with ECL detection reagents (Amersham Bio) and the proteins were visualized by exposure to Blue sensitive biofilm (Hyblot Autoradiography, Denville Scientific, Inc.).

Ultrastructural Studies:

Fixation of the heart for electron microscopic studies was performed as described previously (Griffiths, G. 1993) using a series of retrograde perfusions with saline, followed by 1% glutaraldehyde/4% paraformaldehyde in 0.10 M sodium cacodylate buffer, pH 7.4. Following fixation, the heart was post-fixed in 1.0% osmium tetroxide, then dehydrated in increasing concentrations of alcohol, embedded in Epon-Araldite and sectioned with a diamond knife on a Reichert Jung Ultracut E microtome. Ultrathin sections were mounted on copper grids, stained with uranyl acetate and lead citrate, and examined at multiple magnifications with a JEOL 1200EX electron microscope.

Glutathione Measurements:

Hearts were dissected, atria and large vessels trimmed and rinsed briefly in PBS. Heart sections were weighed, flash frozen, pulverized and homogenized in 5% 5-sulphosalisilic acid (SSA). This solution was centrifuged, 10,000×g, at 4° C. for 10 minutes. The supernatant was removed and used for GSH assay. GSSG content was measured by using 100 μl fraction of the supernatant adding 2 μl of 2-vinylpyridine and 10 μl of 50% triethanolamine, which was allowed to stand at room temperature for 1 hour. Total glutathione and oxidized glutathione (samples derivatized with 2-vinyl pyridine) were measured by a standard recycling assay based on the reduction of 5,5-dithiobis-2-nitrobenzoic acid in the presence of glutathione reductase and NADPH (Griffith, O. W. 1980).

Protein Bound Thiols:

Protein-SSG levels were measured after sonicating and rinsing the protein pellets in 1.0% sulfosalicylic acid before resuspending in 0.01M Tris-HCl, pH 7.5. The samples were treated with 0.25% sodium borohydride at neutral pH for 45 minutes at 41° C. to reduce the sulphide links. Excess borohydride was removed by acidification and the released GSH was measured as described above.

Determination of Lipid Peroxides (TBARS):

Lipid peroxidation is a well-established mechanism of cellular injury and is used as an indicator of oxidative stress in cells and tissues in vivo. Lipid peroxides, mutagenic products derived from polyunsaturated fatty acids, are unstable and decompose to form complex compounds such as reactive carbonyl, the most abundant of which is malondialdehyde (MDA). The lipid peroxidation products, as MDA, were measured in the heart homogenates using the thiobarbituric acid (TBA) reaction (Esterbauer, H., et al. 1991). In brief, 2.0 ml of 20% TCA supernatants from heart homogenates were mixed with 1.0% TBA reagent and boiled in a water bath for 15 minutes. The absorbance of the chromogen produced was measured at 532 nm in a Beckman UV-visible spectrophotometer.

Immunochemical Quantitation of Protein Carbonyls:

Heart homogenates were prepared in 20 mM Tris-HCl buffer, pH 6.8 containing 0.2% SDS and treated with 10 mM Di-Nitro Phynyl Hydrazine (DNPH) as described previously (Yan, L. J., et al. 2000). The homogenates with DNPH were separated in 10% SDS-PAGE and probed against anti-DNPH antibody (Keller, R. J., et al. 1993; Shacter, E., et al. 1994). Nitrocellulose blots were incubated in 50 ml of 5% non-fat dried milk overnight at 4° C. and then washed with Tris-buffered saline (20 mM Tris, 500 mm NaCl pH 7.5), containing 0.1% Tween-20 (TBST), rinsed for 3 times (10 min each) and were incubated with primary rabbit anti-DNP antibody (1:2000 in TBST containing 0.2% BSA) for 2 hours at room temperature. Washes were repeated in TBST for 3 times before incubation with secondary rabbit IgG (diluted 1:25000 in TBST containing 0.2% BSA) for 1 hour at room temperature. After 5 washes (10 min each) in TBST, the blots were then treated with enhanced chemiluminescence (ECL, Amersham) detection kit. The signals for oxidized proteins were quantified using Image J densitometry software.

Glucose-6-Phosphate Dehydrogenase Activity:

Cytoplasmic extracts were prepared as described above and the supernatant was used to assess the G6PD activity (Lee, C. Y. 1982). Protein aliquots were prepared in 90-μM triethanolamine, pH 7.6, 10 mM MgCl2, 198 μM G-6-phosphogluconate and 100 μM NADP+. Similar reaction mixtures with 198 μM of glucose-6-phosphate were also prepared to measure the activity of 6-phospho gluconate dehydrogenase. The solutions were mixed and absorbance was read at 340 nm every 2 minutes for 20 minutes. The specific activity of glucose-6-phosphate dehydrogenase was determined by calculating the difference between the readings from the two reactions.

Antioxidant Enzyme Activity Assays:

To measure the cytosolic activities of selected antioxidant enzymes, Bioxitech (OxisResearch) kits were used. Catalase activity was determined using the Catalase-520™ assay in a two-step procedure (Aebi, H.1984). The rate of dismutation of hydrogen peroxide (H₂O₂) to water and molecular oxygen is proportional to the concentration of catalase. Diluted homogenates containing catalase were incubated in the presence of a known concentration of H₂O₂. After incubation for 60 seconds, the reaction was quenched with sodium azide. The amount of H₂O₂ remaining in the reaction mixture was then determined by the oxidative coupling reaction of 4-aminophenazone (4-aminoantipyrene, AAP) and 3,5-dichloro-2-hydroxybenzenesulfonic acid (DHBS) in the presence of H₂O₂ and catalyzed by horseradish peroxidase (HRP) and the resulting quinoneimine dye is measured at 520 nm.

The GPx-340™ assay is an indirect measure of the activity of cytosolic-GPx. Oxidized glutathione (GSSG), produced upon reduction of organic peroxide by c-GPx, is recycled to its reduced state by the enzyme glutathione reductase (GSH-R). The oxidation of NADPH to NADP+ is accompanied by a decrease in absorbance at 340 nm (A340) providing a spectrophotometric means for monitoring GPx enzyme activity. To assay c-GPx, tissue homogenate was added to a solution containing glutathione, glutathione reductase, and NADPH. The enzyme reaction was initiated by adding the substrate, tert-butyl hydroperoxide and the A340 was recorded. The rate of decrease in the A340 is directly proportional to the GPx activity in the sample. The GR-340 assay is based on the oxidation of NADPH to NADP+ catalyzed by a limiting concentration of glutathione reductase (Beutler, E. 1969). One unit GSH-R activity in the homogenates is defined as the amount of enzyme catalyzing the reduction of one micromole of GSSG per minute at pH 7.6 and 25° C. The reduction of GSSG, determined indirectly by the measurement of the consumption of NADPH, decreases the absorbance at 340 nm (A340) as a function of time.

Extraction of RNA and Northern Dot Blot Analyses:

Anesthetized animals were perfused in situ with 10 ml of sterile PBS and followed by 10 ml of RNA later solution before the hearts were immediately harvested, atria trimmed and the ventricle immersed in RNA later solution for 45 min at RT before frozen at −80° C. Total RNA was extracted and purified from 25-30 mg semi-dried frozen heart tissue using RNeasy mini kit (QIAGEN, Valencia, Calif.), according to the manufacturers instruction. RNA quality was monitored using Bio-analyzer/agarose gel electrophoresis. 1 μg of total RNA was suspended in Tris buffer, loaded and blotted on supercharged nylon membrane (BrighStar-plus, Ambion Inc.) using Biorad Biodot™ apparatus and the membrane was UV-cross linked in Strafalinhev. DNA probes for atrial natriuretic factor (ANF), brain natriuretic factor (BNF), CryAB and phospholamban (PLN) were generated using the following primer sets by PCR on mouse genomic DNA:

ANF (325 bp)
(SEQ ID NO: 8)
left, 5′ AACCTGCTAGACCACCTGGA-3′;
(SEQ ID NO: 9)
right, 5′ GGAAGCTGTTGCAGCCTAGT-3′;
BNF (237 bp),
(SEQ ID NO: 10)
left, 5′ CACTGAAGTTGTTGTAGGAAGACC-3′;
(SEQ ID NO: 11)
right, 5′ CAAAAGCAGGAAATACGCTATG-3′;
CryAB (300 bp),
(SEQ ID NO: 12)
left, 5′ TCATCTCCAGGGAGTTCCAC-3′;
(SEQ ID NO: 13)
right, 5′ TAATCTGGGCCAGCCCTTAG-3′;
and
Phospholamban (PLN, 583 bp),
(SEQ ID NO: 14)
left, 5′ GCTGCCAATTTCCTCAACAT-3′,
(SEQ ID NO: 15)
right, 5′ ATCACAGCCAACACAGCAAG-3′.

The respective PCR products from mouse genomic DNA were purified through 1.5% agarose gel electrophosesis and fragments were eluted using Qiaquick® gel extraction kit. The RNA blots were then probed with respective α-32P radio labeled DNA probes and hybridized in Ultrahyb (Ambion) solution for 16-18 hours and washed according to the manufacturer's instruction. Membranes were then exposed to a high radiosensitive X-ray film (Hyblot CL Autoradiography, Denville Scientific Inc.) for 16-24 hours and the hybridization signals were detected using autoradiography. The mRNA expression of the individual genes was scanned and quantified using Image J analysis software.

Morphological Analysis and Immunohistochemistry:

Hearts were removed and immersed in 4% paraformaldehyde overnight. Tissue was then processed and embedded in paraffin. Slides were heated to 60° C. for 30 minutes and de-paraffinized. The tissue was then permeabilized in 0.3% Triton-100, and incubated overnight with rabbit anti-Hsp25 (Stress-Gen). The following day sections were incubated with goat anti-rabbit FITC labeled antibody (Vector Labs).

Statistics:

Statistical analyses of data were performed by anlysis of variance (ANOVA) using SPSS software. Pairwise comparisons were made to study the significance between different groups using a Tukey's HSD post-hoc analysis. Data were expressed as mean±SD for >6 mice in each group. P values less than 0.05 were considered statistically significant.

TABLE 4
Heart, body and heart/body weight ratio (mg/g) for NTg, hCryAB Tg, hCryAb
R120G Low, hCryAB R120G High mice at 3 and 6 months of age.

| | 3 months | | | 6 months | | |
|---|---|---|---|---|---|---|
| | Heart wt | Body wt | | Heart wt | Body wt | |
| Groups | (mg) | (g) | HW/BW | (mg) | (g) | HW/BW |
| Non | 119.65 ± 9.65 | 25.67 ± 1.78 | 4.69 ± 0.44 | 131.03 ± 15.72 | 29.01 ± 3.35 | 4.60 ± 0.35 |
| transgenic | | | | | | |
| hCryBA | 119.75 ± 8.83 | 25.57 ± 1.87 | 4.73 ± 0.27 | 140.05 ± 15.46 | 32.23 ± 3.25 | 4.36 ± 0.48 |
| Wt Tg | | | | | | |
| hCryAB | 112.83 ± 13 | 26.6 ± 4.4 | 4.42 ± 0.3 | 128.28 ± 11.97 | 27.98 ± 2.79 | 4.61 ± 0.38 |
| R120G L | | | | | | |
| hCryAB | 126.75 ± 8.90 | 25.91 ± 2.21 | 5.03 ± 0.32 | 165.21 ± 11.70 | 27.68 ± 2.09 | 6.07 ± 0.56* |
| R120G H | | | | | | |

Data represent the mean ± SD for >15 animals.
*Heart weight and HW/BW ratio are significantly different in hR120GCryABHigh when compared to Ntg or other groups at 6 months,
P < 0.01.

TABLE 5
MRI data

| | Age | | | p value |
|---|---|---|---|---|
| Genotype | 3 months | 6 months | 10 months | intragroup |
| CryAB^{WT} | | | | |
| BW, g | 28.3 ± 2.02 | 30.06 ± 1.56 | 35.5 ± 2.28 | ND |
| LV mass, mg | 82.7 ± 2.82 | 93.2 ± 2.17 | 94.7 ± 3.36 | <0.02 |
| LV EDV, μl | 40.8 ± 2.89 | 39.4 ± 1.86 | 49.7 ± 2.17 | |
| LV ESV, μl | 16.1 ± 1.27 | 12 ± 2.15 | 19.2 ± 1.47 | |
| LV EF | 57.5 ± 3.24 | 67.8 ± 4.99 | 61.3 ± 3.75 | |
| n | 12 | 12 | 11 | |
| R120GCryABLow | | | | |
| BW, g | 24.4 ± 0.96 | 25.8 ± 2.55 | 35 ± 2.09 | ND |
| LV mass, mg | 78.3 ± 2.79 | 88.1 ± 2.81 | 101.6 ± 5.45 | <0.002 |
| LV EDV, μl | 41.5 ± 2.01 | 45.3 ± 2.17 | 55.1 ± 3.73 | <0.005 |
| LV ESV, μl | 15.3 ± 1.49 | 18.4 ± 2.21 | 23.7 ± 1.98 | <0.005 |
| LV EF | 64.6 ± 3.27 | 59.8 ± 4.24 | 57 ± 3.26 | |
| n | 16 | 12 | 7 | |
| R120GCryABHigh | | | | |
| BW, g | 24.4 ± 1.15 | 26.3 ± 0.92 | 35.2 ± 2.87 | ND |
| LV mass, mg | 88.3 ± 3.98 | 103.6 ± 4.77 | 129.8 ± 6.93^{a} | 0.0001 |
| LV EDV, μl | 41.8 ± 1.59 | 47.4 ± 2.93 | 61 ± 4.46 | <0.0005 |
| LV ESV, μl | 14.6 ± 1.26 | 21 ± 2.3993^{b} | 36.4 ± 7.46^{c} | <0.0005 |
| LV EF | 65.5 ± 2.19 | 54.2 ± 4.46 | 41.3 ± 9.2346^{d} | <0.02 |
| n | 12 | 8 | 5 | |

LV = left ventricle;
EDV = end-diastolic volume;
ESV end-systolic volume;
EF = ejection fraction;
ND = not determined.
^{a}indicates a significant difference between groups with a p value = 0.0002.
^{b}indicates a significant difference between groups with a p value = 0.005 at 6 months.
^{c}indicates a significant difference between groups with a p value = 0.01 at 10 months.
^{d}indicates a significant difference between groups with a p value = 0.02 at 10 months.

TABLE 6
Concentrations of reduced, oxidized glutathione and protein bound thiols (PSSG)
in heart tissue homogenates.

| Parameter/Groups | Non-transgenic | hCryAB Tg | hR120G Low | hR120G High |
|---|---|---|---|---|
| Total GSH | 811.19 ± 125.87 | 937.06 ± 97.90 | 1006.01 ± 58.74 | 1573.02 ± 33.57* |
| (nmol/mg | | | | |
| protein) (N = 6) | | | | |
| GSSG (nmol/mg | 18.20 ± 1.6 | 24.51 ± 1.7 | 24.01 ± 0.8 | 24.51 ± 0.9 |
| protein) (N = 6) | | | | |
| GSH/GSSH | 44.41 ± 3.0 | 38.11 ± 1.44 | 42.33 ± 2.54 | 64.43 ± 3.50* |
| PSSG (AU/mg | 1.0 ± 0.05 | 0.94 ± 0.13 | 0.87 ± 0.07 | 0.84 ± 0.01NS |
| protein) (N = 3) | | | | |

The total GSH content is increased without an effect on GSSG in hR120G High at 24 weeks of age. Heart tissue homogenates were prepared in 5% SSA and GSH was measured in the presence of NADPH and glutathione reductase. An aliquot of the homogenate was derivatized with vinyl pyridine to measure GSSG. The GSH/GSSG ratio is increased significantly in R120G High hearts at 6 months. Protein bound thiol groups were decomposed/removed by treating with sodium borohydrate and then GSH was measured in the supernatants.
Data represent the mean ± SD, of 3 experiments
*p < 0.05 when compared with the Ntg group

### 2. Example 2

### Global Expression Profiling Identifies Molecular Signatures During Early Onset of Protein Aggregation Cardiomyopathy in Mice

i. Methods

Transgenic Constructs and Mouse Lines:

Generation of transgenic mice is described elsewhere (Rajasekaran, N. S., et al. 2006). Briefly, the full-length human αB-crystallin (CryAB; αBC) was provided (Accession#S45360; Iwaki, A., et al. 1992). The missense mutation, R120G, was created from the human CryAB cDNA by PCR-based mutagenesis (Quick Change Site directed mutagenesis kit, Stratagene, La Jolla) and confirmed by sequencing. Subsequently, the cDNAs were placed under the control of α-myosin heavy chain (α-MHC) promoter. Transgenic mice were generated by pronuclear injection according to standard procedures. Founders were identified by polymerase chain reaction and Southern blot and then crossed with wild type C57BL/6 mice to establish the transgenic lines.

Phenotypic Characterization:

Before the gene array experiments, transthoracic echocardiography was performed on male, age-matched littermates of non-transgenic (NTG), human wild type CryAB (hCryAB^{WT}) transgenic and human mutant R120G CryAB (hR120GCryAB) transgenic mice to characterize cardiac function before tissue harvest. Experimental groups (n=5-15/group) of mice were consciously sedated and imaged in the left lateral decubitus position with a linear 13 MHz transducer (General Electric, Vivid V echocardiograph). Studies in conscious and anesthetized mice were compared and reproducible functional data was found with acceptable heart rates using an Isoflurane anesthetic regimen (Belke, D. D., et al. 2002). Digital images were obtained at a frame rate of 180/s. 2-dimensional images were recorded in parasternal long and short axis projections with guided m-mode recordings at the mid-ventricular level in both views. An average to 3-4 cardiac cycles were used for measurements. Thickness of the interventricular septum (IVSD), posterior wall (PWD) and internal LV dimensions in diastole (LVDD) and systole (LVSD) was measured for calculations of LV mass, relative wall thickness and LV fractional shortening (LVFS). LVEF was calculated with the Teichholz formula (Teichholz, L. E., et al. 1976).

RNA Isolation and Microarray Hybridization:

RNA was isolated from ventricles of 3- and 6-month old non-transgenic (NTG), human CryAB wild type transgenic (hCryAB WT) and human R120GCryAB transgenic (hR120GCryAB) mice.

Between 5-12 mice from each group were anesthetized and perfused in situ with 10 ml of sterile PBS before the hearts were immediately harvested, atria trimmed, and the ventricle immersed in RNA Later (Ambion, Austin, Tex.) solution for 45 min at room temperature before the samples were frozen at −80° C. Total RNA was extracted and purified from 25-30 mg heart tissue using the RNeasy mini kit (Qiagen, Valencia, Calif.) according to the manufacturer's instruction including the RNase-free DNase step. RNA quality was monitored by A260/A280 ratio, 1% agarose/formaldehyde gel electrophoresis and microfluid electrophoresis (Agilent Bioanalyzer, Agilent, Foster City Calif.). Labeling of RNA, microarray hybridization, scanning, and image processing were preformed by the Huntsman Cancer Institute Microarray Resource. Samples of between 4 and 7 biological replicates for each condition were hybridized to two microarray slides (“mouse A” and “mouse B”, with 9278 and 9047 mouse clones respectively, each printed in duplicate) made in-house, each consisting of a subset of the National Institute of Aging (NIA) 15K mouse clone set. All experimental samples were labeled with Cy3 dye and hybridized versus a standard reference sample (Universal Mouse Reference RNA, Stratagene, La Jolla, Calif.) labeled with Cy5 dye.

Microarray Data Analysis:

Microarray images where quantified using ImaGene software, version 6.0 (BioDiscovery, El Segundo Calif.). The raw, non-normalized data from the mouse A and mouse B spotted cDNA arrays were evaluated for overall quality with MVA and box plots to check for any intensity-dependent or spatial artifacts. The plots revealed subtle intensity-dependent and spatial variation that was corrected using LOWESS normalization with print-tip scope. Normalization was performed in the AROMA software (Bengtsson, H., et al. 2004). No background correction was performed. Poor quality spots were removed, and log ratios were calculated in AROMA. Data from the two microarrays for each sample were concatenated together to form a single data. Microarray experimental information and data were deposited in the Gene Expression Omnibus public database under accession number GSE9924.

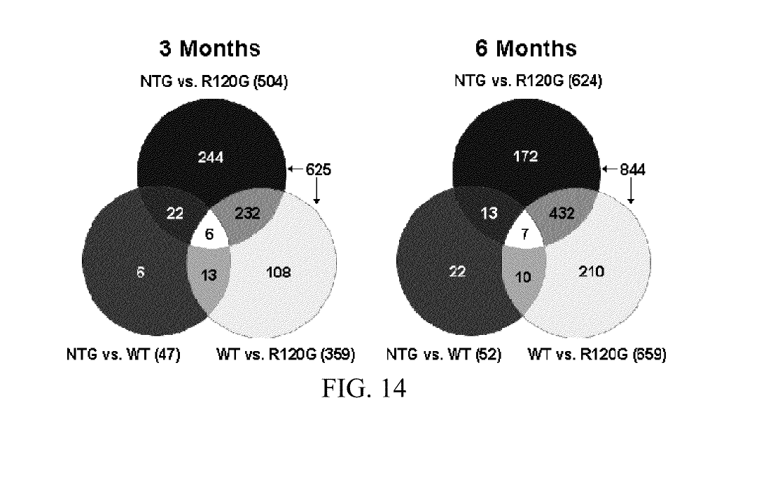

For each of the approximately 18,000 sequences (representing genes and ESTs) on the microarrays, the experimental versus reference standard intensity ratios were first calculated in order to find the mean averaged intensity ratio over all replicate spots for that sequence. Statistical analysis of differentially expressed genes and identification of potentially altered pathways were performed using GeneSifter software suite (VizX Labs, Seattle Wash., www.genesifter.net/web). Major expression differences in NTG, hCryAB^{WT} and hR120GCryAB mouse hearts were determined by separate ANOVA at 3- and 6 months. Expression of sequences was considered significantly altered if they had at least a two-fold change at an adjusted p-value of 0.005. The method of Benjamini and Hochberg was used to adjust for multiple comparisons by controlling the false discovery rate (“stringent analysis”). A second analysis was performed by pairwise comparisons for each combination (NTG vs. hCryAB^{WT}, NTG vs. hR120GCryAB, and hCryAB^{WT} vs. hR120GCryAB) at both 3- and 6 months. In this case, significance levels were set at a 1.5 fold cut-off at a Benjamini and Hochberg corrected p-value of 0.05 (“pairwise analysis”, see FIG. 14). Sequences with known gene function from the pairwise analyses were used to query the Kyoto Encyclopedia of Genes and Genomes (KEGG, www.genome.jp/kegg/ (Kanehisa, M., et al. 2006) annotated pathway database. Pathways represented in the dataset were filtered based on statistical z-scores of greater than 2.5 or less than −2.5.

Validation of Microarray Data by Northern Blotting:

For each condition, triplicate RNA samples used for microarray analysis were randomly chosen for Northern blot analysis. In brief, 10 μg of total RNA was loaded and separated on 1.0% agarose gel with formaldehyde, capillary transferred (Turbo Blotter, Whatman, Florham Park N.J.) on super charged nylon membrane (BrightStar-Plus, Ambion, Austin Tex.) and UV-cross linked. The agarose gels were imaged before transfer using an Image Station 2000R (Eastman Kodak, Rochester N.Y.) to monitor the 18s and 28s rRNA for loading normalization. cDNA probes were generated using their respective mouse clones by random priming in the presence of α-32P-ATP (Strip-EZ DNA, Ambion, Austin Tex.). Membranes were hybridized in Ultrahyb (Ambion, Austin Tex.) solution for 16-18 hours and washed in low (2×5 min, room temperature) and high (2×15 min, 68° C.) stringent solutions according to the manufacturer's instruction. Signals were detected using autoradiography and quantified using ImageJ software (National Institutes of Health, rsb.info.nih.gov/ij/).

ii. Results

A Mouse Model of Human R120GCryAB Protein Aggregation Myopathy:

The disease-causing missense mutation of human R120GCryAB was investigated by microarray analysis in attempts to identify the early molecular signatures of pathogenic significance underlying the cellular mechanisms of heart failure in transgenic mice. Among the anticipated cellular, molecular and morphological events of hR120GCryAB expression are decreased mutant protein degradation, protein aggregation, cardiac hypertrophy, heart failure and, ultimately, death by 16 months (Rajasekaran, N. S., et al. 2006). At 3 months, hR120GCryAB mice exhibited histological markers of protein aggregation, mimicking the phenotype described in patients. Therefore, the rationale for performing microarray analysis at 3- and 6 months is an attempt to identify molecular markers of key steps in disease progression from the compensation phase (i.e., normal cardiac function) and the transition towards heart failure (i.e., decreased contractile reserve), respectively. Indeed, the survival of hR120GCryAB mice declined significantly after 6 months (Rajasekaran, N. S., et al. 2006) indicating this time-point represents the transition between compensation and decompensation stages for this myopathic hR120GCryAB model. The compensation stage can include the absence of symptoms (shortness of breath, dyspnea on exertion, palpitations or signs of congestive heart failure (peripheral edema, pulmonary edema, increased heart rate or tachycardia). Noninvasive diagnostic studies can reveal normal or supranormal ejection fraction and cardiac hypertrophy. Sudden cardiac death remains an ominous complication that presents without any warning. The decompensated stage can include overt signs and symptoms of congestive heart failure, decreased ejection fraction, and intractable pump failure leading to death.

Cardiac Function:

All mice used for gene expression analyses in this study were first characterized for cardiac phenotype (Table 7). In 3 month groups old, a non-significant trend towards increased left ventricular ejection fraction (LVEF) was observed in hR120GCryAB mice (0.79±0.03) compared with hCryAB^{WT} and NTG controls (0.72±0.04 and 0.75±0.07, respectively). At the same age, however, left ventricular (LV) mass/body weight of hR120GCryAB (4.89±0.76 mg/G) was significantly greater than either hCryAB^{WT} or NTG mice (4.2±0.67 and 3.92±0.6, respectively). At 6 months, both the dimensions of diastolic and systolic cavities of hR120GCryAB (0.30±0.05 and 0.18±0.02 mm, respectively) were significantly decreased compared with hCryAB^{WT} and NTG (both 0.38±0.04 and 0.24±0.02 mm) Taken together, the findings of increased LV mass and reduced cavity dimensions are diagnostic for hypertrophic cardiomyopathy, establishing this phenotype for human R120GCryAB expression in genetically engineered mice. Although baseline cardiac function in hR120GCryAB mice remained in the normal range at 3 and 6 months, using magnetic resonance imaging (MRI) for serial assessment at 10 months, it was confirmed that depressed cardiac function and increased mortality ensues in similar cohorts of hR120GCryAB compared with either hCryAB^{WT} or NTG mice (Rajasekaran, N. S., et al. 2006).

TABLE 7
Phenotypic characterization of transgenic mice.

| | NTG | hCryAB^{WT} | | hR120GCryAB | |
|---|---|---|---|---|---|
| | 6 months | 3 months | 6 months | 3 months | 6 months |
| Parameter | (n = 5) | (n = 6) | (n = 6) | (n = 6) | (n = 15) |
| LVDD (mm) | 0.38 + 0.05 | 0.37 + 0.03 | 0.38 + 0.04 | 0.32 + 0.03*† | 0.30 + 0.04*† |
| LVSD (mm) | 0.24 + 0.05 | 0.24 + 0.03 | 0.24 + 0.03 | 0.19 + 0.02*† | 0.18 + 0.02*† |
| IVSD (mm) | 0.08 + 0.02 | 0.08 + 0.01 | 0.09 + 0.02 | 0.11 + 0.02*† | 0.12 + 0.02*† |
| PWD (mm) | 0.08 + 0.02 | 0.09 + 0.01 | 0.09 + 0.01 | 0.11 + 0.02 | 0.13 + 0.02*† |
| FS (%) | 38 + 6 | 35 + 3 | 36 + 2 | 40 + 3 | 40 + 4 |
| LVEF | 0.75 + 0.07 | 0.72 + 0.04 | 0.74 + 0.02 | 0.79 + 0.03 | 0.78 + 0.05 |
| HR (bpm) | 408 + 37 | 409 + 43 | 413 + 45 | 405 + 20 | 397 + 63 |
| ECHO LV/BW | 3.92 + 0.60 | 4.2 + 0.67 | 3.92 + 0.97 | 4.89 + 0.76*† | 5.07 + 1.13*† |
| (mg/g) | | | | | |
| AUTOPSY | 4.60 ± 0.35 | 4.73 + 0.27 | 4.36 ± 0.48 | 5.03 + 0.32*† | 6.07 + 0.56*† |
| HW/BW | | | | | |
| (mg/g) | | | | | |
| Relative Wall | 0.31 ± 0.12 | 0.31 ± 0.04 | 0.32 ± 0.03 | 0.45 ± 0.1*† | 0.57 ± 0.12*† |
| Thickness | | | | | |

Data are expressed in Mean + SD.
NTG = non-transgenic controls,
LVDD = left ventricular dimensions in diastole,
LVSD = left ventricular dimensions in systole,
IVSD = intraventricular septum dimension,
PWD = posterior wall dimension,
FS = factional shortening,
LVEF = left ventricular ejection fraction,
HR = heart rate in beats per minute, Heart weight (HW)/body weight (BW) ratios were measured using echocardiography (ECHO) and by direct measurement at autopsy.
*P < 0.05 hR120GCryAB vs. non-transgenic controls.
†P < 0.05 hR120GCryAB vs. hCryAB^{WT} (age matched).

TABLE 8
Pathway Analysis

| | | Genes | | | |
|---|---|---|---|---|---|
| | Genes | Identified | | z-score | |
| KEGG Pathway | on Array | Up | Down | Up | Down |
| 3 Month | | | | | |
| Up-regulated | | | | | |
| Glutathione metabolism | 18 | 5 | 0 | 6.44 | −0.49 |
| Antigen processing and | 24 | 5 | 2 | 5.38 | 3.02 |
| presentation | | | | | |
| Complement and coagulation | 25 | 4 | 0 | 4.02 | −0.58 |
| cascades | | | | | |
| 6 Month | | | | | |
| Up-regulated | | | | | |
| Antigen processing and | 24 | 6 | 1 | 5.92 | −0.05 |
| presentation | | | | | |
| Glutathione metabolism | 18 | 4 | 2 | 4.46 | 1.41 |
| Cell communication | 41 | 6 | 0 | 4.05 | −1.38 |
| Ribosome | 51 | 6 | 1 | 3.38 | −0.85 |
| Aminoacyl-tRNA biosynthesis | 23 | 3 | 0 | 2.59 | −1.03 |
| Down-regulated | | | | | |
| Oxidative phosphorylation | 75 | 1 | 19 | −1.00 | 9.09 |
| Fatty acid metabolism | 26 | 1 | 9 | 0.14 | 7.61 |
| Valine, leucine and isoleucine | 34 | 1 | 9 | −0.14 | 6.38 |
| degradation | | | | | |
| Carbon fixation | 17 | 1 | 6 | 0.58 | 6.28 |
| Pyruvate metabolism | 22 | 0 | 5 | −0.88 | 4.25 |
| Citrate cycle (TCA cycle) | 24 | 0 | 5 | −0.92 | 3.98 |
| Glycolysis/Gluconeogenesis | 31 | 1 | 5 | −0.04 | 3.24 |
| Alanine and aspartate | 15 | 1 | 3 | 0.71 | 2.98 |
| metabolism | | | | | |

Nontransgenic and hCryAB^{WT} Clusters Segregate from Mutant CryAB Expression:

To assess the overall reliability of the microarray intensity data, it was first asked whether the expression profiles from the experimental samples would partition into meaningful groups (“array clustering”). For this analysis, mean log intensity ratio information was used for all sequences on each array and applied hierarchical clustering with no filtering for statistical differences in expression (FIG. 11). Besides the age-matched non-transgenic (NTG) strain, hCryAB^{WT} was also included in order to independently assess the effects of protein overexpression per se compared with defective hR120GCryAB expression. Hierarchical clustering with no filtering for statistical differences in expression idnetified two main groups (FIG. 11): one consisting of the NTG and hCryAB^{WT} samples and the other corresponding to hR120GCryAB samples. Such robust alterations in gene expression were attributable to the presence of hR120GCryAB when compared with either NTG or hCryAB^{WT}. Two sub-clusters were evident within the hR120GCryAB main cluster, indicating a profound shift in hR120GCryAB-induced gene expression between 3 month and 6 months, corresponding to early and late compensation, respectively. The main cluster consisting of NTG and hCryAB^{WT} was less structured than the hR120GCryAB main cluster indicating that differences between these experimental groups were less robust. However, clustering of all six hCryAB^{WT} samples within one sub-cluster indicates that wild type transgene overexpression was sufficient to alter gene expression at 6 months (FIG. 11).

hR120GCryAB Triggers Marked Changes in Overall Gene Expression:

The 3- and 6 month datasets were next filtered for statistically significant changes in gene expression using ANOVA modeling. To identify the major gene expression changes, the search was arbitrarily restricted to those sequences exhibiting at least a two-fold change in expression. For this analysis, expression in hCryAB^{WT} and hR120GCryAB hearts was compared to expression in NTG hearts. With over 50,000 comparisons, it is remarkable that correction for multiple comparisons resulted in only a modest decrease in the number of identified sequences. At adjusted p-values less than 0.005, 95 and 114 sequences were identified as having significantly altered expression due to hR120GCryAB at 3 months and 6 months, respectively (Table 9). False positive rate of less than 1 sequence in 100 would be expected by this stringent analysis. Altered expression in the majority of these sequences could be attributed to the presence of the R120G mutation. Thus, wild type transgene overexpression per se had measurable but minimal effects, whereas the R120G mutation induced significant changes in gene expression.

Among the 95 sequences (68 up regulated, 27 down regulated) identified at 3 months, three sequences, Rpl9, Rpl10 and Slc14a1, had almost identical changes in expression in both the hCryAB WT and hR120GCryAB strains. Eight had their major effects in the hCryAB WT strain in which they were all upregulated (Rbm12b, Lrrc50 and 6 ESTs). The remaining sequences identified showed only modest changes in the 3-month-old hR120GCryAB mouse line. In contrast, 114 sequences were identified; 47 were up regulated and 67 down regulated in hearts of 6-month hR120GCryAB mice. Whereas wild type transgene overexpression per se had measurable but minimal effects, the R120G mutation induced significant changes in gene expression at both selected intervals.

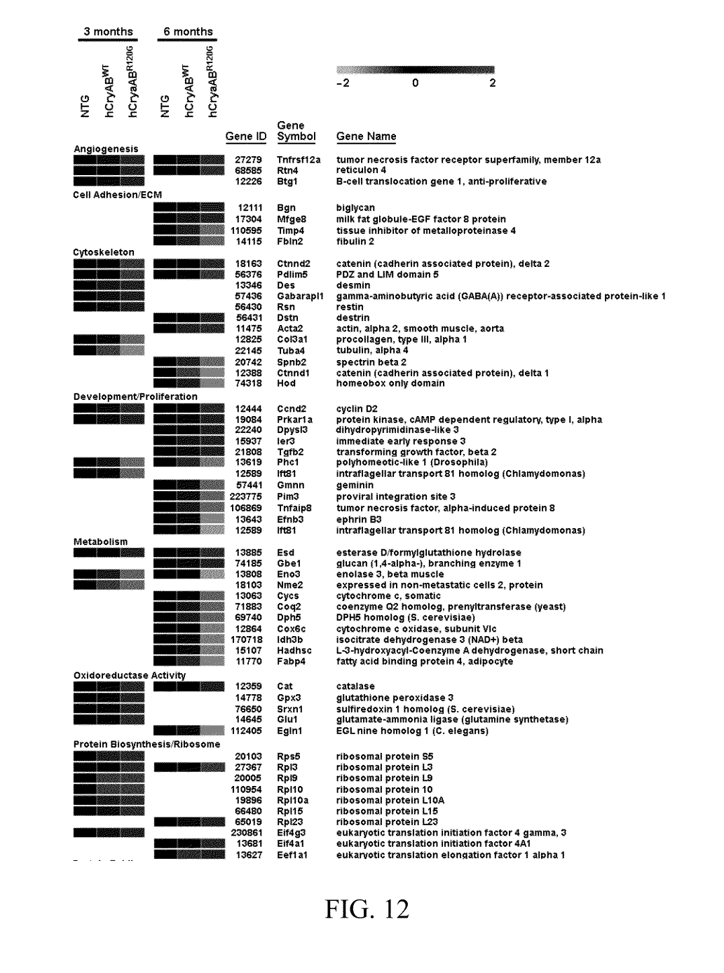

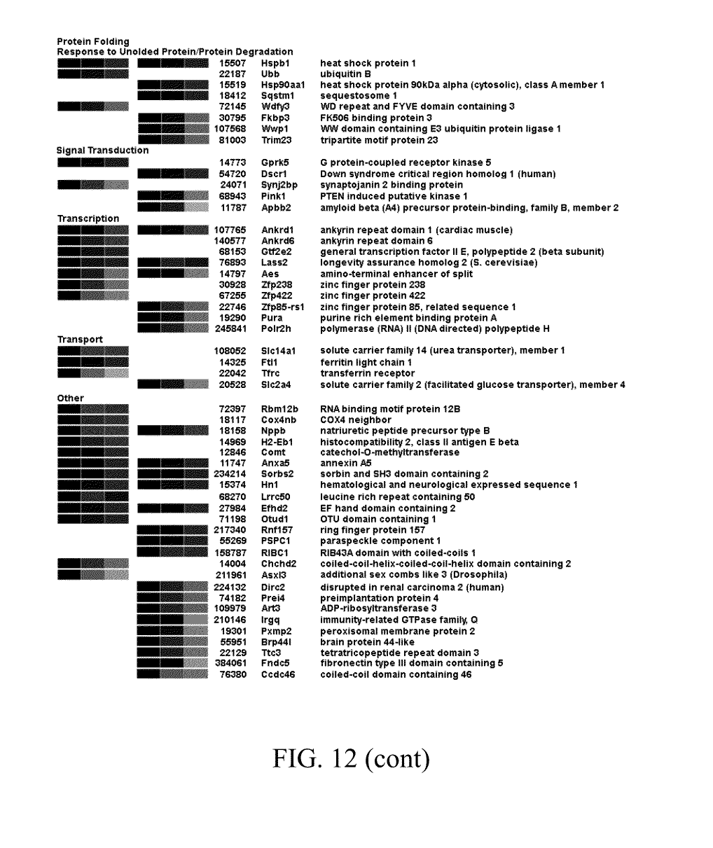

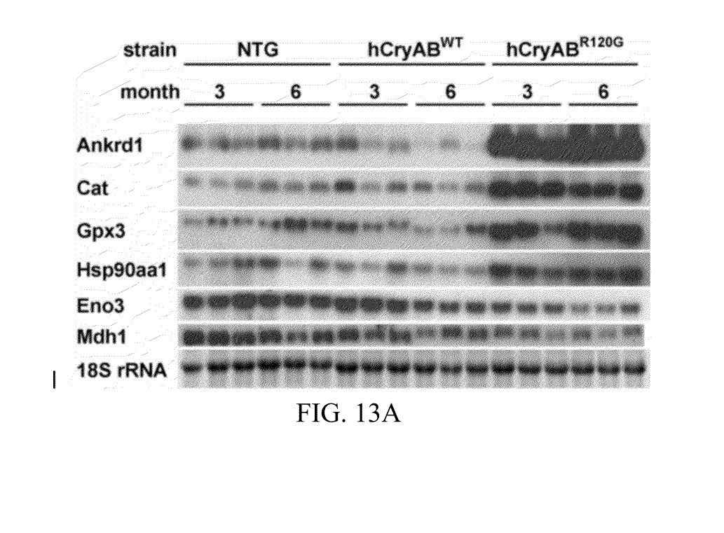

FIG. 12 shows the relative expression for each of the identified sequences with known functions. For the 209 sequences identified by ANOVA exhibiting a two-fold change in expression at an adjusted p-value <0.005, 142 sequences had meaningful names representing 109 unique genes (several genes were identified 2 or more times). Only 25 sequences (20 unique genes) were common to both the 3 and 6-month analyses. Consistent with cellular changes associated with protein aggregate myopathy, expression changes were noted for genes involved in stress response, cytoskeletal structure, protein synthesis, protein folding, and protein degradation.

hR120GCryAB Expression Defines a Subset of Cellular Pathways:

Because mutant transgene overexpression likely imparts specific consequences on myocardial dysfunction through re-programming of intracellular regulatory mechanisms, the dataset was next examined for significantly altered gene expression of cellular pathways related to cardiotoxicity. 625 and 844 sequences were found, which yielded at least a 1.5 fold expression change, as having significantly altered expression attributable to hR120GCryAB overexpression at 3 and 6 months, respectively, at an adjusted p-value 0.05 (FIG. 14). Known genes from these sequences were then used to query an annotated database and to define interdependent networks among biological processes.

At 3 months, several genes encoding major classes of heat-shock proteins and molecular chaperones (the majority of genes identified in the “antigen processing and presentation” pathway) were significantly upregulated (Tables 7 and 11). Whereas heat-shock protein genes (the majority of genes identified in the “antigen processing and presentation” pathway) encoding major classes of heat shock proteins and molecular chaperones (Table 10) were upregulated at 3 months, catabolic control for glutathione metabolism and complement and coagulation cascades were found among the ‘early response’ pathways. In spite of numerous genes that were down-regulated at 3 months, no distinct pathways were identified at the chosen z-score threshold.

Accompanying the anticipated reprogramming and remodeling from disease progression and the transition towards heart failure, 13 pathways were identified at 6 months (Tables 13 and 10). Both glutathione metabolism and antigen processing and presentation were upregulated along with three additional new pathways engaged in cell communication, ribosomal biosynthesis, and aminoacyl-tRNA biosynthesis. Genes encoding cellular communication pathway were limited to cytoskeletal and extra-cellular matrix proteins such as collagen, beta-actin, desmin and lamin A indicating cellular and tissue restructuring were characteristics of cardiomyopathy. In this regards, up regulation of ribosomal components, aminoacyl-tRNA biosynthetic pathways and down regulation of tRNA degradation enzymes indicate a concomitant demand for protein synthesis. Among down regulated pathways at 6 months, the most dramatic changes were noted for the oxidative phosphorylation and fatty acid metabolism pathways as were pathways for multiple stages of intermediate metabolism, indicating a deficiency in metabolic precursor molecules and, perhaps, myocardial energetics.

Validation by Northern Blot Analysis:

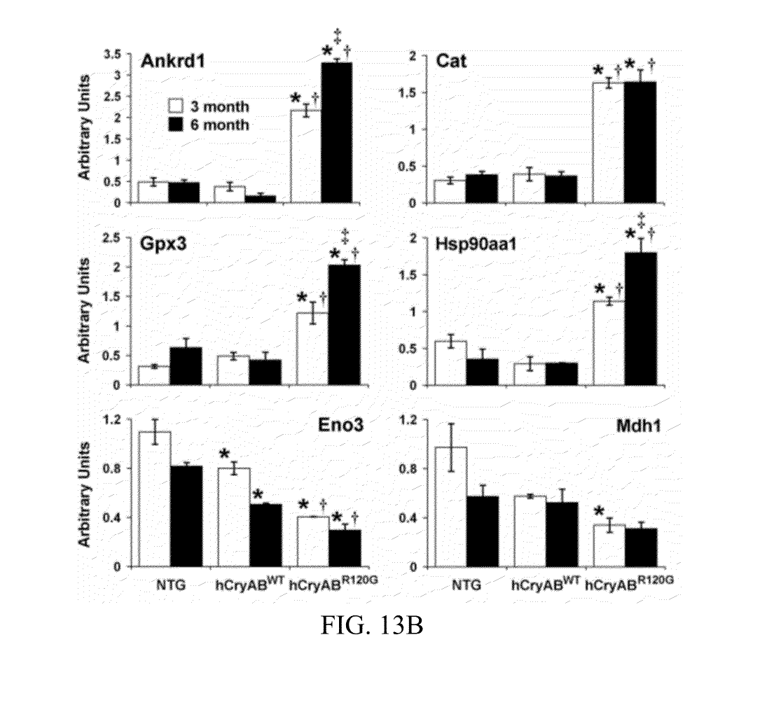

Next, the array expression was validated by Northern blot analysis of six representative genes identified as either upregulated (ankyrin repeat domain 1 (cardiac muscle), catalase, glutathione peroxidase 3, heat shock protein 90 kda alpha (cytosolic), class A member 1), or down-regulated (enolase 3, beta muscle, malate dehydrogenase 1, NAD (soluble)) by the microarray analysis (FIG. 13, Table 13). All transcripts showed remarkable concordance with the microarray data for both magnitude and direction. Ankrd1, Cat and Eno3, which were identified by the pairwise analysis, had significantly altered expression at both 3- and 6 months. Whereas Gpx3 was upregulated at 3 months only, Hsp90aa1, identified by the stringent analysis, was upregulated at 6 months only. Mdh1 was not identified as significantly altered in the stringent analysis. For additional validation of the cDNA microarray data, expression was examined in two samples each from 6-month hR120GCryAB and hCryAB^{WT} hearts using an oligonucleotide-based microarray platform (Affymetrix). Similar expression changes in the 109 unique genes identified by ANOVA were seen for both platforms (Table 13).

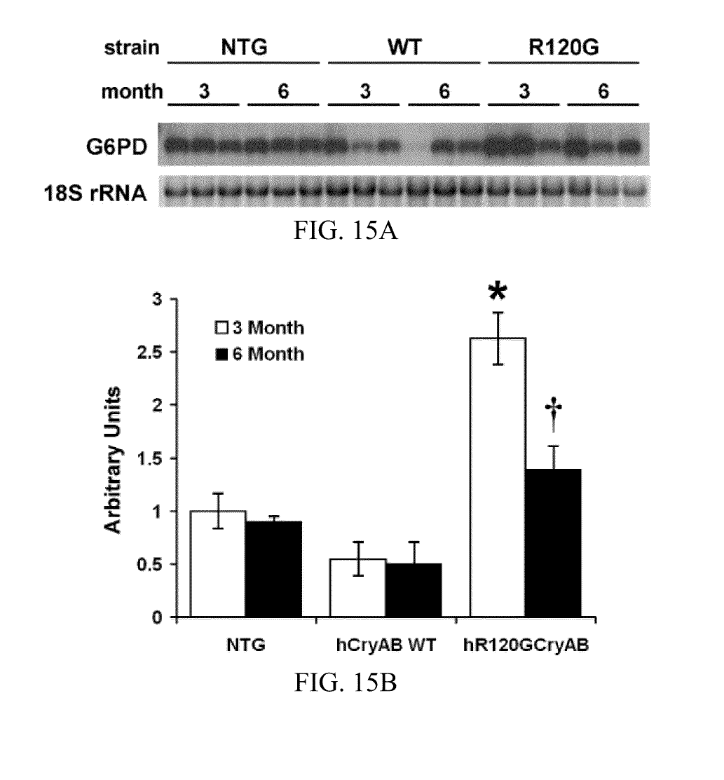

The microarray analysis did not identify G6PD mRNA as being upregulated in R120G hearts, whereas previous analysis demonstrated that G6PD protein was elevated about 4-fold relative to control tissue. G6PD was represented on the microarray by a single cDNA clone. Northern blot analysis using a probe generated from this cDNA clone was unsuccessful. However, using a second probe, representing exon 13 of the G6PD gene, Northern blot analysis showed a 4.8 and 2.8 fold increase in G6PD mRNA expression at 3 months and 6 months respectively (FIG. 15).

TABLE 9
Results of ANOVA Analysis.
Number of Sequences Identified

| 3 Months | 6 Months | Correction for Multiple Comparisons |
|---|---|---|
| 127 | 151 | P < 0.05 (uncorrected) |
| 126 | 151 | P < 0.05 (adjusted) |
| 108 | 126 | P < 0.01 (adjusted) |
| 95 | 114 | P < 0.005 (adjusted) |

The number of sequences with altered expression in hCryAB^{WT}, hR120GCryAB or both hCryAB^{WT} and hR120GCryAB relative to the NTG control are listed. Adjusted P-values were determined by controlling the false discovery rate.

TABLE 10
Gene lists for pathway analyses.

| | | | | | |
|---|---|---|---|---|---|
| | Gene | Gene | | | |
| Accession | Symbol | ID | Ratio | Direction | Gene Name |

3 month
Glutathione metabolism

| | | | | | |
|---|---|---|---|---|---|
| BG065030 | Gpx1 | 14775 | 1.61 | up | glutathione peroxidase 1 |
| BG073718 | Gpx3 | 14778 | 2.42 | up | glutathione peroxidase 3 |
| BG073190 | Gsta4 | 14860 | 1.59 | up | glutathione S-transferase, alpha 4 |
| BG086970 | Gstm1 | 14862 | 1.70 | up | glutathione S-transferase, mu 1 |
| BG074397 | Gstm1 | 14862 | 1.79 | up | glutathione S-transferase, mu 1 |
| BG086330 | Mgst1 | 56615 | 1.58 | up | microsomal glutathione S-transferase 1 |

Antigen processing and presentation

| | | | | | |
|---|---|---|---|---|---|
| BG078496 | Ctsl | 13039 | 1.60 | up | cathepsin L |
| BG077017 | H2-Eb1 | 14969 | 4.07 | up | histocompatibility 2, class II antigen |
| | | | | | E beta |
| BG078795 | Hspa5 | 14828 | 2.12 | down | heat shock 70 kD protein 5 (glucose- |
| | | | | | regulated protein) |
| BG064772 | Hspca | 15519 | 1.62 | up | heat shock protein 90 kDa alpha |
| | | | | | (cytosolic), class A member 1 |
| BG074109 | Hspca | 15519 | 1.76 | up | heat shock protein 90 kDa alpha |
| | | | | | (cytosolic), class A member 1 |
| BG064774 | Hspca | 15519 | 1.70 | up | heat shock protein 90 kDa alpha |
| | | | | | (cytosolic), class A member 1 |
| BG088007 | Hspcb | 15516 | 1.97 | up | heat shock protein 90 kDa alpha |
| | | | | | (cytosolic), class B member 1 |
| BG079631 | Hspcb | 15516 | 1.70 | up | heat shock protein 90 kDa alpha |
| | | | | | (cytosolic), class B member 1 |
| BQ550275 | Ii | 16149 | 1.73 | up | CD74 antigen (invariant polypeptide |
| | | | | | of major histocompatibility complex, |
| | | | | | class II antigen-associated) |
| BG073636 | Psme1 | 19186 | 1.50 | down | proteasome (prosome, macropain) 28 |
| | | | | | subunit, alpha |

Complement and coagulation cascades

| | | | | | |
|---|---|---|---|---|---|
| BG074814 | C1qa | 12259 | 2.02 | up | complement component 1, q |
| | | | | | subcomponent, alpha polypeptide |
| BG087868 | C1qb | 12260 | 1.96 | up | complement component 1, q |
| | | | | | subcomponent, beta polypeptide |
| AW547306 | C1qg | 12262 | 1.70 | up | complement component 1, q |
| | | | | | subcomponent, C chain |
| BQ553183 | F13a1 | 74145 | 1.70 | up | coagulation factor XIII, A1 subunit |

6-month - Up-regulated
Ribosome

| | | | | | |
|---|---|---|---|---|---|
| BG085976 | Rpl10 | 110954 | 1.73 | up | ribosomal protein 10 |
| BG072592 | Rpl3 | 27367 | 2.03 | up | ribosomal protein L3 |
| BG079511 | Rpl3 | 27367 | 1.96 | up | ribosomal protein L3 |
| BG072595 | Rpl3 | 27367 | 2.64 | up | ribosomal protein L3 |
| BG074107 | Rpl3 | 27367 | 2.31 | up | ribosomal protein L3 |
| BG072985 | Rpl7 | 19989 | 1.77 | up | ribosomal protein L7 |
| BG063847 | Rpl8 | 26961 | 1.74 | down | ribosomal protein L8 |
| BG085624 | Rps4x | 20102 | 1.77 | up | ribosomal protein S4, X-linked |
| BG072598 | Rps5 | 20103 | 1.51 | up | ribosomal protein S5 |
| BG072600 | Rps5 | 20103 | 1.58 | up | ribosomal protein S5 |
| AW556153 | Rps6 | 20104 | 1.53 | up | ribosomal protein S6 |

Glutathione metabolism

| | | | | | |
|---|---|---|---|---|---|
| BG073718 | Gpx3 | 14778 | 1.58 | up | glutathione peroxidase 3 |
| BG073190 | Gsta4 | 14860 | 1.68 | up | glutathione S-transferase, alpha 4 |
| BG074397 | Gstm1 | 14862 | 1.59 | up | glutathione S-transferase, mu 1 |
| Bg086330 | Mgst1 | 56615 | 1.60 | up | microsomal glutathione S-transferase 1 |
| BG088778 | Mgst3 | 66447 | 1.59 | down | microsomal glutathione S-transferase 3 |
| BG072517 | Gstm7 | 68312 | 1.69 | down | glutathione S-transferase, mu 7 |

Antigen processing and presentation

| | | | | | |
|---|---|---|---|---|---|
| BG078496 | Ctsl | 13039 | 1.68 | up | cathepsin L |
| BG08416 | Grp58 | 14827 | 1.68 | up | protein disulfide isomerase |
| | | | | | associated 3 |
| BG077017 | H2-Eb1 | 14969 | 2.05 | up | histocompatibility 2, class II antigen |
| | | | | | E beta |
| BG064772 | Hspca | 15519 | 2.43 | up | heat shock protein 90 kDa alpha |
| | | | | | (cytosolic), class A member 1 |
| BG074109 | Hspca | 15519 | 2.86 | up | heat shock protein 90 kDa alpha |
| | | | | | (cytosolic), class A member 1 |
| BG064774 | Hspca | 15519 | 1.77 | up | heat shock protein 90 kDa alpha |
| | | | | | (cytosolic), class A member 1 |
| BG063605 | Hspca | 15519 | 2.41 | up | heat shock protein 90 kDa alpha |
| | | | | | (cytosolic), class A member 1 |
| BG088007 | Hspcb | 15516 | 1.87 | up | heat shock protein 90 kDa alpha |
| | | | | | (cytosolic), class B member 1 |
| BG067038 | Hspcb | 15516 | 1.58 | up | heat shock protein 90 kDa alpha |
| | | | | | (cytosolic), class B member 1 |
| BG079631 | Hspcb | 15516 | 1.64 | up | heat shock protein 90 kDa alpha |
| | | | | | (cytosolic), class B member 1 |
| BQ550275 | Ii | 16149 | 1.59 | up | CD74 antigen (invariant polypeptide |
| | | | | | of major histocompatibility complex, |
| | | | | | class II antigen-associated) |
| BG073636 | Psme1 | 19186 | 1.51 | down | proteasome (prosome, macropain) 28 |
| | | | | | subunit, alpha |
| BG066650 | Psme1 | 19186 | 1.51 | down | proteasome (prosome, macropain) 28 |
| | | | | | subunit, alpha |

Cell communication

| | | | | | |
|---|---|---|---|---|---|
| BG063870 | Actb | 11461 | 1.83 | up | actin, beta, cytoplasmic |
| C78835 | Actb | 11461 | 1.62 | up | actin, beta, cytoplasmic |
| BG077677 | Actb | 11461 | 2.08 | up | actin, beta, cytoplasmic |
| BG063722 | Actb | 11461 | 1.51 | up | actin, beta, cytoplasmic |
| BG073735 | Col1a2 | 12843 | 1.81 | up | procollagen, type I, alpha 2 |
| BG074327 | Col3a1 | 12825 | 2.09 | up | procollagen, type III, alpha 1 |
| BG086357 | Col3a1 | 12825 | 2.18 | up | procollagen, type III, alpha 1 |
| BG085576 | Col3a1 | 12825 | 2.10 | up | procollagen, type III, alpha 1 |
| BG088953 | Col4a1 | 12826 | 1.54 | up | procollagen, type IV, alpha 1 |
| BQ554492 | Des | 13346 | 1.62 | up | desmin |
| BG077621 | Lmna | 16905 | 1.52 | up | lamin A |

Aminoacyl-tRNA biosynthesis

| | | | | | |
|---|---|---|---|---|---|
| C77246 | Yars | 107271 | 1.69 | up | tyrosyl-tRNA synthetase |
| BG079401 | Nars | 70223 | 1.51 | up | asparaginyl-tRNA synthetase |
| BG079434 | Sars1 | 20226 | 1.53 | up | seryl-aminoacyl-tRNA synthetase |

6-month - Down-regulate
Oxidative phosphorylation

| | | | | | |
|---|---|---|---|---|---|
| BG069853 | Uqcr | 66594 | 1.52 | down | ubiquinol-cytochrome c reductase |
| | | | | | (6.4 kD) subunit |
| BG086273 | Grim19 | 67184 | 1.56 | down | NADH dehydrogenase (ubiquinone) |
| | | | | | 1 alpha subcomplex, 13 |
| BQ553743 | Uqcrfs1 | 66694 | 1.55 | down | ubiquinol-cytochrome c reductase, |
| | | | | | Rieske iron-sulfur polypeptide 1 |
| BG074111 | Atp5c1 | 11949 | 1.83 | down | ATP synthase, H+ transporting, |
| | | | | | mitochondrial F1 complex, gamma |
| | | | | | polypeptide 1 |
| BG088873 | Atp5e | 67126 | 1.55 | down | ATP synthase, H+ transporting, |
| | | | | | mitochondrial F1 complex, epsilon |
| | | | | | subunit |
| BG063439 | Atp5h | 71679 | 1.51 | down | ATP synthase, H+ transporting, |
| | | | | | mitochondrial F0 complex, subunit d |
| BG072826 | Atp5l | 27425 | 1.71 | down | ATP synthase, H+ transporting, |
| | | | | | mitochondrial F0 complex, subunit g |
| BG086960 | Atp6v1h | 108664 | 1.61 | up | ATPase, H+ transporting, lysosomal |
| | | | | | V1 subunit H |
| BG076562 | Ndufb7 | 66916 | 1.52 | down | NADH dehydrogenase (ubiquinone) |
| | | | | | 1 beta subcomplex, 7 |
| BG064317 | Cox7b | 66142 | 1.73 | down | cytochrome c oxidase subunit VIIb |
| BG077969 | Ndufs8 | 225887 | 1.66 | down | NADH dehydrogenase (ubiquinone) |
| | | | | | Fe—S protein 8 |
| BG075903 | Ndufc1 | 66377 | 1.54 | down | NADH dehydrogenase (ubiquinone) |
| | | | | | 1, subcomplex unknown, 1 |
| BQ554627 | Ndufa4 | 17992 | 1.69 | down | NADH dehydrogenase (ubiquinone) |
| | | | | | 1 alpha subcomplex, 4 |
| BG086348 | Ndufb10 | 68342 | 1.64 | down | NADH dehydrogenase (ubiquinone) |
| | | | | | 1 beta subcomplex, 10 |
| BG087636 | Ndufb9 | 66218 | 1.65 | down | NADH dehydrogenase (ubiquinone) |
| | | | | | 1 beta subcomplex, 9 |
| BG066265 | Ndufs4 | 17993 | 1.65 | down | NADH dehydrogenase (ubiquinone) |
| | | | | | Fe—S protein 4 |
| BG072514 | Np15 | 104130 | 1.53 | down | |
| BG084240 | Ndufs7 | 75406 | 1.69 | down | NADH dehydrogenase (ubiquinone) |
| | | | | | Fe—S protein 7 |
| BG086026 | Sdha | 66945 | 1.86 | down | succinate dehydrogenase complex, |
| | | | | | subunit A, flavoprotein (Fp) |
| BG069853 | Uqcrc1 | 22273 | 1.58 | down | ubiquinol-cytochrome c reductase |
| | | | | | core protein 1 |

Fatty acid metabolism

| | | | | | |
|---|---|---|---|---|---|
| BG065314 | Acadm | 11364 | 1.70 | down | acetyl-Coenzyme A dehydrogenase, |
| | | | | | medium chain |
| BG065033 | Acads | 11409 | 1.51 | down | acyl-Coenzyme A dehydrogenase, |
| | | | | | short chain |
| BG083405 | Acat1 | 110446 | 1.52 | down | acetyl-CoA acetyltransferase |
| BG073167 | Hsd17b4 | 15488 | 2.01 | up | hydroxysteroid (17-beta) |
| | | | | | dehydrogenase 4 |
| BG069423 | Dci | 13177 | 2.17 | down | dodecenoyl-Coenzyme A delta |
| | | | | | isomerase (3,2 trans-enoyl-Coenyme |
| | | | | | A isomerase) |
| BG079992 | Echs1 | 93747 | 2.01 | down | enoyl Coenzyme A hydratase, short |
| | | | | | chain, 1, mitochondrial |
| BG074754 | Acsl1 | 14081 | 1.56 | down | acyl-CoA synthetase long-chain |
| | | | | | family member 1 |
| BG087380 | Hadha | 97212 | 1.63 | down | hydroxyacyl-Coenzyme A |
| | | | | | dehydrogenase/3-ketoacyl- |
| | | | | | Coenzyme A thiolase/enoyl- |
| | | | | | Coenzyme A hydratase (trifunctional |
| | | | | | protein), alpha subunit |
| BG086728 | Hadhsc | 15107 | 2.33 | down | L-3-hydroxyacyl-Coenzyme A |
| | | | | | dehydrogenase, short chain |

Carbon fixation

| | | | | | |
|---|---|---|---|---|---|
| BG067706 | Gpt2 | 108682 | 1.69 | down | glutamic pyruvate transaminase |
| | | | | | (alanine aminotransferase) 2 |
| BG082516 | Fbp2 | 14120 | 1.54 | down | fructose bisphosphatase 2 |
| BG078765 | Got2 | 14719 | 1.66 | down | glutamate oxaloacetate transaminase |
| | | | | | 2, mitochondrial |
| BQ550883 | Mdh2 | 17448 | 1.57 | down | malate dehydrogenase 2, NAD |
| | | | | | (mitochondrial) |
| BG067227 | Mdh1 | 17449 | 2.00 | down | malate dehydrogenase 1, NAD |
| | | | | | (soluble) |
| BG064747 | Gpt1 | 76282 | 1.85 | down | glutamic pyruvic transaminase 1, |
| | | | | | soluble |
| BG075310 | Tkt | 21881 | 1.56 | up | transketolase |

Valine, leucine and isoleucine degradation

| | | | | | |
|---|---|---|---|---|---|
| BG065314 | Acadm | 11364 | 1.70 | down | acetyl-Coenzyme A dehydrogenase, |
| | | | | | medium chain |
| BG065033 | Acads | 11409 | 1.51 | down | acyl-Coenzyme A dehydrogenase, |
| | | | | | short chain |
| BG083405 | Acat1 | 110446 | 1.52 | down | acetyl-CoA acetyltransferase |
| BG087153 | Bckdha | 12039 | 1.71 | down | branched chain ketoacid |
| | | | | | dehydrogenase E1, alpha |
| | | | | | polypeptide |
| BG073167 | Hsd17b4 | 15488 | 2.01 | up | hydroxysteroid (17-beta) |
| | | | | | dehydrogenase 4 |
| BG07999 | Echs1 | 93747 | 2.01 | down | enoyl Coenzyme A hydratase, short |
| | | | | | chain, 1, mitochondrial |
| BG087380 | Hadha | 97212 | 1.63 | down | hydroxyacyl-Coenzyme A |
| | | | | | dehydrogenase/3-ketoacyl- |
| | | | | | Coenzyme A thiolase/enoyl- |
| | | | | | Coenzyme A hydratase (trifunctional |
| | | | | | protein), alpha subunit |
| BG086728 | Hadhsc | 15107 | 2.33 | down | L-3-hydroxyacyl-Coenzyme A |
| | | | | | dehydrogenase, short chain |
| BG070984 | Ivd | 56357 | 1.79 | down | isovaleryl coenzyme A |
| | | | | | dehydrogenase |

Citrate cycle (TCA cycle)

| | | | | | |
|---|---|---|---|---|---|
| BG063733 | Cs | 12974 | 1.54 | down | citrate synthase |
| BQ550883 | Mdh2 | 17448 | 1.57 | down | malate dehydrogenase 2, NAD |
| | | | | | (mitochondrial) |
| BG067227 | Mdh1 | 14779 | 2.00 | down | malate dehydrogenase 1, NAD |
| | | | | | (soluble) |
| BG086026 | Sdha | 66945 | 1.86 | down | succinate dehydrogenase complex, |
| | | | | | subunit A, flavoprotein (Fp) |
| BG068897 | Sucla2 | 20916 | 2.17 | down | succinate-Coenzyme A ligase, ADP- |
| | | | | | forming, beta subunit |

Pyruvate metabolism

| | | | | | |
|---|---|---|---|---|---|
| BG083405 | Acat1 | 110446 | 1.52 | down | acetyl-CoA acetyltransferase |
| BG073920 | Ldh2 | 16832 | 1.56 | down | lactate dehydrogenase B |
| BQ550883 | Mdh2 | 17448 | 1.57 | down | malate dehydrogenase 2, NAD |
| | | | | | (mitochondrial) |
| BG067227 | Mdh1 | 17449 | 2.00 | down | malate dehydrogenase 1, NAD |
| | | | | | (soluble) |
| BG068736 | Pdha1 | 18597 | 1.58 | down | pyruvate dehydrogenase E1 alpha 1 |

Glycolysis/Gluconeogenesis

| | | | | | |
|---|---|---|---|---|---|
| BG075245 | Akr1a4 | 58810 | 1.54 | up | aldo-keto reductase family 1, |
| | | | | | member A4 (aldehyde reductase) |
| BQ554389 | Eno3 | 13808 | 2.90 | down | enolase 3, beta muscle |
| BG08251 | Fbp2 | 14120 | 1.54 | down | fructose bisphosphatase 2 |
| BG073920 | Ldh2 | 16832 | 1.56 | down | lactate dehydrogenase B |
| BG068736 | Pdha1 | 18597 | 1.58 | down | pyruvate dehydrogenase E1 alpha 1 |
| BG088948 | Pfkm | 18642 | 1.52 | down | phosphofructokinase, muscle |

Alanine and aspartate metabolism

| | | | | | |
|---|---|---|---|---|---|
| BG067706 | Gpt2 | 108682 | 1.69 | down | glutamic pyruvate transaminase |
| | | | | | (alanine aminotransferase) 2 |
| BG078765 | Got2 | 14719 | 1.66 | down | glutamate oxaloacetate transaminase |
| | | | | | 2, mitochondrial |
| BG064747 | Gpt1 | 76282 | 1.85 | down | glutamic pyruvic transaminase 1, |
| | | | | | soluble |
| BG079401 | Nars | 70223 | 1.51 | up | asparaginyl-tRNA synthetase |

TABLE 11
Comparison of gene expression measures by microarray and Northern blot
analyses. Data represents fold-change relative to hCryAB^{WT} controls.

| | Microarray | | Northern Blot | |
|---|---|---|---|---|
| | 3 months | 6 months | 3 months | 6 months |
| Ankrd1 | 6.64 | 8.35 | 5.74 | 20.67 |
| Cat | 2.26 | 2.16 | 4.16 | 4.49 |
| Gpx3 | 2.42 | N.S. | 2.51 | 4.82 |
| Hsp90aa1 | N.S. | 2.59 | 3.91 | 6.02 |
| Eno3 | 0.49 | 0.34 | 0.51 | 0.59 |
| Mdh1 | N.S. | N.S. | 0.59 | 0.59 |

N.S. = not significant or below 2-fold threshold by ANOVA (stringent) analysis. However, all such genes were identified as having significant changes at the threshold levels set for the pairwise analysis.

TABLE 12
Cellular pathways with altered expression in hR120GCryAB hearts
relative to hCryAB WT hearts.

| | | Genes | | | |
|---|---|---|---|---|---|
| | Genes | Identified | | z-score | |
| KEGG Pathway | on Array | Up | Down | Up | Down |
| 3 Month Up-regulated | | | | | |
| Glutathione metabolism | 18 | 5 | 0 | 6.44 | −0.49 |
| Antigen processing and | 24 | 5 | 2 | 5.38 | 3.02 |
| presentation | | | | | |
| Complement and coagulation | 25 | 4 | 0 | 4.02 | −0.58 |
| cascades | | | | | |
| 6 Month Up-regulated | | | | | |
| Antigen processing and | 24 | 6 | 1 | 5.92 | −0.05 |
| presentation | | | | | |
| Glutathione metabolism | 18 | 4 | 2 | 4.46 | 1.41 |
| Cell communication | 41 | 6 | 0 | 4.05 | −1.38 |
| Ribosome | 51 | 6 | 1 | 3.38 | −0.85 |
| Aminoacyl-tRNA biosynthesis | 23 | 3 | 0 | 2.59 | −1.03 |
| 6 Month Down-regulated | | | | | |
| Oxidative phosphorylation | 75 | 1 | 19 | −1.00 | 9.09 |
| Fatty acid metabolism | 26 | 1 | 9 | 0.14 | 7.61 |
| Valine, leucine and | 34 | 1 | 9 | −0.14 | 6.38 |
| isoleucine degradation | | | | | |
| Carbon fixation | 17 | 1 | 6 | 0.58 | 6.28 |
| Pyruvate metabolism | 22 | 0 | 5 | −0.88 | 4.25 |
| Citrate cycle (TCA cycle) | 24 | 0 | 5 | −0.92 | 3.98 |
| Glycolysis/Gluconeogenesis | 31 | 1 | 5 | −0.04 | 3.24 |
| Alanine and aspartate | 15 | 1 | 3 | 0.71 | 2.98 |
| metabolism | | | | | |

TABLE 13
Comparison of gene expression measures by microarray and Northern
blot analyses. Data represents fold-change in hR120GCryAB relative
to hCryAB WT controls. Corresponding fold-changes calculated
from the Affymetrix microarray validation are shown in parentheses.

| | cDNA Microarray | | | |
|---|---|---|---|---|
| | Fold-Change | | | |
| | (Affymetrix Microarray | | Northern | |
| | at 6 months) | | Blot Fold-Change | |
| | 3 months | 6 months | 3 months | 6 months |
| Ankrd1 | 6.64 | 8.35 (6.34) | 5.74 | 20.67 |
| Cat | 2.26 | 2.16 (4.20) | 4.16 | 4.49 |
| Gpx3 | 2.42 | N.S. (3.47) | 2.51 | 4.82 |
| Hsp90aa1 | N.S. | 2.59 (4.20) | 3.91 | 6.02 |
| Eno3 | 0.49 | 0.34 (0.41) | 0.51 | 0.59 |
| Mdh1 | N.S. | N.S. (N.S.) | 0.59 | 0.59 |

N.S. = not significant by ANOVA analysis or below 2-fold threshold. However, all such genes were identified as having significant changes at the threshold levels set for the pairwise analysis.

### 3. Example 3

### Human αB-Crystallin Mutation Causes Oxido-Reductive Stress and Protein Aggregation Cardiomyopathy in Mice

i. Results

Transgene Overexpression of WT and Human R120GCryAB in Mice:

To create a small animal model of missense human R120GCryAB expression (hR120GCryAB), transgenic mice were generated using the mouse c-myosin heavy chain (cMHC) promoter driving the expression of either the human cDNA CryAB wild-type (hCryAB Tg) gene or the R120G mutated form in a tissue-specific manner. Two transgenic lines were established for each construct; lines 3241 and 3244 for cMHC hCryAB Tg and lines 7302 and 7313 for cMHC hR120GCryAB Tg. Transgene transmission to the off-spring was analyzed by Southern blot and PCR. CryAB protein in both supernatant and pellet fractions of heart homogenates from 6 month old mice was probed by Western blot for nontransgenic controls (NTg), hCryAB Tg, and hR120GCryAB Tg animals (FIG. 1A). Total CryAB protein, reflecting endogenous and transgene expression, was increased 1.5 fold greater in line 3241 hCryAB Tg, 2 fold in line 7313 hR120GCryAB Tg and 6 fold in line 7302 hR120GCryAB Tg (FIG. 1A and FIG. 1B). These two transgenic lines, with mild and moderate hR120G CryAB overexpression, were designated hR120GCryAB Low Tg and hR120GCryAB High Tg, respectively. Whereas hCryAB Tg protein remained entirely soluble, hR120GCryAB Tg protein was found in both soluble and insoluble fractions, indicating that mutant protein expression recapitulates the protein aggregation disorder, a proposed model for desmin-related myopathies (Vicart et al., 1998; Wang et al., 2001).

Cardiac-Specific hR120GCryAB Overexpression Causes Lethal Cardiomyopathy With Variable Penetrance:

Moderate overexpression of hR120GCryAB Tg protein in the mouse heart induced cardiac hypertrophy, progressive heart failure and premature death (FIG. 2A, FIG. 2E). Magnetic resonance imaging (MRI) was used to confirm cardiac hypertrophy and severe ventricular remodeling with dilatation in end-stage hR120GCryAB Tg cardiomyopathic mice (Table 4). At 6 months, morphological analyses consistently revealed gross four-chamber enlargement, biatrial thrombosis and cardiac hypertrophy in hR120GCryAB High Tg mice (FIG. 2A and Table 5). Large aggregates were present in myocardial sections of hR120GCryAB High Tg but were not present in either hCryAB Tg or hR120GCryAB Low Tg mice.

Beyond 6 months, the rate of disease progression accelerated for hR120GCryAB High Tg animals characterized by increased lethargy and systemic edema from fluid retention, reaching 100% mortality at 66 weeks (FIG. 2E). Consistent with this accelerated attrition, the viability of cardiomyocytes isolated from hR120GCryAB High Tg was significantly decreased compared with either hR1 20GCryAB Low Tg or NTg control hearts (FIG. 3A). A 20% mortality from sudden death after 80 weeks was noted in hR120GCryAB Low Tg mice (FIG. 2E). There were no effects on mortality in either hCryAB Tg mice or nontransgenic (NTg) littermates over 80 weeks (FIG. 2B). Neither abnormal baseline cardiac function nor overt signs of heart failure were present in hR120GCryAB LowTg mice (Table 5), but cardiac contractile reserve in response to dobutamine challenge was decreased compared with NTg controls (FIG. 3D).

RNA dot blots showed that markers of cardiac hypertrophy and congestive heart failure, such as atrial natriuretic factor (ANF) and brain natriuretic factor (BNF), were all increased at 3 and 6 months, whereas phospholam ban (PLN) expression, a major regulator of cardiac contractility and relaxation, was decreased with the onset of heart failure in 6-month old hR120GCryAB High Tg myopathic hearts (FIG. 2D).

Major Hsps, Especially Hsp25, are Induced by hR1 20GCryAB Expression:

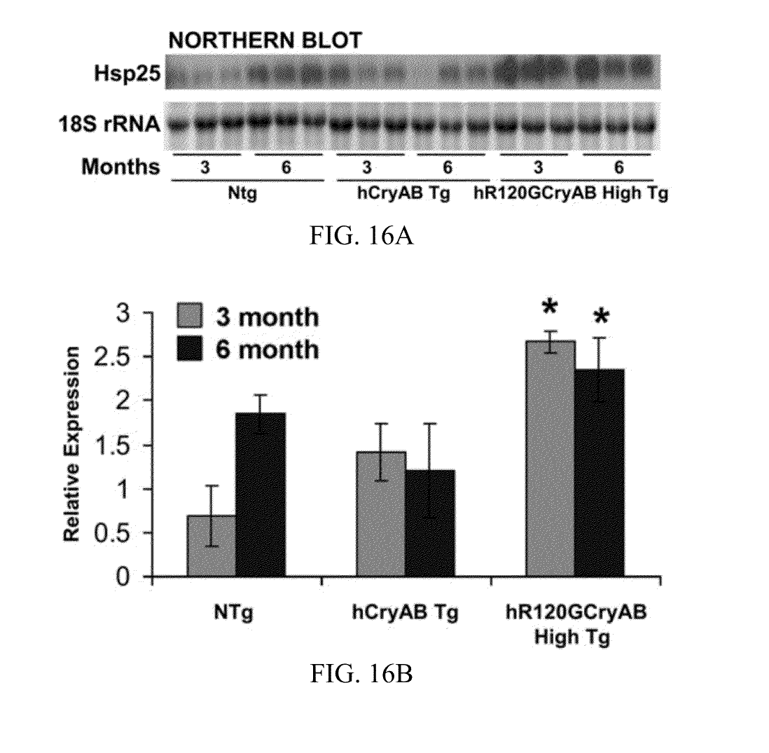

Activation of stress response pathways exemplified by members of the multigene families of heat shock proteins (Hsps) has been documented in human heart failure (Knowlton et al., 1998). To characterize the effects of hR120GCryAB overexpression on Hsp expression in myopathic hearts, representative members of the major Hsp families were assessed by Western blot analysis in 6 month old mice, an arbitrary transition point associated with progression of heart failure and increased mortality. Levels of Hsp90, an ATP-dependent chaperone that forms multiprotein complexes, were 2 fold higher for hR120GCryAB High Tg hearts compared to NTg, hCryAB Tg, or hR120GCryAB Low Tg hearts in both soluble and insoluble fractions (FIG. 4A-FIG. 4D). Similarly, Hsp70 levels were increased by 2 fold in the soluble fraction of cardiac homogenates of hR120GCryAB High Tg compared with NTg expression. Hsp25 protein, a non-ATP dependent chaperone that forms multimeric oligomers, was modestly increased in the supernatant fraction, but this chaperone was >25 fold higher in the insoluble fraction of hR120GCryAB High Tg hearts compared to NTg, hCryAB Tg, or hR120GCryAB Low Tg hearts (FIG. 4B and FIG. 4D). Of note, levels of Hsp25 were indistinguishable among these four experimental groups at 2 months (FIG. 4E-FIG. 4F) but mRNA levels of Hsp25 were increased by 2.5 fold in hR120GCryAB High Tg compared with hCryAB Tg at 3 and 6 months (FIG. 16A and FIG. 16B), indicating hR120GCryAB Tg protein expression causes upregulation of stress-inducible Hsps in vivo. These data indicate that hR120GCryAB Tg protein expression causes differential upregulation of stress-inducible Hsps in vivo with a major effect on Hsp25 expression.

R120GCryAB Expression Causes Early Enhancement of Antioxidative Pathways:

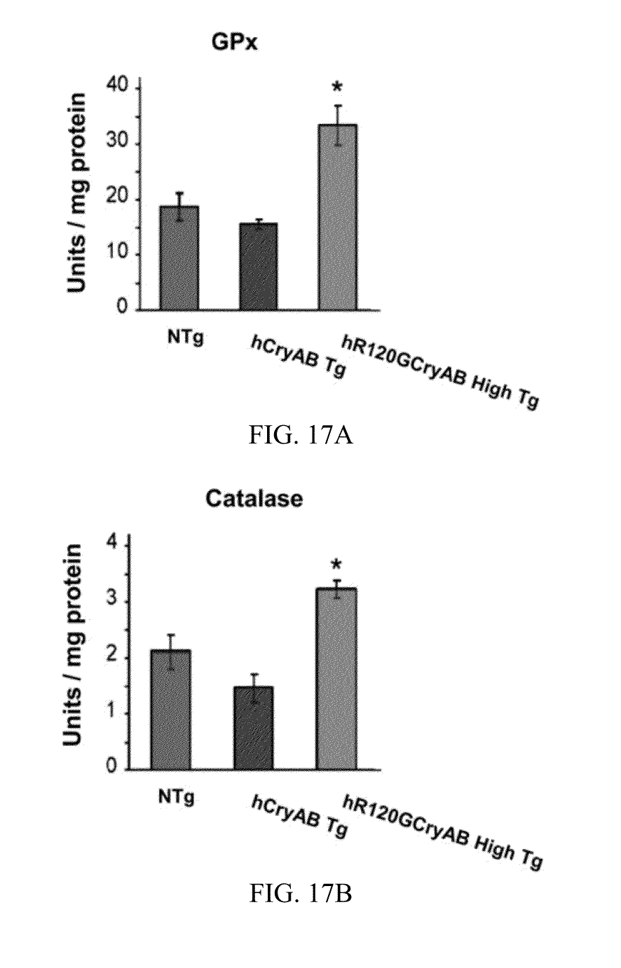

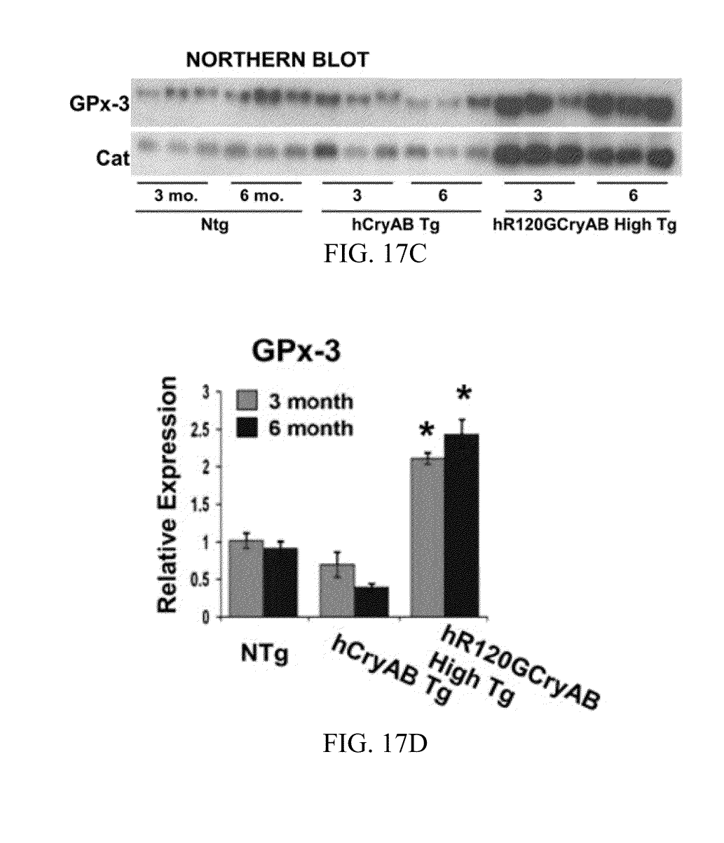

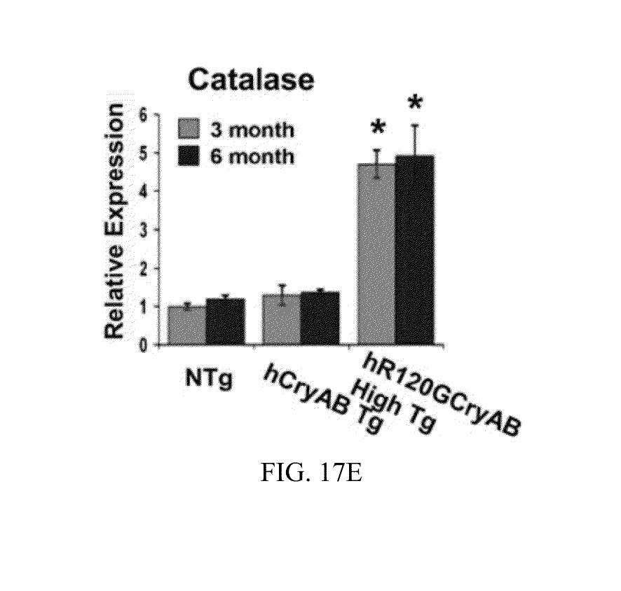

It was next determined if increased synthesis of major Hsps are accompanied by the induction of antioxidant pathways, which detoxify ROS in vivo. Both catalase and the glutathione peroxidase catalyze the disposition of H₂O₂ into H₂O and O₂. The enzymatic activity of glutathione peroxidase, which catalyzes the elimination of peroxides, was 70% higher in hR120GCryAB High Tg hearts compared with NTg controls at 6 months (FIG. 17A), but cytosolic GPx-1 protein assessed by immunoblot analysis was similar among all groups (FIG. 7D and FIG. 7E). Moderate increase in GPx activity (FIG. 17A) without a commensurate increase in GPx protein expression can reflect the translational limitations of available selenium, which is not standardized in chows, and/or of the translational cofactors required for selenoprotein synthesis (Handy et al., 2006). Similarly, the activity of catalase in hR120GCryAB High Tg was 50% and 100% higher than either NTg or hCryAB Tg hearts, respectively (FIG. 17B) and protein abundance of catalase in hR120GCryAB High Tg was 5- and >2-fold greater than either NTg or hCryAB Tg hearts, respectively (FIG. 7D and FIG. 7E).

At both 3 and 6 months, it was observed that mRNA levels of glutathione peroxidase (GPx-3) and catalase were 2.5 and 5 fold higher in hR120GCryAB High Tg hearts compared with NTg controls, respectively (FIG. 17C-FIG. 17E). As upregulation of HSP stress pathway parallel the activation of antioxidative enzymes at 3 and 6 months (FIG. 16A and FIG. 16B and FIG. 17C-FIG. 17E), the results indicate that key cytoprotective pathways are recruited as early compensatory events in response to mutant hR120GCryAB expression, in part, to mitigate increased oxidative stress.

TABLE 14
Concentrations of Reduced (GSH) and Oxidized (GSSG) Glutathione in Heart Tissue
Homogenates at 6 Months

| | | | hR120GCryAB | hR120GCryAB |
|---|---|---|---|---|
| Parameter/Groups | Nontransgenic | hCryAB Tg | Low Tg | High Tg |
| Total GSH | 811.19 ± 125.87 | 937.06 ± 97.90 | 1006.01 ± 58.74 | 1573.02 ± 33.57† |
| (nmol/mg) (N = 6) | | | | |
| GSSG | 18.20 ± 1.6** | 24.51 ± 1.7 | 24.01 ± 0.8 | 24.51 ± 0.9 |
| (nmol/mg) (N = 6) | | | | |
| GSH/GSSG | 44.39 ± 3.02 | 38.17 ± 1.35 | 41.88 ± 1.05 | 64.54 ± 3.50* |

Values are expressed as mean ± SD calculated for six animals in each individual experiment.
†p = 0.001 hR120GCryAB High Tg compared to other groups.
**p < 0.025 NTg compared to other groups.
*p < 0.05 hR120GCryAB High Tg compared to other groups.

R120GCryAB Tg Expression Causes Oxido-Redox Shift Toward Reductive Stress:

It was next determined if myopathic hearts might respond with increased GSH levels and alterations in redox balance as Hsp25 has been implicated in GSH metabolism (Baek et al., 2000; Mehlen et al., 1996). The concentrations of reduced glutathione (GSH) and oxidized glutathione (GSSG) in heart homogenates of 6 month old experimental groups are shown in Table 14. The relative amounts of total GSH revealed the following rank order: hR120GCryAB High Tg>hR120GCryAB Low Tg>hCryAB Tg>NonTg. The total GSH content of hR120GCryAB High Tg was significantly increased by 2 fold compared with NTg controls (Table 14). The amount of GSSG in all Tg groups was 25% higher than NTg controls, but only the higher GSH:GSSG ratio in hR120GCryAB High Tg hearts reached statistical significance compared to NTg controls.

The susceptibility of intracellular lipids to peroxidation was next assessed using malondialdehyde (MDA) and proteins to undergo oxidative modifications by antidinitrophenylhydrazine (DNPH) immunostaining as surrogate biomarkers (FIG. 5A-FIG. 5C). At 6 months, both MDA levels and anti-DNPH immunoreactive proteins were significantly lower in hR120GCryAB High Tg hearts compared with the NTg control (FIG. 5A, FIG. 5B, respectively). Taken together, these results indicate that the effects of high level of hR120GCryAB expression dramatically increases reducing power, exemplified by the higher GSH concentrations and GSH:GSSG ratio.

R120GCryAB Overexpression Activates the GSH Biosynthesis-Recycling Pathway:

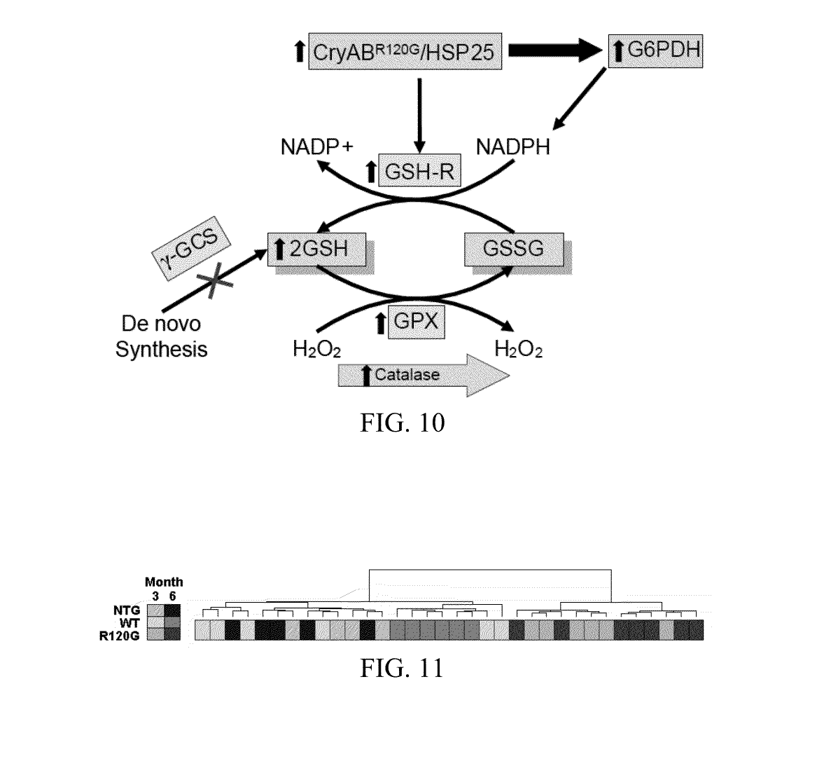

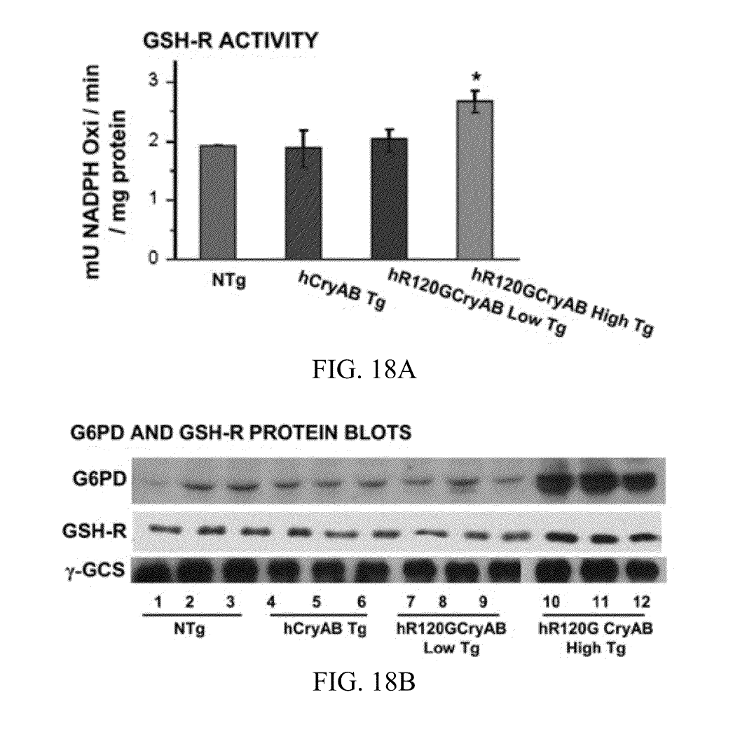

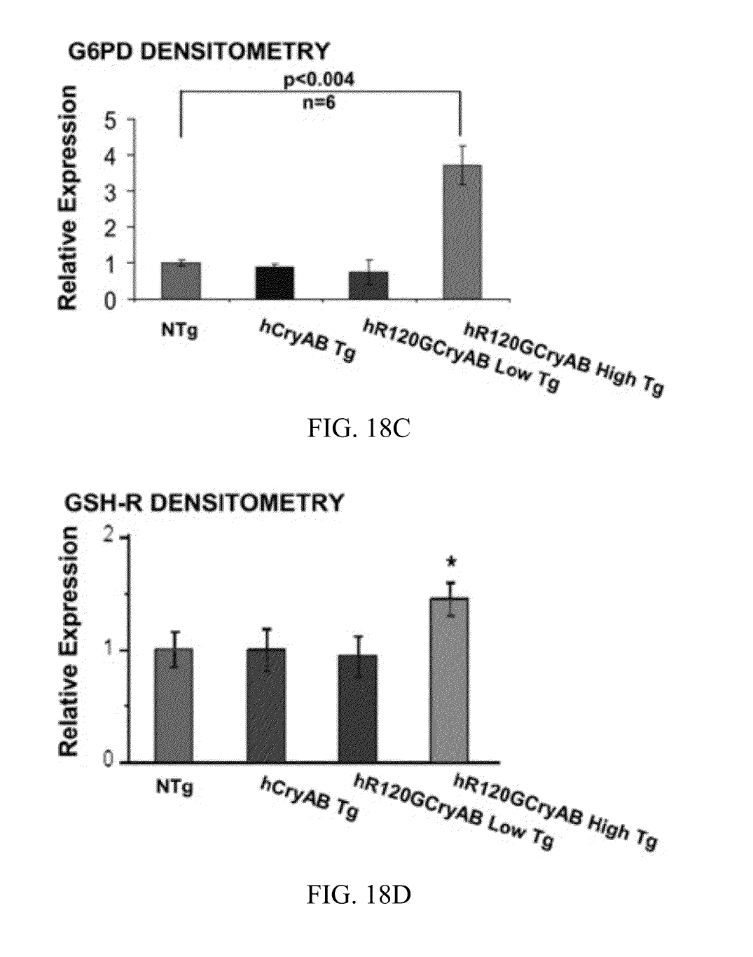

The findings of increased expression and activities of key antioxidative enzymes such as catalase and glutathione peroxidase, and GSH elevation (Table 14), in hR120GCryAB High Tg heart homogenates warranted a systematic assessment of each enzymatic step that catalyzes either the recycling of GSH and/or de novo synthesis pathways (FIG. 10). Reduced glutathione (GSH) is generated from oxidized GSSG by the oxidation of nicotinamide adenine-dinucleotide phosphate, NADPH, a product of the glucose-6-phosphate dehydrogenase (G6PD) reaction. G6PD is the rate-limiting enzyme of the pentose phosphate “shunt” pathway of anaerobic glycolysis (Preville et al., 1999). The G6PD enzyme activity in heart homogenates for hR120GCryAB High Tg was 2 fold greater than NTg, hCryAB Tg, or hR120GCryAB Low Tg at 6 months (FIG. 6A). Myocardial abundance of G6PD protein, however, was 4 fold higher in hR120GCryAB High Tg than NTg, hCryAB Tg, or hR120GCryAB Low Tg at 6 months (FIG. 18B and FIG. 18C).

Glutathione reductase (GSH-R) activity was next tested, which uses NADPH as the principal source of reducing equivalents for recycling oxidized GSSG to reduced GSH. Both enzymatic activity and protein content of GSH-R were significantly increased in hR120GCryAB High Tg hearts compared to NTg, hCryAB Tg, and hR120GCryAB Low Tg hearts at 6 months, (FIG. 18A, FIG. 18B, and FIG. 18D). The enzymatic activity and protein abundance of gamma-glutamyl cysteine synthetase (g-GCS), the rate-limiting enzyme for biosynthesis under feedback inhibition by GSH, were indistinguishable among all experimental groups examined, indicating that increased GSH recycling pathway, and not de novo biosynthesis, is the predominant mechanism responsible for elevated GSH levels in response to increased hR120GCryAB expression.

Cardiac-Specific hR120GCryAB Promotes Interactions with Hsp25 and G6PD:

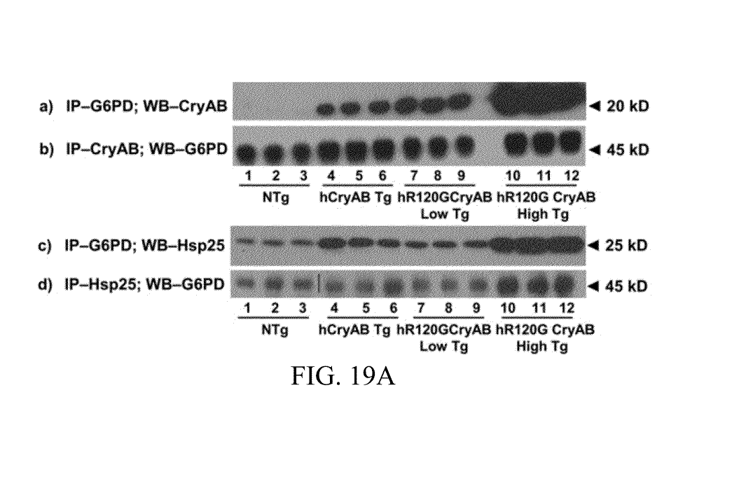

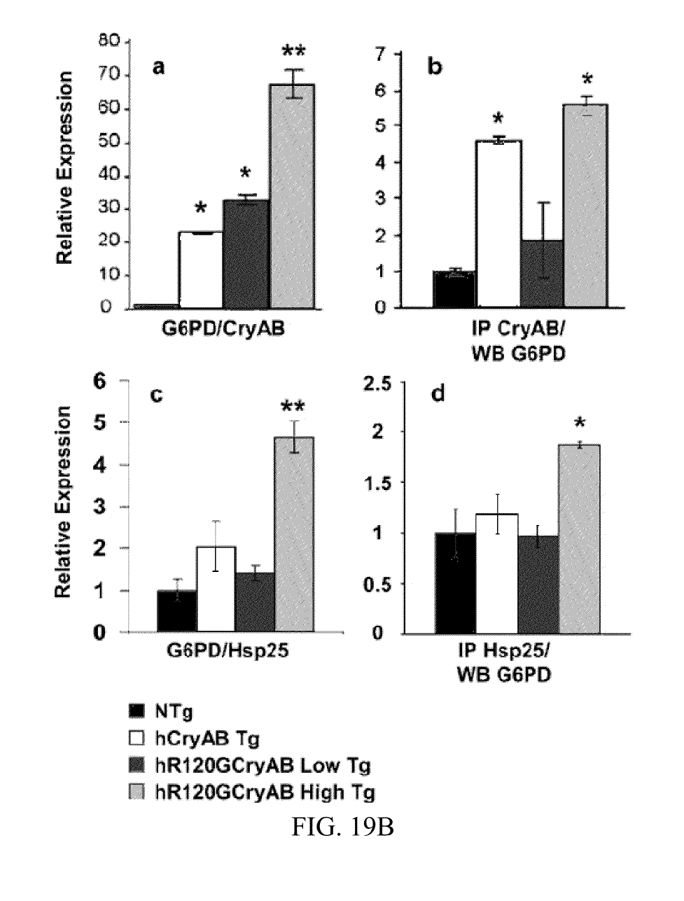

Vulnerability to hR120GCryAB expression can arise from a toxic gain-of-function mechanism caused by other client protein interactions with either Hsp25 and/or G6PD. To determine if hR120GCryAB protein expression has direct effects on molecular interactions involving the GSH biosynthetic pathway, reciprocal coimmunoprecipitations and immunoblot analysis were performed in heart homogenates. The interactions between G6PD and either CryAB or Hsp25 were found in heart extracts from hCryAB Tg, hR120GCryAB Low Tg and hR120GCryAB High Tg but were negligible for NTg (FIG. 19A). More robust molecular interactions were seen for both CryAB and Hsp25 for G6PD, which can represent chaperone-dependent properties in vivo (FIG. 19A and FIG. 19B).

Using confocal microscopy, the patterns of distribution and localization for Hsp25 and CryAB were similar within the core of large protein aggregates. In contrast, G6PD was more diffusively distributed along the myocardial striations and occasionally but not exclusively surrounding protein aggregates containing both CryAB and Hsp25 proteins. These findings provide evidence that molecular interactions between mutant CryAB and Hsp25 or G6PD can promote the pathogenesis of hR120GCryAB expression leading to cardiomyopathy.

G6PD Deficiency Prevents Cardiac Hypertrophy and Protein Aggregation in hR120GCryAB High Tg Cardiomyopathic Mice:

If causal mechanisms are linked to marked upregulation of G6PD, then maneuvers that either inhibit and/or down-regulate this pathway should reverse redox imbalance triggering hR120GCryAB Tg cardiomyopathy. Thus, male hemizygous G6PD mutant mice (G6PD^{mut}, C3H background) were crossed with heterozygote hR120GCryAB High Tg animals to generate hR120GCryAB High Tg/G6PD^{mut} mice. In the G6PD^{mut} homogenates, the X-linked gene encoding G6PD maintains 20% of the normal enzymatic activity under the control of the native promoter (FIG. 9A).

G6PD enzyme activity and expression in hR120GCryAB High Tg were 2.5-3.0 fold greater than in either NTg or hR120GCryAB High Tg/G6PD^{mut} (FIG. 9A-FIG. 9D). In contrast, the modulation of G6PD enzyme activity and expression in hR120GCryAB High Tg/G6PD^{mut} hearts was not different from NTg. Compared with NTg animals, GSH content was modestly increased in hR120GCryAB High Tg (30%) and hR120GCryAB High Tg/G6PD^{mut} (14%).

Moreover, the increases in total CryAB and Hsp25 protein levels were similar between hR120GCryAB High Tg and hR120GCryAB High Tg/G6PD^{mut} hearts, indicating myocardial total CryAB or Hsp25 expression induced by the hR120GCryAB High transgene was unaltered by G6PD deficiency in vivo.

Cardiac hypertrophy is a constant finding of hR120GCryAB High Tg cardiomyopathy and a major risk for heart failure in experimental models and humans alike. Indeed, heart weight/body weight ratio in 6 month old hR120GCryAB High Tg was 33% greater than hR120GCryAB High Tg/G6PD^{mut} (6.15±1.06 versus 4.63±0.27, p<0.05), the latter being similar to NTg (4.63±0.27 versus 4.50±0.19, NS) as shown in FIG. 9B. Such profound effects in preventing the hypertrophic response in hR120GCryAB High Tg/G6PD^{mut} hearts were confirmed at the molecular level using several biomarkers for cardiac hypertrophy (FIG. 9E, panel (i) shows BNF, panel (ii) shows PLN, and panel (iii) shows SERCA-2A). Lastly, G6PD^{mut} intercross with hR120GCryAB High Tg completely prevents protein aggregation (FIG. 9E), consistent with abrogating the manifestations of cardiomyopathy.

Of note, the decreased survival of cardiomyocytes from 6 month old hR120GCryAB High Tg, which was reduced by 30% compared with age-matched hR120GCryAB Low Tg or NTg animals (FIG. 3A), was fully reversed by G6PD deficiency. The reversal in G6PD enzyme activity, prevention of protein aggregation, and abrogation of cardiac hypertrophy in hR120GCryAB High Tg/G6PD^{mut} hearts demonstrate for the first time that G6PD plays a key role in production of reductive stress of the disease-causing hR120GCryAB mutation in mammals.

MRI Studies of R120GCryAB Cardiomyopathy in Mice:

Magnetic imaging resonance (MRI) was used to assess the effects of hR120GCryAB expression on cardiac function in vivo. Serial measurements of ventricular cavity dimension, left ventricular mass (LVM) and left ventricular ejection fraction (LVEF) were made at 3, 6 and 10 months (Table 5). The hCryAB Tg mouse line, with mild wild-type CryAB overexpression, was selected as a control. At both 3 and 6 months, no differences in cavity dimension and cardiac function were observed in hCryAB Tg, hR120GCryAB Low Tg and hR120GCryAB High Tg animals (Table 5). There was a trend towards greater LV mass in hR120GCryAB Tg High mice compared with either hR120GCryAB Low Tg or hCryAB Tg at 6 months (Table 5). Cardiac hypertrophy was most pronounced in hR120GCryAB High Tg mice at 10 months compared to hR120GCryAB Low Tg and/or hCryAB Tg (Table 5). Likewise, left ventricular ejection fraction was decreased at 10 months for hR120GCryAB High Tg compared with either hR120GCryAB Low Tg or hCryAB Tg animals (Table 5). These results indicate that cardiac hypertrophy and severe ventricular remodeling with dilatation are specific hallmarks of end-stage hR120GCryAB Tg protein aggregation cardiomyopathy in mice.

R120GCryAB Low Tg Mice Devoid of Large Aggregates Exhibit Decreased Cardiac Contractile Reserve:

To assess the effects of hR120GCryAB Tg on cardiac myocyte viability, isolated left ventricular myocytes were cultured as described in FIG. 3A (Boston et al., 1998). The survival of cardiomyocytes from age-matched, 6 month old hR120GCryAB High Tg was reduced by 30% compared with either hR120GCryAB Low Tg or NTg animals (FIG. 3A). Both myocyte viability and cardiac function of hR120GCryAB Low Tg were normal between 12 and 40 weeks under basal conditions (Table 5), but mortality at 80 weeks was increased by 20 percent (FIG. 2E). To determine if subtle cardiac abnormalities could be detected in hR120GCryAB Low Tg mice, experimental groups were subjected to 300 nM dobutamine challenge, an established method to assess cardiac reserve (Grupp et al., 1993). In the isolated perfused Langendorff heart, myocardial external work and maximal rates of contraction before, during, and after exposure to dobutamine revealed a myopathic effect of even mild hR120GCryAB overexpression compared to NTg (FIG. 3D).

Biomarkers of Oxidative Stress are Altered by hR120GCryAB Expression:

Reactive oxygen species (ROS) have been implicated in the pathogenesis of cardiac hypertrophy and heart failure (Giordano, 2005; Griendling and FitzGerald, 2003; Yamamoto et al., 2003). The susceptibility of intracellular lipids and proteins to oxidative modifications as surrogate biomarkers was assessed. Lipid peroxidation was measured using malondialdehyde (MDA) as a biomarker of oxidative stress (FIG. 5A). MDA was significantly lower (by 40%) in hR120GCryAB High Tg hearts at 6 months compared with the NTg control (FIG. 5A). To corroborate these age-dependent effects, myocardial levels of protein carbonyl content were assessed by anti-dinitrophenylhydrazine (DNPH) immunostaining for specific amino acid residues modified by reactive oxygen species (Stadtman, 1992). At 3 and 6 months, tissue levels of anti-DNPH immunoreactive proteins were also elevated in both hCryAB Tg and hR120GCryAB Low Tg hearts compared with the NTg control (FIG. 5B-FIG. 5C). In contrast, there was profound lowering of protein carbonyl levels in hR120GCryAB High Tg hearts between 3 and 6 months (FIG. 5B-FIG. 5C). The unexpected lowering in the carbonyl content in hR120GCryAB High Tg hearts at 6 months is consistent with either an exaggerated increase in antioxidant enzymes or marked enhancement in the reducing equivalents, or both.

ii. Methods

Transgenic Constructs, Mouse Lines, and Care:

The full-length human B-crystallin (CryAB) was obtained. The missense mutation, R120G, was created from the human CryAB cDNA by PCR-based mutagenesis (Quick Change Site directed mutagenesis kit, Stratagene, LaJolla) and confirmed by sequencing. Subsequently, the cDNAs were placed under the control of alpha-myosin heavy chain (MHC) promoter. Transgenic mice were generated by pronuclear injection according to standard procedure. Founders were identified by PCR and Southern blot analysis and crossed with wild-type C57/BL6 mice to establish the trans-genic lines. Hemizygous mice for the X-linked gene encoding G6PD with 20% of the normal enzymatic activity were obtained (Leopold et al., 2003). Standard mouse breeding was used to generate compound R120G High/G6PD^{mut} heterozygotes. Mice were fed with standard diet and had access to water and food ad libidum; they were housed under controlled environment with 23±2° C. and 12 hr light/dark cycles.

Antibodies and Reagents:

The following antibodies and reagents were used: an anti-CryAB polyclonal antibody, which recognizes both the mouse and human proteins, was raised against residues 164-175 of human CryAB. Rabbit anti-Hsp25, anti-Hsp70, anti-Hsp90 (StressGen, Victoria, BC, Canada) and rabbit anti-G6PD (Novus Bio.), anti-catalase, anti-glutathione peroxidase, anti-glutathione reductase (AbCam), gamma-GCS/glutamate cysteine ligase-Ab 1 (Labvision, Neomarkers, CA) and Anti-DNP (Sigma Chemicals Co, St. Louis, Mo.) antibodies were purchased from commercial vendors. Acrylamide/bis-acrylamide, ammonium persulfate, protein assay reagent, protein standard markers (Bio-Rad, Richmond, Calif.) and enzymatic assay kits for reduced and oxidized glutathione, catalase, glutathione peroxidase, glutathione reductase were obtained from Bioxitech (Oxis Research). RNeasy, DNA purification kits (QIAGEN, Valenica, CA) and Northern Max kit (Ambion, Austin, Tex.), α[³²P] dATP (Amersham) were obtained commercially.

Glutathione Measurements:

Hearts were dissected, atria and large vessels trimmed and rinsed briefly in PBS. Hearts were weighed, flash frozen, pulverized and homogenized in 5% sulphosalisilic acid (SSA) and centrifuged, 10,000×g, at 4° C. for 10 min. Supernatant was removed and used for GSH assay. GSSG content was measured by using 100 μl fraction of the supernatant adding 2 μl of 2-vinylpyridine and 10 μl of 50% triethanolamine, which was kept at room temperature for 1 hr. Total glutathione and oxidized glutathione (samples derivatized with 2-vinyl pyridine) were measured by a standard recycling assay based on the reduction of 5,5-dithiobis-2-nitrobenzoic acid in the presence of glutathione reductase and NADPH (Griffith, 1980).

Glucose-6-Phosphate Dehydrogenase Activity:

Cytoplasmic extracts were prepared as described above and were used to assess the G6PD activity (Hochman et al., 1982). Protein aliquots were prepared in 90 μM triethanolamine, (pH 7.6), 10 mM MgCl₂, 198 μM G-6-phosphogluconate and 100 μM NADP+. Similar reaction mixtures with 198 μM of glucose-6-phosphate were also prepared to measure the activity of 6-phospho gluconate dehydrogenase. The solutions were mixed and absorbance was read at 340 nm every 2 min for 20 min. The specific activity of glucose-6-phosphate dehydrogenase was determined by calculating the difference between the readings from the two reactions.

Morphological Analysis and Immunohistofluorescence Assays:

The right atrium of anesthetized mice was cut and the hearts were perfused through the apex with saline (0.9% NaCl) for 5 min to remove all blood. Hearts were then fixed for 10-12 min by perfusion of 0.1% paraformaldehyde in cardioplegic buffer (50 mM KCl and 5% dextrose), removed from the chest cavity, cut in half coronally, and cryoprotected by successive incubations in 10% sucrose in PBS/0.05% NaN₃ (at least 3 hr) and 30% sucrose in PBS/0.05% NaN₃ (at least 6 hr). Hearts were frozen in OCT and sectioned at 5 μm using a cryostat. Dried cryosections were washed in PBS and blocked for 30 min at room temperature in blocking solution (1% BSA, 0.1% fish skin gelatin [Sigma G7765], 0.1% Tween 20, and 0.05% NaN₃ in PBS). For detecting CryAB alone, the sections were incubated at room temperature for 45 min with rabbit anti-CryAB antibody (1:100 in PBS), washed three times with PBS for 5 min each wash, and then incubated at room temperature for 45 min with donkey anti-rabbit Alexa 488 (1:100; Molecular probes A21206) and TO-PRO-3 642/661 (1:100 of 1 mM stock solution dissolved in DMSO; Molecular Probes T3605). The sections were washed three times as described above and stained with phalloidin-Alexa 568 (1/20 dilution in PBS of a stock solution containing 0.2 units/μl dissolved in methanol; Molecular Probes A12380) at room temperature for 20 min, washed three times as described above, and then mounted with Vectashield Hard Set mounting medium (Vector Laboratories). Nail polish was used to seal the edges of the cover-slip once the mounting medium dried. The sections were observed and photographed using a laser-scanning Olympus IX81 confocal microscope equipped with Argon and HeNe excitation lasers at 488 nm, 543 nm, and 633 nm.

When performing immunohistofluorescence assays to detect CryAB, Hsp25, and G6PD simultaneously, the following modified procedure was followed. The rabbit anti-G6PD antibody (1:50 in PBS; Novus NB1 00-236) was first incubated with the tissue sections as described above and excess unbound antibody was removed by three washes in PBS for 5 min each. Bound rabbit anti-G6PD was then converted to goat antibody by incubating the tissues with goat anti-rabbit Fab (1:20 dilution in PBS; Jackson Immuno-Research 111-007-003) at room temperature for 45 min. The tissues were washed three times with PBS as described above and then incubated 45 min at room temperature with donkey anti-goat Alexa 488 (1:100 in PBS; Molecular Probes A1 1055) to detect G6PD and donkey anti-mouse Fab antibody (1:20 in PBS; Jackson Immuno-Research 715-007-003) to block endogenous mouse immunoglobulins in preparation for the anti-Hsp25 antibody. A control slide was incubated with goat anti-rabbit Alexa 633 (1:100 in PBS; Molecular Probes A21 070) to ensure complete conversion of the rabbit antibody to an immunoreactive goat antibody. The tissue sections were washed three times and incubated for 45 min at room temperature with rabbit anti-CryAB (1:100 in PBS; made at University of Texas Southwestern) and a mouse monoclonal anti-Hsp25 antibody (1:25 in PBS; Sigma H-0273). Following three washes, the tissue sections were incubated for 45 min at room temperature with the following secondary antibodies each at a 1:100 dilution: 1) goat anti-rabbit Alexa 633 (see above) to detect CryAB and 2) donkey anti-mouse Alexa 555 (Molecular Probes A31 570) to detect Hsp25. The slides were washed three times in PBS and mounted, observed, and photographed as described herein.

Statistics:

Statistics were performed using independent sample t tests, with the p values adjusted for six pair-wise comparisons using Finner's multiple comparison procedure (Finner, 1993). Data were expressed as mean±SD for >6 mice in each group. p<0.05 was considered significant.

Protein Isolation and Western Blot:

Hearts were harvested from animals and flash frozen in liquid nitrogen. Tissue was pulverized and homogenized in 25 mM HEPES, pH 7.4, 4 mM EDTA, 1.0 mM PMSF and Roche complete protease inhibitor cocktail. The extract was then centrifuged at 8,000 g for 30 minutes at 4° C. The pellet was then resuspended in 20 mM Tris, pH 6.8, 1.0 mM EDTA and 1.0% SDS and briefly sonicated into solution. Protein concentrations for supernatant and pellet were determined using Bio-Rad protein assay kit. Equal amounts of protein extracts (10-20 μg) were loaded and separated by SDS-PAGE. Proteins were then transferred electrophoretically from the gels to Immobilon-P (Millipore) membrane. Blots were blocked in Tris Buffered Saline-Tween 20 (TBST) containing 5% (w/v) dry milk followed by incubation for 2 hrs with the respective primary antibody diluted in TBS buffer. Blots were then washed three times for 10 min each in TBST and incubated with anti-rabbit (1:25000) or anti-mouse (1:10000) IgG horseradish peroxidase (Vector Labs) conjugated secondary antibody in TBS for 1 hr. After washing 5 times for 10 min each in TBS, the membranes were treated with ECL detection reagents (Pierce, Amersham Bio) and the proteins were visualized by exposure to Blue sensitive biofilm (Hyblot Autoradiography, Denville Scientific, Inc.).

Immunoprecipitation and Immunoblotting:

Heart homogenates were first prepared in TBS (10 mM Tris, 150 mM NaCl, pH 7.4) and centrifuged at 10,000 g for 15 minutes at 4° C. The cytosol was used to immunoprecipitate CryAB, Hsp25 and G6PD proteins. About 100 μg of protein sample was incubated with the respective antibody and gently rotated for 12-14 hrs, at 4° C. 25 μl of protein-A sepharose beads were added to the antigen-antibody complex and continued the incubation for 3 hrs. The antigen-antibodybeads complex was then washed 5 times with IP buffer (HEPES −10 mM, pH 7.4, NaCl—50 mM, glycerol—10%, DTT—1 mM, and standard protease inhibitors). The final precipitate was diluted with 2× gel loading buffer and precipitated proteins were resolved in 10 or 12% PAGE and immunoblotted using respective antibodies. Similarly, reciprocal IP was performed to reveal the protein interactions.

Dissociation of Adult Mouse Ventricular Myocytes:

Adult mouse myocyte isolation was performed with a modification of a previously described technique (Kadono et al., 2006). Briefly, hearts were removed from anesthetized mice and immediately attached to an aortic cannula. After perfusion with Ca²⁺-free modified Tyrode's solution for 5 minutes, hearts were digested with 0.25 mg/mL liberase Blendzyme 1 (Roche Molecular Biochemicals) in 25 mmol/L CaCl₂-containing modified Tyrode's solution for 6-8 minutes. The solution consisted of (mmol/L) NaCl 126, KCl 4.4, MgCl₂ 1.0, NaHCO₃ 18, glucose 11, HEPES 4, and 0.13 U/mL insulin and was gassed with 5% CO₂/95% O₂, which maintained the pH at 7.4. The digested hearts were removed from the cannula, and the left ventricles were cut into small pieces in 100 μmol/L Ca²⁺ containing modified Tyrode's solution. These pieces were gently agitated and then incubated in the same solution containing 2% albumin at 30° C. for 20 minutes. The cells were allowed to settle down with gravity. The supernatant was completely removed with a pipette and myocytes resuspended in 200 μmol/L Ca²⁺ and 2% albumin Tyrode's solution and allowed to settle for 20 minutes at 30° C. The cells were then resuspended in culture medium composed of 5% heat-inactivated fetal bovine serum (Hyclone), 47.5% MEM (GIBCO Laboratories), 47.5% modified Tyrode's solution, 10 mmol/L pyruvic acid, 4.0 mmol/L HEPES, and an additional 6.1 mmol/L glucose at 30° C. in a 5% CO₂ atmosphere. The percentage of normal rod-shape myocytes was determined by phase contrast microscopy after 1 hour incubation in culture medium at 30° C. in a 5% CO₂ atmosphere and was taken as an index of viability (Boston et al., 1998).

Isolated Heart Perfusion Studies:

Mice were anesthetized with an intraperitoneal injection of 50 mg/Kg body weight of sodium pentobarbital. Hearts were weighed and myocardial function was evaluated at 37° C. using an isolated Langendorff heart preparation as previously described (Neely et al., 1967). The modified Krebs perfusion buffer contained (in mM): 10 glucose, 1.75 CaCl₂, 118.5 NaCl, 4.7 KCl, 1.2 MgSO₄, 24.7 NaHCO₃, 0.5 EDTA, 12 mU/mL Insulin, and was gassed with 95% O₂-5% CO₂. Afterload was set by an 104 cm high aortic column (ID 3.18 mm), and hearts were allowed to beat at their own intrinsic heart rate (HR) in a sealed water jacketed chamber maintained at 37° C. Hearts were initially perfused for 15 minutes with normal perfusate, then were switched to a perfusate solution containing 300 nM Dobutamine for 10 minutes to challenge the hearts as previously described (Arany et al., 2005), and finally returned to normal perfusate for the final 15 minutes of perfusion. An open-type catheter (20-gauge needle) was inserted into the left ventricle for determination of heart rate (HR) ventricular pressures (LVDP) and their derivatives (+/−dP/dt) with all data collected and analyzed at a sampling rate of 200 Hz using PowerLab (ADInstruments, Colorado Springs, Colo.). The data acquisition system was calibrated daily against a known column of perfusate at 0 mmHg and 80 mmHg. An open-type catheter was chosen over an isovolumetric intraventricular balloon because of the small and varying size of the mouse heart and evidence that this system determines changes in end-diastolic/developed pressure as accurately as a balloon insertion (Pahor et al., 1985; Sutherland et al., 2003). Coronary flow (CF), normalized for heart wet weight, was determined by timed collection and cardiac external work (RPP) is defined as the product of HR and LVDP. At the end of the perfusion period the beating hearts were frozen in liquid nitrogen and stored at −80° C. for further analysis.

Noninvasive Measurements of Cardiac Function:

Magnetic resonance imaging (MRI) was performed after animals were weighed and anesthetized with intraperitoneal injections of Avertin (2.5% tribromoethanol and 0.8% 2-methyl-2-butanol in water, Sigma Chemicals) and monitored for normal respiratory function. The MRI scan was performed using a 1.5 T Philips Gyroscan NT whole body imaging system (Philips Medical Systems). The mouse was positioned supine in a 15 cm Petri dish and the electrocardiograph leads were attached to both front paws and one hindpaw. A standard finger coil was placed over the animal's chest and used for imaging the mouse heart. Heart rates were 380 to 450 beats per minute. Multislice, multiphase cine MRI was performed. Each study included a scout, coronal plane long axis of the left ventricle and a set of short axis acquisitions. Multiframe, short-axis gradient-echo sequences were used to measure LV end-systolic (LVESV) and diastolic volumes (LVEDV) as well as estimate LV mass and ejection fraction (EF). Four or five slices perpendicular to the long axis were obtained for each heart spanning from the apex to the base. The slice thickness was 1.6 mm with a 0.2 mm gap between slices. The pulse sequence was set for a heart rate of 210 bpm with nine cardiac phases and temporal resolution of 39 ms. The frame with the largest chamber dimensions was used as end diastole for mass and volume measurements and the image with the smallest chamber volume was used for end systolic measures. The LV mass, LVEDV, LVESV and EF were determined from images and calculated as previously described (Franco et al., 1998; Franco et al., 1999). Initial groups (n=10-15/group) of experimental animals were assessed serially at 3, 6, and 10 months.

RNA Extraction and RNA Dot Blot Analyses:

Anesthetized animals were perfused in situ with 10 ml of sterile PBS followed by 10 ml of RNAlater™ solution and hearts were immediately harvested. Atria were trimmed and the ventricles were immersed in RNAlater™ solution for 45 min at RT before frozen at −80° C. Total RNA was extracted and purified from 25-30 mg heart tissue using RNA Easy kit (QIAGEN, Valencia, Calif.), according to the manufacturer's instruction. RNA quality was monitored using Bio-analyzer and agarose gel electrophoresis. 1 μg of total RNA was diluted in Tris buffer, loaded and blotted on supercharged nylon membrane (BrighStar-plus, Ambion Inc.) using Biorad Biodot™ apparatus and the RNA was UV-cross linked to the membrane (Stratalinker, Stratagene). cDNA probes for atrial natriuretic factor (ANF), brain natriuretic factor (BNF), CryAB, and phospholamban (PLN) were generated using the following primer sets by PCR on mouse genomic DNA: ANF (325 bp); left, (SEQ ID NO:8); right, (SEQ ID NO:9); BNF (237 bp); left, (SEQ ID NO:10); right, (SEQ ID NO:11); CryAB (300 bp); left, (SEQ ID NO:12); right, (SEQ ID NO:13); and Phospholamban, (PLN, 583 bp); left, (SEQ ID NO:14), right, (SEQ ID NO:15).

PCR products from mouse genomic DNA were run on 1.5% agarose gel electrophoresis and fragments were purified using Qiaquick® gel extraction kit. RNA blots were probed with respective α[³²P] dATP radiolabeled DNA probes, hybridized in Ultrahyb (Ambion) solution for 16-18 hours and washed according to the manufacturer's instruction. Membranes were then exposed to radiosensitive X-ray film (Hyblot CL Autoradiography, Denville Scientific Inc.) to detect hybridization signals using autoradiography for 16-24 hours. Levels of mRNA expression were obtained from scanned images and quantified using Image J analysis software.

Antioxidant Enzyme Activity Assays:

Cytosolic activities of selected antioxidant enzymes were measured using commercially available kits from Bioxitech (OxisResearch). Catalase activity was determined using the Catalase-520™ assay in a two-step procedure (Aebi, 1984). Dismutation of hydrogen peroxide (H₂O₂) to water and molecular oxygen is proportional to the concentration of catalase. Diluted homogenates containing catalase were incubated in the presence of known concentration of H₂O₂. After incubation for 60 seconds, reaction was quenched with sodium azide. The amount of H₂O₂ remaining in the reaction mixture was then determined by the oxidative coupling reaction of 4-aminophenazone (4-aminoantipyrene, AAP) and 3,5-dichloro-2-hydroxybenzenesulfonic acid (DHBS) in the presence of H₂O₂ and catalyzed by horseradish peroxidase (HRP) and the resulting quinoneimine dye was measured at 520 nm.

The GPx-340™ assay is an indirect measure of the activity of cytosolicGPx (Ursini et al., 1995). Oxidized glutathione (GSSG), produced in reduction of organic peroxide by c-GPx, is recycled to its reduced state by the enzyme glutathione reductase (GSH-R). The oxidation of NADPH to NADP+ is accompanied by a decrease in absorbance at 340 nm (A340) providing a spectrophotometric means for monitoring GPx enzyme activity. To assay c-GPx, heart homogenate was added to a solution containing glutathione, glutathione reductase, and NADPH. The enzymatic reaction was initiated by adding the substrate, tert-butyl hydroperoxide, and the A340 was recorded. The rate of decrease in the A340 is directly proportional to the GPx activity in the sample. The GR-340 assay is based on the oxidation of NADPH to NADP+ catalyzed by a limiting concentration of glutathione reductase (Beutler, 1969). One unit GSH-R activity in the homogenates is defined as the amount of enzyme catalyzing the reduction of one micromole of GSSG per minute at pH 7.6 and 25° C. The reduction of GSSG, determined indirectly by the measurement of the consumption of NADPH, decreases the absorbance at 340 nm (A340) as a function of time.

Determination of Lipid Peroxides:

Lipid peroxidation is a well-established mechanism of cellular injury and is used as an indicator of oxidative stress in cells and tissues in vivo. Lipid peroxides and modified products derived from polyunsaturated fatty acids are unstable and decompose to form complex compounds such as reactive carbonyl, the most abundant of which is malondialdehyde (MDA). The lipid peroxidation products, as MDA, were measured in the heart homogenates using the thiobarbituric acid (TBA) reaction (Esterbauer and Cheeseman, 1990). In brief, 2.0 ml of 20% TCA supernatants from heart homogenates were mixed with 1.0% TBA reagent and boiled in a water bath for 15 minutes. The absorbance of the chromogen produced was measured at 532 nm in a Beckman UV-visible spectrophotometer.

Immunochemical Quantitation of Protein Carbonyls:

Heart homogenates were prepared in 20 mM Tris-HCl buffer, pH 6.8 containing 0.2% SDS and treated with 10 mM Di-Nitro Phenyl Hydrazine (DNPH) as described previously (Yan and Sohal, 2000). The homogenates with DNPH were separated in 10% SDS-PAGE and probed against anti-DNPH antibody (Keller et al., 1993; Shacter et al., 1994). Nitrocellulose blots were incubated in 50 ml of 5% non-fat dried milk overnight at 4° C. and then washed with Trisbuffered saline (20 mM Tris, 500 mm NaCl pH 7.5), containing 0.1% Tween-20 (TBST), rinsed for 3 times (10 min each) and were incubated with primary rabbit anti-DNP antibody (1:2000 in TBST containing 0.2% BSA) for 2 hours at room temperature. Washes were repeated in TBST for 3 times before incubation with secondary rabbit IgG (diluted 1:25000 in TBST containing 0.2% BSA) for 1 hour at room temperature. After 5 washes (10 min each) in TBST, the blots were then treated with enhanced chemiluminescence (ECL, Amersham) detection kit. The signals for oxidized proteins were quantified using Image J densitometry software.

### 4. Example 4

### Reductive Stress in Human Multisystem Protein Aggregation Diseases

Crystallins (α, ζ, γ, etc.) are abundant soluble proteins in the ocular lens where they provide essential functions in the organ's requirements for chaperone-dependent protein quality control, structural integrity and light transparency. Several disease-causing mutations of CryAB have been identified as shown in Table 16 (Pilotto, 2006; Liu, 2006 #5561) but the R120G mutation of hCryAB causes an autosomal dominant, multisystem disorder that includes cataracts and cardiomyopathy (Vicart, 1998; Fardeau, 1978). Cataracts per se are widely believed to be aggregates of partially misfolded crystallins, resulting from protein denaturation and damage from oxidative stress. Because the defective chaperone R120GCryAB is prone to misfolding and self-aggregation, DRM can comprise a loss-of-function mutation characterized by protein aggregates containing desmin and other misfolded proteins (Bova, 1999 #4575). Alternatively, toxic gain-of-function mutations can lead to excess reducing equivalents. Thus, the principles and mechanistic insights gained from studies of hR120CryAB cardiomyopathy are applied to other CryAB mutants using approaches disclosed herein.

TABLE 16
CryAB Mutations Associated with Multisystem Human Disease

| Description | P20S | R56A | R120G | D140N | CPP2 | Q151X | G154S | R157H |
|---|---|---|---|---|---|---|---|---|
| Cardiomyopathy | — | ND | X | — | — | — | X | X |
| | | | | | | | DCM | DCM |
| Cataract | X | ND | X | X | X | — | — | — |
| Skeletal | — | ND | X | — | — | X | — | — |
| myopathy | | | | | | | | |
| Onset | Late | ND | Late | — | Late | Late | Late | Late |
| Respiratory | X | ND | X | — | — | X | — | — |
| weakness | | | | | | | | |
| Severity index | Severe | ND | Severe | — | — | — | — | — |
| EM dense | — | ND | X | — | — | — | — | — |
| aggregation | | | | | | | | |

i. Methodology and Analysis:

It is determined if disease-causing CryAB mutants induce G6PD upregulation in cultured cells. By design, CryAB mutants not linked presently to cardiomyopathy are selected. First, it is determined if CryAB mutants associated with cataracts and other multisystem defects such as respiratory and skeletal muscle weakness can also trigger reductive stress in vitro (Table 16). Second, this strategy is an efficient approach towards taking fullest advantage of the Drosophila animal model disclosed herein.

Molecular cloning of the disease-causing CryAB mutations are accomplished using standard techniques such as those disclosed herein. cDNAs under the control of β-actin, a strong constitutive promoter, are expressed with and without affinity tags (Myc and FLAG) to more easily detect and facilitate cellular and molecular studies in Hela the myogenic C2C12 cell line. Glutathione measurements, antioxidant enzyme activity assays, and glucose-6-phosphate dehydrogenase activity are determined as disclosed herein. Total glutathione and oxidized glutathione (samples derivatized with 2-vinyl pyridine) are measured by a standard recycling assay based on the reduction of 5,5-dithiobis-2-nitrobenzoic acid in the presence of glutathione reductase and NADPH (Griffith, 1980). As potential toxic gain-of-function mutations, it is possible that several CryAB mutants exert disease pathogenesis via unrecognized molecular targets, besides G6PD. Alternatively, such studies can be useful diagnostic tools, provide a mechanistic basis to screen for subclinical disease, and, in principle, enable us to predict organ-specific disease progression or prevention.

ii. Characterize Disease-Causing CryAB Mutants (Aim 1) for Protein Aggregation and Cell Toxicity.

To verify protein aggresomes, the following antibodies and reagents are used: an anti-CryAB polyclonal antibody, which recognizes both the mouse and human proteins, was raised against residues 164-175 of human CryAB. Rabbit anti-Hsp25, anti-Hsp70, anti-Hsp90 (StressGen, Victoria, BC, Canada) and rabbit anti-G6PD (Novus Bio.), anti-catalase, anti-glutathione peroxidase, anti-glutathione reductase (AbCam), and gamma-GCS/glutamate cysteine ligase-Abl (Labvision, Neomarkers, CA) are purchased from commercial vendors. The approaches for assessment and characterization of cellular toxicity is disclosed herein.

It is unlikely that all other CryAB mutants mediate toxicity via identical pathways as R120GCryAB so the focus on identification of new targets or common disease-causing domains can yield new lines of investigations to be characterized at the genetic, structural, biochemical, and clinical levels in future PPGs or multidisciplinary team-based grants.

### 5. Example 5

### Modeling Human R120GCryAB Protein Aggregation in Drosophila

Disclosed is a model of protein aggregation in Drosophila by reproducing essential aspects of the phenotype in the fly. Also disclosed is a confirmation of the similarity to the human disease by examining the effects of environmental and candidate genetic modifiers. This validated phenotype model is used to screen other CryAB mutants in order to gain a full understanding of the biological mechanisms that underlie the disease phenotypes. This an also allow testing of potential therapeutic compounds.

To reproduce the disease phenotype in Drosophila a two-pronged approach is used. First, the disease allele is expressed with the strongest and most widespread phenotype (R120G) in Drosophila under control of the Gal-4-UAS system. This system provides for the conditional and regulated expression of transgenes in virtually any tissue of the fly. Expression can focus on in the compound eye, because the eye has a highly stereotypical pattern that is very sensitive for detecting disruptions of cellular function during development, and because the eye is dispensable for life. This system has proven utility for detecting interactions via genetic screens. The mutant protein is also expressed in flight muscles and in heart muscles to more precisely mimic the myopathies. The affected cells are examined for the presence of dense protein aggregates to validate this aspect of the model. The protein is expressed in whole flies, and if viable, they are tested for altered glutathione levels.

In case no phenotype is readily observed, and expression of the R120G protein is verified, several approaches are used to generate a visible and genetically useful phenotype. In some circumstances Hsp70 cooperates with Hsp27 (Lee and Vierling 2000). Ectopically-expressed R120G is combined with Hsp70 gene deletions or duplications to test whether decreasing or increasing the dose of a partner can enhance the mutant phenotype. It is also possible that the R120G mutant can cause protein aggregation in Drosophila, but have little obvious phenotype because its effect will occur only after a period of aging. After expression in the eye, eye cells of aged adults are examined for the presence of dense protein aggregates. If present, R120G expression is combined with G6PD overexpression to accelerate their appearance and potential phenotype.

The second approach to reproduce DRM in Drosophila is to use gene targeting methods (Rong et al. 2002) to precisely engineer the R120G mutation into the fly homologs of the αB-crystallin gene. There are two genes that are closely and almost equally related to CryAB: Hsp27 and l(2)efl. Hsp27 can be the most appropriate choice, given that previous work has shown that overexpression of Drosophila Hsp27 can cause increased glutathione levels and that mutation of the conserved arginine residue in the α-crystallin domains of four small MW Hsps (α-crystallin of αA-, αB-crystallin, HspB8 and hamster Hsp27) cause protein aggregates (Chavez Zobel, 2005). So, it can be mutated first. It can also be necessary to engineer mutations in the remaining homologs also, and this can be accomplished with current technology. The mutant flies for dominant and recessive effects are examined, particularly with respect to viability, lifespan, and fertility. If single mutants have no phenotype, double mutants are examined as well. Cells are examined for the presence of dense protein aggregates.

To validate the disease model the response to genetic and environmental modification es examined. First, the effects of mutations in G6PD are tested for suppression of phenotypes. Second, the effect of an oxygen rich environment, or oxidative stressors such as paraquat or peroxide is examined. If it can be demonstrated that these factors also suppress the phenotypes, similar to what has been seen in the mouse with G6PD mutants, the model is considered valid.

In cultured mouse cells, glutathione levels increase upon transgenic expression of crystallin-domain proteins such as Drosophila Hsp27 (Mehlen et al. 1996). This indicates the possibility that the R120G mutant is hyperactive. Excess Hsp27 activity can generate an imbalance between Hsp27 and partner chaperones. In Drosophila it is a relatively simple matter to change the effective functional dose of a gene. It is determined whether over-expression of wild-type Hsp27 or l(2)efl produces a phenotype similar to the R120G mutation. Such studies are highly informative as to whether Hsp27 overexpression is necessary and sufficient for the R120G phenotype. If not, focus is on models that invoke aberrant activity of the R120G mutant.

### G. REFERENCES

Adachi, T., Pimentel, D. R., Heibeck, T., Hou, X., Lee, Y. J., Jiang, B., Ido, Y., and Cohen, R. A. 2004. S-glutathiolation of Ras mediates redox-sensitive signaling by angiotensin II in vascular smooth muscle cells. J Biol Chem 279:29857-29862.
Aebi, H.1984. Catalase iin vitro. Methods in Enzymology 105:121-126.
Amaratunga D, and Cabrera J. Exploration and analysis of DNA microarray and protein array data. Hoboken, N.J.: John Wiley, 2004, p. xiv, 246 p.
Arany, Z., He, H., Lin, J., Hoyer, K., Handschin, C., Toka, O., Ahmad, F., Matsui, T., Chin, S., Wu, P. H., et al. 2005. Transcriptional coactivator PGC-1 alpha controls the energy state and contractile function of cardiac muscle. Cell Metab 1:259-271.
Arrigo, A. P., Paul, C., Ducasse, C., Sauvageot, O., and Kretz-Remy, C. 2002. Small stress proteins: modulation of intracellular redox state and protection against oxidative stress. Prog Mol Subcell Biol 28:171-184.
Arrigo, A. P., Virot, S., Chaufour, S., Firdaus, W., Kretz-Remy, C., and Diaz-Latoud, C. 2005. Hsp27 consolidates intracellular redox homeostasis by upholding glutathione in its reduced form and by decreasing iron intracellular levels. Antioxid Redox Signal 7:414-422.
Baek, S. H., Min, J. N., Park, E. M., Han, M. Y., Lee, Y. S., Lee, Y. J., and Park, Y. M. 2000. Role of small heat shock protein HSP25 in radioresistance and glutathione-redox cycle. J Cell Physiol 183:100-107.
Belke, D. D., Betuing, S., Tuttle, M. J., Graveleau, C., Young, M. E., Pham, M., Zhang, D., Cooksey, R. C., McClain, D. A., Litwin, S. E., Taegtmeyer, H., Severson, D., Kahn, C. R. & Abel, E. D. (2002) J Clin Invest 109, 629-39.
Bell, J. (2004). Predicting disease using genomics. Nature 429, 453-456.
Bengtsson H, Jonsson G, and Vallon-Christersson J. Calibration and assessment of channel-specific biases in microarray data with extended dynamical range. BMC Bioinformatics 5: 177, 2004.
Benjamin I J, and Schneider M D. Learning from failure: congestive heart failure in the postgenomic age. The Journal of clinical investigation 115: 495-499, 2005. Benjamin, I. J., and McMillan, D. R. 1998. Stress (Heat Shock) Proteins: Molecular Chaperones in Cardiovascular Biology and Disease. Circ Res 83:117-132.
Benjamin, I. J., and Schneider, M. D. (2005). Learning from failure: congestive heart failure in the postgenomic age. J. Clin. Invest. 115, 495-499.
Benjamini Y, and Hochberg Y. Controlling the false discovery rate: a practical and powerful approach to multiple testing. Journal of the Royal Statistical Society, Series B 57: 289-300, 1995.
Bennardini, F., Wrzosek, A. & Chiesi, M. (1992) Circ Res 71, 288-94.
Beutler, E. 1969. Effect of flavin compounds on glutathione reductase activity: in vivo and iin vitro studies. J Clin Invest 48:1957-1966.
Bjorkoy G, Lamark T, and Johansen T. p62/SQSTM1: a missing link between protein aggregates and the autophagy machinery. Autophagy 2: 138-139, 2006.
Blaxall B C, Spang R, Rockman H A, and Koch W J. Differential myocardial gene expression in the development and rescue of murine heart failure. Physiological genomics 15: 105-114, 2003. Boston, D. R., Koyama, T., Rodriguez-Larrain, J., Zou, A., Su, Z., and Barry, W. H.1998. Effects of angiotensin II on intracellular calcium and contracture in metabolically inhibited cardiomyocytes. J Pharmacol Exp Ther 285:716-723.
Blaxall, B. C., Spang, R., Rockman, H. A., and Koch, W. J. (2003). Differential myocardial gene expression in the development and rescue of murine heart failure. Physiol Genomics 15, 105-114.
Bleumink, G. S., Knetsch, A. M., Sturkenboom, M. C., Straus, S. M., Hofman, A., Deckers, J. W., Witteman, J. C., and Stricker, B. H. (2004). Quantifying the heart failure epidemic: prevalence, incidence rate, lifetime risk and prognosis of heart failure The Rotterdam Study. Eur Heart J 25, 1614-1619.
Boston, D. R., Koyama, T., Rodriguez-Larrain, J., Zou, A., Su, Z., and Barry, W. H. (1998). Effects of angiotensin II on intracellular calcium and contracture in metabolically inhibited cardiomyocytes. J Pharmacol Exp Ther 285, 716-723.
Bosze Z, Hiripi L, Carnwath J W, et al. The transgenic rabbit as model for human diseases and as a source of biologically active recombinant proteins. Transgenic Res. October 2003; 12(5):541-553.
Bova, M. P., Yaron, O., Huang, Q., Ding, L., Haley, D. A., Stewart, P. L., and Horwitz, J. 1999. Mutation R120G in alphaB-crystallin, which is linked to a desmin-related myopathy, results in an irregular structure and defective chaperone-like function. Proc Natl Acad Sci USA 96:6137-6142.
Braunwald, E., and Bristow, M. R. (2000). Congestive heart failure: fifty years of progress. Circulation 102, IV1 4-23.
Buckingham, M., Meilhac, S., and Zaffran, S. (2005). Building the mammalian heart from two sources of myocardial cells. Nat Rev Genet. 6, 826-835.
Bukau, B., Weissman, J., and Horwich, A. (2006). Molecular chaperones and protein quality control. Cell 125, 443-451.
Carper, D., John, M., Chen, Z., Subramanian, S., Wang, R., Ma, W., and Spector, A. 2001. Gene expression analysis of an H(2)O(2)-resistant lens epithelial cell line. Free Radic Biol Med 31:90-97.
Chance, B., Sies, H., and Boveris, A. (1979). Hydroperoxide metabolism in mammalian organs. Physiol Rev 59, 527-605.
Chance, B., Sies, H., and Boveris, A. 1979. Hydroperoxide metabolism in mammalian organs. Physiol Rev 59:527-605.
Chen, Q., Liu, J. B., Horak, K. M., Zheng, H., Kumarapeli, A. R., Li, J., Li, F., Gerdes, A. M., Wawrousek, E. F. & Wang, X. (2005) Circ Res 97, 1018-26.
Christians, E. S., Yan, L. J., and Benjamin, I. J. 2002. Heat shock factor 1 and heat shock proteins: Critical partners in protection against acute cell injury. Crit Care Med 30:S43-S50.
Clemen, C. S., Fischer, D., Roth, U., Simon, S., Vicart, P., Kato, K., Kaminska, A. M., Vorgerd, M., Goldfarb, L. G., Eymard, B., et al. (2005). Hsp27-2D-gel electrophoresis is a diagnostic tool to differentiate primary desminopathies from myofibrillar myopathies. FEBS Lett. 579, 3777-3782.
Cocco, P., Todde, P., Formera, S., Manca, M. B., Manca, P., and Sias, A. R. 1998. Mortality in a cohort of men expressing the glucose-6-phosphate dehydrogenase deficiency. Blood 91:706-709.
Dalakas, M. C., Park, K. Y., Semino-Mora, C., Lee, H. S., Sivakumar, K., and Goldfarb, L. G. 2000. Desmin myopathy, a skeletal myopathy with cardiomyopathy caused by mutations in the desmin gene. N Engl J Med 342:770-780.
Danial, N. N., and Korsmeyer, S. J. (2004). Cell death: critical control points. Cell 116, 205-219.
Dawson, T. L., Gores, G. J., Nieminen, A. L., Herman, B., and Lemasters, J. J. 1993. Mitochondria as a source of reactive oxygen species during reductive stress in rat hepatocytes. Am J Physiol 264:C961-967.
Deng, M. C., Eisen, H. J., Mehra, M. R., Billingham, M., Marboe, C. C., Berry, G., Kobashigawa, J., Johnson, F. L., Starling, R. C., Murali, S., et al. (2006). Noninvasive discrimination of rejection in cardiac allograft recipients using gene expression profiling. Am J Transplant 6, 150-160.
DiBianco, R. (2003). Update on therapy for heart failure. Am J Med 115, 480-488.
Donahue, M. P., Marchuk, D. A., and Rockman, H. A. (2006). Redefining heart failure: the utility of genomics. Journal of the American College of Cardiology 48, 1289-1298.
Dunselman, P. H., Kuntze, C. E., van Bruggen, A., Beekhuis, H., Piers, B., Scaf, A. H., Wesseling, H., and Lie, K. I. (1988). Value of New York Heart Association classification, radionuclide ventriculography, and cardiopulmonary exercise tests for selection of patients for congestive heart failure studies. Am Heart J 116, 1475-1482.
Esterbauer, H., and Cheeseman, K. H. (1990). Determination of aldehydic lipid peroxidation products: Malondialdehyde and 4-hydroxynonanol. Methods in Enzymology 186, 407-420.
Esterbauer, H., Schaur, R. J., and Zollner, H.1991. Chemistry and biochemistry of 4-hydroxynonenal, malonaldehyde and related aldehydes. Free Radic Biol Med 11:81-128.
Evans, W. E., and McLeod, H. L. (2003). Pharmacogenomics—drug disposition, drug targets, and side effects. N Engl J Med 348, 538-549.
Fardeau, M., Godet-Guillain, J., Tome, F. M., Collin, H., Gaudeau, S., Boffety, C., and Vernant, P. 1978. [A new familial muscular disorder demonstrated by the intra-sarcoplasmic accumulation of a granulo-filamentous material which is dense on electron microscopy (author's transl)]. Rev Neurol (Paris) 134:411-425.
Fardeau, M., Godet-Guillain, J., Tome, F. M., Collin, H., Gaudeau, S., Boffety, C., and Vernant, P. (1978). Rev. Neurol. (Paris) 134, 411-425. Finner, H. (1993). On a monotonicity problem with in step-down multiple test procedures. J. Am. Stat. Assoc. 88, 920-923.
Franco, F., Dubois, S. K., Peshock, R. M., and Shohet, R. V. 1998. Magnetic resonance imaging accurately estimates LV mass in a transgenic mouse model of cardiac hypertrophy. Am J Physiol 274:H679-683.
Franco, F., Thomas, G. D., Giroir, B., Bryant, D., Bullock, M. C., Chwialkowski, M. C., Victor, R. G., and Peshock, R. M. 1999. Magnetic resonance imaging and invasive evaluation of development of heart failure in transgenic mice with myocardial expression of tumor necrosis factor-alpha. Circulation 99:448-454.
Fratelli M, Goodwin L O, Orom U A, Lombardi S, Tonelli R, Mengozzi M, and Ghezzi P. Gene expression profiling reveals a signaling role of glutathione in redox regulation. Proceedings of the National Academy of Sciences of the United States of America 102: 13998-14003, 2005.
Gething M J, and Sambrook J. Protein folding in the cell. Nature 355: 33-45, 1992.
Ghyczy M, Boros M. Electrophilic methyl groups present in the diet ameliorate pathological states induced by reductive and oxidative stress: a hypothesis. Br J Nutr. April 2001; 85(4):409-414.
Ghyczy M, Torday C, and Boros M. Simultaneous generation of methane, carbon dioxide, and carbon monoxide from choline and ascorbic acid: a defensive mechanism against reductive stress? Faseb J 17: 1124-1126, 2003.
Giordano F J. Oxygen, oxidative stress, hypoxia, and heart failure. The Journal of clinical investigation 115: 500-508, 2005.
Goebel, H. H., and Warlo, I. 2000. Gene-related protein surplus myopathies. Mol Genet Metab 71:267-275.
Goldfarb, L. G., Park, K. Y., Cervenakova, L., Gorokhova, S., Lee, H. S., Vasconcelos, O., Nagle, J. W., Semino-Mora, C., Sivakumar, K., and Dalakas, M. C. 1998. Missense mutations in desmin associated with familial cardiac and skeletal myopathy. Nat Genet 19:402-403.
Goldfarb, L. G., Vicart, P., Goebel, H. H., and Dalakas, M. C. (2004). Desmin myopathy. Brain 127, 723-734.
Gores, G. J., Flarsheim, C. E., Dawson, T. L., Nieminen, A. L., Herman, B., and Lemasters, J. J. (1989). Swelling, reductive stress, and cell death during chemical hypoxia in hepatocytes. Am J Physiol 257, C347-354.
Griendling, K. K., and FitzGerald, G. A. 2003. Oxidative stress and cardiovascular injury: Part I: basic mechanisms and in vivo monitoring of ROS. Circulation 108:1912-1916.
Griffith, O. W. 1980. Determination of glutathione and glutathione disulfide using glutathione reductase and 2-vinylpyridine. Anal Biochem 106:207-212.
Griffiths, G. 1993. Fine Structure Immunohistochemistry. New York: Springer-Verlag.
Grupp, I. L., Subramaniam, A., Hewett, T. E., Robbins, J., and Grupp, G. (1993). Comparison of normal, hypodynamic, and hyperdynamic mouse hearts using isolated work-performing heart preparations. Am J Physiol 265, H1401-1410.
Gupte, S. A., Levine, R. J., Gupte, R. S., Young, M. E., Lionetti, V., Labinskyy, V., Floyd, B. C., Ojaimi, C., Bellomo, M., Wolin, M. S., et al. (2006). Glucose-6-phosphate dehydrogenase-derived NADPH fuels superoxide production in the failing heart. J. Mol. Cell. Cardiol. 41, 340-349.
Handy, D. E., Hang, G., Scolaro, J., Metes, N., Razaq, N., Yang, Y., and Loscalzo, J. (2006). Aminoglycosides decrease glutathione peroxidase-1 activity by interfering with selenocysteine incorporation. J. Biol. Chem. 281, 3382-3388.
Hansen, J. M., Go, Y. M., and Jones, D. P. (2006). Nuclear and mitochondrial compartmentation of oxidative stress and redox signaling. Annu. Rev. Pharmacol. Toxicol. 46, 21 5-234.
Hochman, J. H., Schindler, M., Lee, J. G., and Ferguson-Miller, S. (1982). Lateral mobility of cytochrome c on intact mitochondrial membranes as determined by fluorescence redistribution after photo-bleaching. Proc. Natl. Acad. Sci. USA 79, 6866-6870.
Hofmann, M., Wollert, K. C., Meyer, G. P., Menke, A., Arseniev, L., Hertenstein, B., Ganser, A., Knapp, W. H., and Drexler, H. (2005). Monitoring of bone marrow cell homing into the infarcted human myocardium. Circulation 111, 2198-2202.
Hollander, J. M., Lin, K. M., Scott, B. T., and Dillmann, W. H. 2003. Overexpression of PHGPx and HSP60/10 protects against ischemia/reoxygenation injury. Free Radic Biol Med 35:742-751.
Ido, Y., Kilo, C., and Williamson, J. R. 1997. Cytosolic NADH/NAD+, free radicals, and vascular dysfunction in early diabetes mellitus. Diabetologia 40 Suppl 2:S115-117.
Inagaki N, Hayashi T, Arimura T, et al. Alpha B-crystallin mutation in dilated cardiomyopathy. Biochem Biophys Res Commun Apr. 7 2006; 342(2):379-386.
Iwaki A, Iwaki T, Goldman J E, Ogomori K, Tateishi J, and Sakaki Y. Accumulation of alpha B-crystallin in brains of patients with Alexander's disease is not due to an abnormality of the 5′-flanking and coding sequence of the genomic DNA. Neurosci Lett 140: 89-92, 1992.
Jacobsen D W, Gatautis V J, Green R, et al. Rapid HPLC determination of total homocysteine and other thiols in serum and plasma: sex differences and correlation with cobalamin and folate concentrations in healthy subjects. Clin Chem. June 1994; 40(6):873-881.
Jessup, M., and Brozena, S. (2003). Heart failure. N Engl J Med 348, 2007-2018.
Johnston, J. A., Ward, C. L., and Kopito, R. R. (1998). Aggresomes: A cellular response to misfolded proteins. J. Cell Biol. 143, 1883-1 898. Kappe, G., Franck, E., Verschuure, P., Boelens, W. C., Leunissen, J. A., and de Jong, W. W. (2003). The human genome encodes 10 alphacrystallin-related small heat shock proteins: HspB1-10. Cell Stress Chaperones 8, 53-61.
Kadono, T., Zhang, X. Q., Srinivasan, S., Ishida, H., Barry, W. H., and Benjamin, I. J. (2006). CRYAB and HSPB2 deficiency increases myocyte mitochondrial permeability transition and mitochondrial calcium uptake. J Mol Cell Cardiol 40, 783-789.
Kamradt, M. C., Chen, F. & Cryns, V. L. (2001) J Biol Chem 276, 16059-63.
Kanehisa M, Goto S, Hattori M, Aoki-Kinoshita K F, Itoh M, Kawashima S, Katayama T, Araki M, and Hirakawa M. From genomics to chemical genomics: new developments in KEGG. Nucleic acids research 34: D354-357, 2006.
Kato, K., Shinohara, H., Kurobe, N., Inaguma, Y., Shimizu, K. & Ohshima, K. (1991) Biochim Biophys Acta 1074, 201-8.
Keller, R. J., Halmes, N. C., Hinson, J. A., and Pumford, N. R. 1993 Immunochemical detection of oxidized proteins. Chem Res Toxicol 6:430-433.
Klemenz, R., Frohli, E., Steiger, R. H., Schafer, R., and Aoyama, A. (1991). Alpha B-crystallin is a small heat shock protein. Proc. Natl. Acad. Sci. USA 88, 3652-3656.
Kletzien, R. F., Harris, P. K., and Foellmi, L. A. (1994). Glucose-6-phosphate dehydrogenase: a “housekeeping” enzyme subject to tissue-specific regulation by hormones, nutrients, and oxidant stress. FASEB J. 8, 174-181.
Knowlton, A. A., Kapadia, S., Torre-Amione, G., Durand, J. B., Bies, R., Young, J., and Mann, D. L. (1998). Differential expression of heat shock proteins in normal and failing human hearts. J. Mol. Cell. Cardiol. 30, 811-818.
Kumar M S, Reddy P Y, Sreedhar B, Reddy G B. 2005. Alphab-crystallin-assisted reactivation of glucose-6-phosphate dehydrogenase upon refolding. Biochem J. 15; 391(Pt 2):335-41.
Kumar, L. V., Ramakrishna, T., and Rao, C. M. 1999. Structural and functional consequences of the mutation of a conserved arginine residue in alphaA and alphaB crystallins. J Biol Chem 274:24137-24141.
Lash L H. Mitochondrial glutathione transport: physiological, pathological and toxicological implications. Chemico-biological interactions 163: 54-67, 2006
Lee, C. Y. 1982. Glucose-6-phosphate dehydrogenase from mouse. Methods Enzymol 89 Pt D:252-257.
Lee, R. T., Bloch, K. D., Pfeffer, J. M., Pfeffer, M. A., Neer, E. J. & Seidman, C. E. (1988) J Clin Invest 81, 431-4.
Lee, G. J., and E. Vierling, 2000 A small heat shock protein cooperates with heat shock protein 70 systems to reactivate a heat-denatured protein. Plant Physiol. 122, 189-198.
Leopold J A, Zhang Y Y, Scribner A W, Stanton R C, Loscalzo J. 2003. Glucose-6-phosphate dehydrogenase overexpression decreases endothelial cell oxidant stress and increases bioavailable nitric oxide. Arterioscler Thromb Vasc Biol. 1; 23(3):411-7.
Leopold, J. A., Cap, A., Scribner, A. W., Stanton, R. C., and Loscalzo, J. (2001). Glucose-6-phosphate dehydrogenase deficiency promotes endothelial oxidant stress and decreases endothelial nitric oxide bioavailability. FASEB J. 15, 1771-1773.
Leopold, J. A., Dam, A., Maron, B. A., Scribner, A. W., Liao, R., Handy, D. E., Stanton, R. C., Pitt, B., and Loscalzo, J. (2007). Aldosterone impairs vascular reactivity by decreasing glucose-6-phosphate dehydrogenase activity. Nat. Med. 13, 189-197.
Leopold, J. A., Walker, J., Scribner, A. W., Voetsch, B., Zhang, Y. Y., Loscalzo, A. J., Stanton, R. C., and Loscalzo, J. (2003). Glucose-6-phosphate dehydrogenase modulates vascular endothelial growth factor-mediated angiogenesis. J. Biol. Chem. 278, 32100-32106.
Liggett, S. B. (2001). Pharmacogenetic applications of the Human Genome project. Nat Med 7, 281-283.
Lipinski B. Evidence in support of a concept of reductive stress. Br J Nutr. January 2002; 87(1):93-94
Liu, M., Ke, T., Wang, Z., Yang, Q., Chang, W., Jiang, F., Tang, Z., Li, H., Ren, X., Wang, X., et al. (2006). Identification of a CRYAB mutation associated with autosomal dominant posterior polar cataract in a Chinese family. Invest. Ophthalmol. Vis. Sci. 47, 3461-3466.
Liu, P. P., and Mason, J. W. (2001). Advances in the understanding of myocarditis. Circulation 104, 1076-1082.
Lockwood, T. D. (2005) Antioxid Redox Signal 7, 982-98.
Lowes, B. D., Zolty, R., Minobe, W. A., Robertson, A. D., Leach, S., Hunter, L. & Bristow, M. R. (2006) J Heart Lung Transplant 25, 579-88.
Maisel, A. S., and McCullough, P. A. (2003). Cardiac natriuretic peptides: a proteomic window to cardiac function and clinical management. Rev Cardiovasc Med 4 Suppl 4, S3-12.
Maloyan, A., Sanbe, A., Osinska, H., Westfall, M., Robinson, D., Imahashi, K., Murphy, E., and Robbins, J. (2005). Mitochondrial dysfunction and apoptosis underlie the pathogenic process in alphaB-crystallin desmin-related cardiomyopathy. Circulation 112, 3451-3461.
Marian A J, Wu Y, Lim D S, et al. A transgenic rabbit model for human hypertrophic cardiomyopathy. J Clin Invest. December 1999; 104(12):1683-1692.
Matsui, R., Xu, S., Maitland, K. A., Hayes, A., Leopold, J. A., Handy, D. E., Loscalzo, J., and Cohen, R. A. 2005. Glucose-6 phosphate dehydrogenase deficiency decreases the vascular response to angiotensin II. Circulation 112:257-263.
McNamara, D. M., Holubkov, R., Janosko, K., Palmer, A., Wang, J. J., MacGowan, G. A., Murali, S., Rosenblum, W. D., London, B., and Feldman, A. M. (2001). Pharmacogenetic interactions between beta-blocker therapy and the angiotensin-converting enzyme deletion polymorphism in patients with congestive heart failure. Circulation 103, 1644-1648.
McNamara, D. M., Holubkov, R., Postava, L., Janosko, K., MacGowan, G. A., Mathier, M., Murali, S., Feldman, A. M., and London, B. (2004). Pharmacogenetic interactions between angiotensin-converting enzyme inhibitor therapy and the angiotensin-converting enzyme deletion polymorphism in patients with congestive heart failure. Journal of the American College of Cardiology 44, 2019-2026.
Mehlen P, Kretz-Remy C, Preville X, et al. Human hsp27, Drosophila hsp27 and human alphaB-crystallin expression-mediated increase in glutathione is essential for the protective activity of these proteins against TNFalpha-induced cell death. Embo J. 1996; 15(11):2695-2706.
Mehlen, P., Schulze-Osthoff, K., and Arrigo, A. P. (1996). Small stress proteins as novel regulators of apoptosis. Heat shock protein 27 blocks Fas/APO-1- and staurosporine-induced cell death. J. Biol. Chem. 271, 16510-16514.
Mehra, M. R. (2005). The emergence of genomic and proteomic biomarkers in heart transplantation. J Heart Lung Transplant 24, S213-218.
Meister A, and Anderson M E. Glutathione. Annual review of biochemistry 52: 711-760, 1983.
Morimoto, R. I. (1998). Regulation of the heat shock transcriptional response: cross talk between a family of heat shock factors, molecular chaperones, and negative regulators. Genes Dev. 12, 3788-3796.
Morita, H., Seidman, J., and Seidman, C. E. (2005). Genetic causes of human heart failure. The Journal of Clinical Investigation 115, 518-526.
Morita, H., Seidman, J., and Seidman, C. E. 2005. Genetic causes of human heart failure. J Clin Invest 115:518-526.
Neely, J. R., Liebermeister, H., Battersby, E. J., and Morgan, H. E. 1967. Effect of pressure development on oxygen consumption by isolated rat heart. Am J Physiol 212:804-814.
Pahor, M., Di Gennaro, M., Cocchi, A., Bernabei, R., Carosella, L., and Carbonin, P. 1985. Age-related incidence of reperfusion- and reoxygenation-induced ventricular tachyarrhythmias in the isolated rat heart. Gerontology 31:15-26.
Perng, M. D., Muchowski, P. J., van Den, I. P., Wu, G. J., Hutcheson, A. M., Clark, J. I., and Quinlan, R. A. 1999. The cardiomyopathy and lens cataract mutation in alphaB-crystallin alters its protein structure, chaperone activity, and interaction with intermediate filaments iin vitro. J Biol Chem 274:33235-33243.
Perrone G G, Grant C M, and Dawes I W. Genetic and environmental factors influencing glutathione homeostasis in Saccharomyces cerevisiae. Molecular biology of the cell 16: 218-230, 2005.
Pilotto A, Marziliano N, Pasotti M, et al. alphaB-crystallin mutation in dilated cardiomyopathies: low prevalence in a consecutive series of 200 unrelated probands. Biochem Biophys Res Commun. Aug. 11 2006; 346(4):1115-1117.
Preville, X., Salvemini, F., Giraud, S., Chaufour, S., Paul, C., Stepien, G., Ursini, M. V., and Arrigo, A. P. 1999. Mammalian small stress proteins protect against oxidative stress through their ability to increase glucose-6-phosphate dehydrogenase activity and by maintaining optimal cellular detoxifying machinery. Exp Cell Res 247:61-78.
Quackenbush, J. (2006). Microarray analysis and tumor classification. N Engl J Med 354, 2463-2472.
Rahman, Q., Abidi, P., Afaq, F., Schiffmann, D., Mossman, B. T., Kamp, D. W., and Athar, M. 1999. Glutathione redox system in oxidative lung injury. Crit. Rev Toxicol 29:543-568.
Rajasekaran N S, Connell P, Christians E S, Yan L J, Taylor R P, Orosz A, Zhang X Q, Stevenson T J, Peshock R M, Leopold J A, Barry W H, Loscalzo J, Odelberg S J, and Benjamin I J. Human alpha B-crystallin mutation causes oxido-reductive stress and protein aggregation cardiomyopathy in mice. Cell 130: 427-439, 2007
Rand J D, and Grant C M. The thioredoxin system protects ribosomes against stress-induced aggregation. Molecular biology of the cell 17: 387-401, 2006.
Rebrin I, Kamzalov S, and Sohal R S. Effects of age and caloric restriction on glutathione redox state in mice. Free radical biology & medicine 35: 626-635, 2003.
Rong, Y. S., Titen, S. W., Xie, H. B., Golic, M. M., Bastiani, M., Bandyopadhyay, P., Olivera, B. M., Brodsky, M., Rubin, G. M. and Golic, K. G. 2002 Targeted mutagenesis by homologous recombination in D. melanogaster. Genes Dev. 16, 1568-1581.
Russell, R. L., Siedlak, S. L., Raina, A. K., Bautista, J. M., Smith, M. A., and Perry, G. (1999). Increased neuronal glucose-6-phosphate dehydrogenase and sulfhydryl levels indicate reductive compensation to oxidative stress in Alzheimer disease. Arch. Biochem. Biophys. 370, 236-239.
Ruwende, C., Khoo, S. C., Snow, R. W., Yates, S. N., Kwiatkowski, D., Gupta, S., Warn, P., Allsopp, C. E., Gilbert, S. C., Peschu, N., et al. 1995. Natural selection of hemi- and heterozygotes for G6PD deficiency in Africa by resistance to severe malaria. Nature 376:246-249.
Sam, F., Kerstetter, D. L., Pimental, D. R., Mulukutla, S., Tabaee, A., Bristow, M. R., Colucci, W. S., and Sawyer, D. B. (2005). Increased reactive oxygen species production and functional alterations in antioxidant enzymes in human failing myocardium. J. Card. Fail. 11, 473-480.
Sanbe A, Osinska H, Saffitz J E, Glabe C G, Kayed R, Maloyan A, and Robbins J. Desmin-related cardiomyopathy in transgenic mice: a cardiac amyloidosis. Proceedings of the National Academy of Sciences of the United States of America 101: 10132-10136, 2004.
Sano, M., Fukuda, K., Sato, T., Kawaguchi, H., Suematsu, M., Matsuda, S., Koyasu, S., Matsui, H., Yamauchi-Takihara, K., Harada, M., et al. 2001. ERK and p38 MAPK, but not NF-kappaB, are critically involved in reactive oxygen species-mediated induction of IL-6 by angiotensin II in cardiac fibroblasts. Circ Res 89:661-669.
Seidman, J. G., and Seidman, C. (2001). The genetic basis for cardiomyopathy: from mutation identification to mechanistic paradigms. Cell 104, 557-567.
Selcen, D., Ohno, K., and Engel, A. G. (2004). Myofibrillar myopathy: clinical, morphological and genetic studies in 63 patients. Brain 127, 439-451.
Seo, D., Ginsburg, G. S. & Goldschmidt-Clermont, P. J. (2006) J Am Coll Cardiol 48, 227-35.
Shacter, E., Williams, J. A., Lim, M., and Levine, R. L. 1994. Differential susceptibility of plasma proteins to oxidative modification: examination by western blot immunoassay. Free Radic Biol Med 17:429-437.
Simons, J. F., Ferro-Novick, S., Rose, M. D., and Helenius, A. (1995). BiP/Kar2p serves as a molecular chaperone during carboxypeptidase Y folding in yeast. J. Cell Biol. 130, 41-49.
Simonsen, A., Birkeland, H. C., Gillooly, D. J., Mizushima, N., Kuma, A., Yoshimori, T., Slagsvold, T., Brech, A. & Stenmark, H. (2004) J Cell Sci 117, 4239-51.
Stadtman, E. R. 1992. Protein oxidation and aging. Science 257:1220-1224.
Stefani M, and Dobson C M. Protein aggregation and aggregate toxicity: new insights into protein folding, misfolding diseases and biological evolution. Journal of molecular medicine (Berlin, Germany) 81: 678-699, 2003.
Sutherland, F. J., Baker, K. E., Shattock, M. J., and Hearse, D. J. (2003). Responses to ischaemia and reperfusion in the mouse isolated perfused heart and the phenomenon of ‘contractile cycling’. Clinical and Experimental Pharmacology & Physiology 30, 879-884.
Sutherland, F. J., Shattock, M. J., Baker, K. E., and Hearse, D. J. 2003. Mouse isolated perfused heart: characteristics and cautions. Clin Exp Pharmacol Physiol 30:867-878.
Takahashi M, Hakamata Y, Murakami T, et al. Establishment of lacZ-transgenic rats: a tool for regenerative research in myocardium. Biochem Biophys Res Commun. Jun. 13 2003; 305(4):904-908.
Tan, F. L., Moravec, C. S., Li, J., Apperson-Hansen, C., McCarthy, P. M., Young, J. B., and Bond, M. (2002). The gene expression fingerprint of human heart failure. Proc Natl Acad Sci USA 99, 11387-11392.
Taylor, R. P., and Benjamin, I. J. 2005. Small heat shock proteins: a new classification scheme in mammals. J Mol Cell Cardiol 38:433-444.
Taylor, S. W., Fahy, E., Zhang, B., Glenn, G. M., Warnock, D. E., Wiley, S., Murphy, A. N., Gaucher, S. P., Capaldi, R. A., Gibson, B. W. & Ghosh, S. S. (2003) Nat Biotechnol 21, 281-6.
Teichholz, L. E., Kreulen, T., Herman, M. V. & Gorlin, R. (1976) Am J Cardiol 37, 7-11.
Tome, M. E., Johnson, D. B., Samulitis, B. K., Dorr, R. T., and Briehl, M. M. 2006. Glucose 6-phosphate dehydrogenase overexpression models glucose deprivation and sensitizes lymphoma cells to apoptosis. Antioxid Redox Signal 8:1315-1327.
Trotter, E. W., and Grant, C. M. (2002). Thioredoxins are required for protection against a reductive stress in the yeast Saccharomyces cerevisiae. Mol Microbiol 46, 869-878.
Ursini, F., Maiorino, M., Brigelius-Flohe, R., Aumann, K. D., Roveri, A., Schomburg, D., and Flohe, L. (1995). Diversity of glutathione peroxidases. Methods in Enzymology 252, 38-53.
VanBuren V, Piao Y, Dudekula D B, Qian Y, Carter M G, Martin P R, Stagg C A, Bassey U C, Aiba K, Hamatani T, Kargul G J, Luo A G, Kelso J, Hide W, and Ko M S. Assembly, verification, and initial annotation of the NIA mouse 7.4K cDNA clone set. Genome research 12: 1999-2003, 2002
Vasan, R. S. (2006). Biomarkers of cardiovascular disease: molecular basis and practical considerations. Circulation 113, 2335-2362.
Vicart, P., Caron, A., Guicheney, P., Li, Z., Prevost, M. C., Faure, A., Chateau, D., Chapon, F., Tome, F., Dupret, J. M., et al. 1998. A missense mutation in the alphaB-crystallin chaperone gene causes a desmin-related myopathy. Nat Genet 20:92-95.
Wang, X., Osinska, H., Dorn, G. W., 2nd, Nieman, M., Lorenz, J. N., Gerdes, A. M., Witt, S., Kimball, T., Gulick, J., and Robbins, J. 2001. Mouse model of desmin-related cardiomyopathy. Circulation 103:2402-2407.
Wang, X., Osinska, H., Klevitsky, R., Gerdes, A. M., Nieman, M., Lorenz, J., Hewett, T., and Robbins, J. 2001. Expression of R120G-alphaB-crystallin causes aberrant desmin and alphaB-crystallin aggregation and cardiomyopathy in mice. Circ Res 89:84-91.
Wernicke D, Thiel C, Duja-Isac C M, et al. alpha-Tropomyosin mutations Asp(175)Asn and Glu(180)Gly affect cardiac function in transgenic rats in different ways. Am J Physiol Regul Integr Comp Physiol. September 2004; 287(3):R685-695.
Wiesenfeld, S. L., Petrakis, N. L., Sams, B. J., Collen, M. F., and Cutler, J. L. 1970. Elevated blood pressure, pulse rate and serum creatinine in Negro males deficient in glucose-6-phosphate dehydrogenase. N Engl J Med 282:1001-1002.
Williams, R. S., and Benjamin, I. J. 2000. Protective responses in the ischemic myocardium. J Clin Invest 106:813-818.
Xiao X, and Benjamin I J. Stress-response proteins in cardiovascular disease. American journal of human genetics 64: 685-690, 1999.
Yamamoto, M., Yang, G., Hong, C., Liu, J., Holle, E., Yu, X., Wagner, T., Vatner, S. F., and Sadoshima, J. 2003 Inhibition of endogenous thioredoxin in the heart increases oxidative stress and cardiac hypertrophy. J Clin Invest 112:1395-1406.
Yan, L. J., and Sohal, R. S. 2000. Analysis of oxidative modification of proteins. In Current protocols in protein sciences. J. E. Coligan, B. M. Dunn, H. L. Ploegh, D. W. Speicher, and P. T. Wingfield, editors. New York: John Wiley & Sons, Inc. 14.14.11-14.14.24.
Yan, L. J., Christians, E. S., Liu, L., Xiao, X., Sohal, R. S., and Benjamin, I. J. 2002. Mouse heat shock transcription factor 1 deficiency alters cardiac redox homeostasis and increases mitochondrial oxidative damage. Embo J 21:5164-5172.
Yan, L. J., Rajasekaran, N. S., Sathyanarayanan, S., and Benjamin, I. J. 2005. Mouse HSF1 disruption perturbs redox state and increases mitochondrial oxidative stress in kidney. Antioxid Redox Signal 7:465-471.
Yang Y, Song Y, and Loscalzo J. Regulation of the protein disulfide proteome by mitochondria in mammalian cells. Proceedings of the National Academy of Sciences of the United States of America 104: 10813-10817, 2007
Ye, G., Metreveli, N. S., Donthi, R. V., Xia, S., Xu, M., Carlson, E. C., and Epstein, P. N. 2004. Catalase protects cardiomyocyte function in models of type 1 and type 2 diabetes. Diabetes 53:1336-1343.

H. SEQUENCES

1. SEQ ID NO: 1 - Human CryAB (ACCESSION NP_001876)

| | |
|---|---|
| 1 | mdiaihhpwi rrpffpfhsp srlfdqffge hllesdlfpt stslspfylr ppsflrapsw |
| 61 | fdtglsemrl ekdrfsvnld vkhfspeelk vkvlgdviev hgkheerqde hgfisrefhR |
| 121 | kyripadvdp ltitsslssd gvltvngprk qvsgpertrp itreekpavt aapkk |

2. SEQ ID NO: 2 - Human CryAB (ACCESSION NM_001885)

| | |
|---|---|
| 1 | gacccctcac actcacctag ccaccatgga catcgccatc caccacccct ggatccgccg |
| 61 | ccccttcttt cctttccact cccccagccg cctctttgac cagttcttcg gagagcacct |
| 121 | gttggagtct gatcttttcc cgacgtctac ttccctgagt cccttctacc ttcggccacc |
| 181 | ctccttcctg cgggcaccca gctggtttga cactggactc tcagagatgc gcctggagaa |
| 241 | ggacaggttc tctgtcaacc tggatgtgaa gcacttctcc ccagaggaac tcaaagttaa |
| 301 | ggtgttggga gatgtgattg aggtgcatgg aaaacatgaa gagcgccagg atgaacatgg |
| 361 | tttcatctcc agggagttcc acAGGaaata ccggatccca gctgatgtag accctctcac |
| 421 | cattacttca tccctgtcat ctgatggggt cctcactgtg aatggaccaa ggaaacaggt |
| 481 | ctctggccct gagcgcacca ttcccatcac ccgtgaagag aagcctgctg tcaccgcagc |
| 541 | ccccaagaaa tagatgccct ttcttgaatt gcatttttta aaacaagaaa gtttccccac |
| 601 | cagtgaatga aagtcttgtg actagtgctg aagcttatta atgctaaggg caggcccaaa |
| 661 | ttatcaagct aataaaatat cattcagcaa c |

3. SEQ ID NO: 3 - Human R120GCryAB

| | |
|---|---|
| 1 | mdiaihhpwi rrpffpfhsp srlfdqffge hllesdlfpt stslspfylr ppsflrapsw |
| 61 | fdtglsemrl ekdrfsvnld vkhfspeelk vkvlgdviev hgkheerqde hgfisrefhG |
| 121 | kyripadvdp ltitsslssd gvltvngprk qvsgpertrp itreekpavt aapkk |

4. SEQ ID NO: 4 - Human R120GCryAB

| | |
|---|---|
| 1 | gacccctcac actcacctag ccaccatgga catcgccatc caccacccct ggatccgccg |
| 61 | ccccttcttt cctttccact cccccagccg cctctttgac cagttcttcg gagagcacct |
| 121 | gttggagtct gatcttttcc cgacgtctac ttccctgagt cccttctacc ttcggccacc |
| 181 | ctccttcctg cgggcaccca gctggtttga cactggactc tcagagatgc gcctggagaa |
| 241 | ggacaggttc tctgtcaacc tggatgtgaa gcacttctcc ccagaggaac tcaaagttaa |
| 301 | ggtgttggga gatgtgattg aggtgcatgg aaaacatgaa gagcgccagg atgaacatgg |
| 361 | tttcatctcc agggagttcc acGGAaaata ccggatccca gctgatgtag accctctcac |
| 421 | cattacttca tccctgtcat ctgatggggt cctcactgtg aatggaccaa ggaaacaggt |
| 481 | ctctggccct gagcgcacca ttcccatcac ccgtgaagag aagcctgctg tcaccgcagc |
| 541 | ccccaagaaa tagatgccct ttcttgaatt gcatttttta aaacaagaaa gtttccccac |
| 601 | cagtgaatga aagtcttgtg actagtgctg aagcttatta atgctaaggg caggcccaaa |
| 661 | ttatcaagct aataaaatat cattcagcaa c |

5. SEQ ID NO: 5 - Human R120GCryAB

| | |
|---|---|
| 1 | gacccctcac actcacctag ccaccatgga catcgccatc caccacccct ggatccgccg |
| 61 | ccccttcttt cctttccact cccccagccg cctctttgac cagttcttcg gagagcacct |
| 121 | gttggagtct gatcttttcc cgacgtctac ttccctgagt cccttctacc ttcggccacc |
| 181 | ctccttcctg cgggcaccca gctggtttga cactggactc tcagagatgc gcctggagaa |
| 241 | ggacaggttc tctgtcaacc tggatgtgaa gcacttctcc ccagaggaac tcaaagttaa |
| 301 | ggtgttggga gatgtgattg aggtgcatgg aaaacatgaa gagcgccagg atgaacatgg |
| 361 | tttcatctcc agggagttcc acGGCaaata ccggatccca gctgatgtag accctctcac |
| 421 | cattacttca tccctgtcat ctgatggggt cctcactgtg aatggaccaa ggaaacaggt |
| 481 | ctctggccct gagcgcacca ttcccatcac ccgtgaagag aagcctgctg tcaccgcagc |
| 541 | ccccaagaaa tagatgccct ttcttgaatt gcatttttta aaacaagaaa gtttccccac |
| 601 | cagtgaatga aagtcttgtg actagtgctg aagcttatta atgctaaggg caggcccaaa |
| 661 | ttatcaagct aataaaatat cattcagcaa c |

6. SEQ ID NO: 6 - Human R120GCryAB

| | |
|---|---|
| 1 | gacccctcac actcacctag ccaccatgga catcgccatc caccacccct ggatccgccg |
| 61 | ccccttcttt cctttccact cccccagccg cctctttgac cagttcttcg gagagcacct |
| 121 | gttggagtct gatcttttcc cgacgtctac ttccctgagt cccttctacc ttcggccacc |
| 181 | ctccttcctg cgggcaccca gctggtttga cactggactc tcagagatgc gcctggagaa |
| 241 | ggacaggttc tctgtcaacc tggatgtgaa gcacttctcc ccagaggaac tcaaagttaa |
| 301 | ggtgttggga gatgtgattg aggtgcatgg aaaacatgaa gagcgccagg atgaacatgg |
| 361 | tttcatctcc agggagttcc acGGGaaata ccggatccca gctgatgtag accctctcac |
| 421 | cattacttca tccctgtcat ctgatggggt cctcactgtg aatggaccaa ggaaacaggt |
| 481 | ctctggccct gagcgcacca ttcccatcac ccgtgaagag aagcctgctg tcaccgcagc |
| 541 | ccccaagaaa tagatgccct ttcttgaatt gcatttttta aaacaagaaa gtttccccac |
| 601 | cagtgaatga aagtcttgtg actagtgctg aagcttatta atgctaaggg caggcccaaa |
| 661 | ttatcaagct aataaaatat cattcagcaa c |

7. SEQ ID NO: 7 - Human R120GCryAB

| | |
|---|---|
| 1 | gacccctcac actcacctag ccaccatgga catcgccatc caccacccct ggatccgccg |
| 61 | ccccttcttt cctttccact cccccagccg cctctttgac cagttcttcg gagagcacct |
| 121 | gttggagtct gatcttttcc cgacgtctac ttccctgagt cccttctacc ttcggccacc |
| 181 | ctccttcctg cgggcaccca gctggtttga cactggactc tcagagatgc gcctggagaa |
| 241 | ggacaggttc tctgtcaacc tggatgtgaa gcacttctcc ccagaggaac tcaaagttaa |
| 301 | ggtgttggga gatgtgattg aggtgcatgg aaaacatgaa gagcgccagg atgaacatgg |
| 361 | tttcatctcc agggagttcc acGGTaaata ccggatccca gctgatgtag accctctcac |
| 421 | cattacttca tccctgtcat ctgatggggt cctcactgtg aatggaccaa ggaaacaggt |
| 481 | ctctggccct gagcgcacca ttcccatcac ccgtgaagag aagcctgctg tcaccgcagc |
| 541 | ccccaagaaa tagatgccct ttcttgaatt gcatttttta aaacaagaaa gtttccccac |
| 601 | cagtgaatga aagtcttgtg actagtgctg aagcttatta atgctaaggg caggcccaaa |
| 661 | ttatcaagct aataaaatat cattcagcaa c |

8. SEQ ID NO: 8
AACCTGCTAGACCACCTGGA
9. SEQ ID NO: 9
GGAACCTGTTGCAGCCTAGT
10. SEQ ID NO: 10
CACTGAAGTTGTTGTAGGAAGACC
11. SEQ ID NO: 11
CAAAACCAGGAAATACGCTATG
12. SEQ ID NO: 12
TCATCTCCAGGGAGTTCCAC
13. SEQ ID NO: 13
TAATCTGGGCCAGCCCTTAG
14. SEQ ID NO: 14
GCTGCCAATTTCCTCAACAT
15. SEQ ID NO: 15
ATCACAGCCAACACAGCAAG

What is claimed is:

1.-43. (canceled)

44. A method of treating heart failure in a subject, comprising administering to the subject a composition comprising an anti-reductant molecule.

45. The method of claim 44, wherein the anti-reluctant molecule comprises a thiuram disulfide compound selected from the group consisting of diethyldithiocarbamate, pyrrolidinedithiocarbamate, N-methyl, N-ethyldithiocarbamates, hexamethylenedithiocarbamate, imadazolinedithiocarbamates, dibenzyldithiocarbamate, dimethylenedithiocarbamate, dipopyldithiocarbamate, dibutyldithiocarbamate, diamyldithiocarbamate, N-methyl, N-cyclopropylmethyldithiocarbamate, cyclohexylamyldithiocarbamate pentamethylenedithiocarbamate, dihydroxyethyldithiocarbamate, N-methylglucosamine dithiocarbamate, and salts and derivatives thereof.

46. The method of claim 44, wherein the anti-reluctant molecule comprises tetraethylthiuram disulfide (disulfuram).

47. The method of claim 44, wherein the method further comprises administering to the subject a heavy metal ion.

48. The method of claim 47, wherein the heavy metal ion is selected from the group consisting of arsenic, bismuth, cobalt, copper, chromium, gallium, gold, iron, manganese, nickel, silver, titanium, vanadium, selenium, and zinc.

49. The method of claim 47, wherein the thiuram disulfide and heavy metal ion are administered separately.

50. The method of claim 47, wherein the thiuram disulfide and heavy metal ion are administered in combination.

51. The method of claim 50, wherein the thiuram disulfide and heavy metal ion are administered as a chelating complex.

52. The method of claim 44, wherein the anti-reductant molecule comprises an inhibitor of glucose-6-phosphate dehydrogenase (G6PD).

53. The method of claim 52, wherein the inhibitor of G6PD comprises dehydroepiandrosterone (DHEA), DHEA-sulfate (DHEA-S), 16α-bromoepiandrosterone (EPI), or 16 alpha-fluoro-5-androsten-17-one (fluasterone).

54. The method of claim 44, wherein the subject comprises a mutation in αB-crystallin (CryAB) or desmin.

55. The method of claim 44, wherein the subject comprises a R120G mutation in CryAB (R120GCryAB).

56. The method of claim 44, wherein the method further comprises diagnosing the subject prior to administration of the anti-reductant molecule.

57. A method of treating muscular dystrophy in a subject, comprising administering to the subject a composition comprising a thiuram disulfide compound.

58. The method of claim 57, wherein the thiuram disulfide compound is selected from the group consisting of diethyldithiocarbamate, pyrrolidinedithiocarbamate, N-methyl, N-ethyldithiocarbamates, hexamethylenedithiocarbamate, imadazolinedithiocarbamates, dibenzyldithiocarbamate, dimethylenedithiocarbamate, dipopyldithiocarbamate, dibutyldithiocarbamate, diamyldithiocarbamate, N-methyl, N-cyclopropylmethyldithiocarbamate, cyclohexylamyldithiocarbamate pentamethylenedithiocarbamate, dihydroxyethyldithiocarbamate, N-methylglucosamine dithiocarbamate, and salts and derivatives thereof.

59. The method of claim 57, wherein the thiuram disulfide is tetraethylthiuram disulfide (disulfuram).

60. The method of claim 57, wherein the method further comprises administering to the subject a heavy metal ion.

61. The method of claim 60, wherein the heavy metal ion is selected from the group consisting of arsenic, bismuth, cobalt, copper, chromium, gallium, gold, iron, manganese, nickel, silver, titanium, vanadium, selenium, and zinc.

62. The method of claim 60, wherein the thiuram disulfide and heavy metal ion are administered separately.

63. The method of claim 60, wherein the thiuram disulfide and heavy metal ion are administered in combination.

64. The method of claim 63, wherein the thiuram disulfide and heavy metal ion are administered as a chelating complex.


SEQUENCE LISTING (15 sequences)

SEQ ID NO: 1 - protein, 175 aa, Artificial Sequence
  Description of Artificial Sequence  note = Synthetic Construct
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 2 - DNA, 691 bp, Artificial Sequence
  Description of Artificial Sequence  note = Synthetic Construct
gacccctcacactcacctagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatagatgccctttcttgaattgcattttttaaaacaagaaagtttccccaccagtgaatgaaagtcttgtgactagtgctgaagcttattaatgctaagggcaggcccaaattatcaagctaataaaatatcattcagcaac

SEQ ID NO: 3 - protein, 175 aa, Artificial Sequence
  Description of Artificial Sequence  note = Synthetic Construct
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHGKYRIPADVDPLTITSSLSSDGVLTVNGPRKQVSGPERTIPITREEKPAVTAAPKK

SEQ ID NO: 4 - DNA, 691 bp, Artificial Sequence
  Description of Artificial Sequence  note = Synthetic Construct
gacccctcacactcacctagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacggaaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatagatgccctttcttgaattgcattttttaaaacaagaaagtttccccaccagtgaatgaaagtcttgtgactagtgctgaagcttattaatgctaagggcaggcccaaattatcaagctaataaaatatcattcagcaac

SEQ ID NO: 5 - DNA, 691 bp, Artificial Sequence
  Description of Artificial Sequence  note = Synthetic Construct
gacccctcacactcacctagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacggcaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatagatgccctttcttgaattgcattttttaaaacaagaaagtttccccaccagtgaatgaaagtcttgtgactagtgctgaagcttattaatgctaagggcaggcccaaattatcaagctaataaaatatcattcagcaac

SEQ ID NO: 6 - DNA, 691 bp, Artificial Sequence
  Description of Artificial Sequence  note = Synthetic Construct
gacccctcacactcacctagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacgggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatagatgccctttcttgaattgcattttttaaaacaagaaagtttccccaccagtgaatgaaagtcttgtgactagtgctgaagcttattaatgctaagggcaggcccaaattatcaagctaataaaatatcattcagcaac

SEQ ID NO: 7 - DNA, 691 bp, Artificial Sequence
  Description of Artificial Sequence  note = Synthetic Construct
gacccctcacactcacctagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacggtaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatagatgccctttcttgaattgcattttttaaaacaagaaagtttccccaccagtgaatgaaagtcttgtgactagtgctgaagcttattaatgctaagggcaggcccaaattatcaagctaataaaatatcattcagcaac

SEQ ID NO: 8 - DNA, 20 bp, Artificial Sequence
  Description of Artificial Sequence  note = Synthetic Construct
aacctgctagaccacctgga

SEQ ID NO: 9 - DNA, 20 bp, Artificial Sequence
  Description of Artificial Sequence  note = Synthetic Construct
ggaagctgttgcagcctagt

SEQ ID NO: 10 - DNA, 24 bp, Artificial Sequence
  Description of Artificial Sequence  note = Synthetic Construct
cactgaagttgttgtaggaagacc

SEQ ID NO: 11 - DNA, 22 bp, Artificial Sequence
  Description of Artificial Sequence  note = Synthetic Construct
caaaagcaggaaatacgctatg

SEQ ID NO: 12 - DNA, 20 bp, Artificial Sequence
  Description of Artificial Sequence  note = Synthetic Construct
tcatctccagggagttccac

SEQ ID NO: 13 - DNA, 20 bp, Artificial Sequence
  Description of Artificial Sequence  note = Synthetic Construct
taatctgggccagcccttag

SEQ ID NO: 14 - DNA, 20 bp, Artificial Sequence
  Description of Artificial Sequence  note = Synthetic Construct
gctgccaatttcctcaacat

SEQ ID NO: 15 - DNA, 20 bp, Artificial Sequence
  Description of Artificial Sequence  note = Synthetic Construct
atcacagccaacacagcaag